SEQUENCE LISTING (21 sequences)

SEQ ID NO: 1 - DNA, 121 bp, Homo sapiens
  misc_feature (61)..(61): n=a or g
ttcatgcacccagcagactaaatgtttactgagtacttaccgaaggttaggatctgggctnagggttgaaagaaataaataggttaaaaaagaaaaaaagccacctaggtgactttcactc

SEQ ID NO: 2 - DNA, 52 bp, Homo sapiens
  misc_feature (27)..(27): n=a or g
cattgattttaaacctcaacaacagcnatgtcttttatcagcttaattttac

SEQ ID NO: 3 - DNA, 121 bp, Homo sapiens
  misc_feature (61)..(61): n=a or g
cctgtgtggagcccattacctgaagaggggccaagaggacaagcaggtatgactatggtcnggcgtgccaagtcccaggacaaggaaggacgggtgctccaggaagcacaggagggggcat

SEQ ID NO: 4 - DNA, 121 bp, Homo sapiens
  misc_feature (61)..(61): n=t or c
taccggatggcagtgagcagggaggctcacctggatcatttggtgaaggtggcatctgccnggtttgtccactgtgaagttcctattcctaccccgccccccacctttcttttttgagatg

SEQ ID NO: 5 - DNA, 121 bp, Homo sapiens
  misc_feature (61)..(61): n=t or c
acttaactatttgttgggtgaatatagaaatgaatgaatgaatggatggatgagcagatanatcaagaagttaattcacaaattaaagcccattatgaaactaaagtagaggctgggcgcg

SEQ ID NO: 6 - DNA, 121 bp, Homo sapiens
  misc_feature (61)..(61): n=a or g
acccgtgaacaagtcgggcccccatccacgcaatatctgcagtctcgactgtatgatctcntcctttgcagccacactgtgaggcagcaatgatcattccgcagacggccacagactccag

SEQ ID NO: 7 - DNA, 121 bp, Homo sapiens
  misc_feature (61)..(61): n=t or c
gacgacacccagcacacccagcacacccagcacaccagcgaacagcccaccaggtgctatngctgtcattcatttgctcattcgctcgttcatgcacccagcagactaaatgtttactgag

SEQ ID NO: 8 - DNA, 121 bp, Homo sapiens
  misc_feature (61)..(61): n=t or c
aaaacagtgctccaaaggcaaagaaatagcaaagacagaagtaaggcacttaactatttgntgggtgaatatagaaatgaatgaatgaatggatggatgagcagatacatcaagaagttaa

SEQ ID NO: 9 - DNA, 121 bp, Homo sapiens
  misc_feature (61)..(61): n=t or c
ggcagctatgtaggaagcagtgaagatccacatccttccttattggtgaaaggaatgaatnggaaacagaaagttcttttttacctttattaaataaacgtgaagtcataagaactactaa

SEQ ID NO: 10 - DNA, 121 bp, Homo sapiens
  misc_feature (61)..(61): n=t or c
agactttgtctcaaaaaagaaaaaaaaaaaaaaagaagtcccaaataataaaatatgaganggatttatggaagaaagtgaaagaaacaaagggtaggcaccttgcctgtttaatttgatc

SEQ ID NO: 11 - DNA, 121 bp, Homo sapiens
  misc_feature (61)..(61): n=a or g
tggatggatgagcagatacatcaagaagttaattcacaaattaaagcccattatgaaactnaagtagaggctgggcgcggtggatcacgcctataatcccagcactttgggaggtcaaggc

SEQ ID NO: 12 - DNA, 121 bp, Homo sapiens
  misc_feature (61)..(61): n=t or g
tgtgatatgatggtcatatcatagcacagggctgttgtgaggattaaatgagttgattcangtaaacagggacatccgaaaaagggaaagacggtgcttgtcctgagaacagctgtgaatg

SEQ ID NO: 13 - DNA, 121 bp, Homo sapiens
  misc_feature (61)..(61): n=a or g
aggtgagtggccttaaaggggaaggagaaaccttttgaaagcaggacaggtcctctctgantcatccccgtatgggtaaatctacatcactagcttcattactgactggtccatgtagaaa

SEQ ID NO: 14 - DNA, 121 bp, Homo sapiens
  misc_feature (61)..(61): n=a or g
caggtatgtcttcaaacctatgatggataaaagttacagtcagcacagattgaaagcaccntctgttgaaacgcagctccgtcttgctctctggagaggactcactcctggaaagttgaga

SEQ ID NO: 15 - DNA, 121 bp, Homo sapiens
  misc_feature (61)..(61): n=a or g
tgtaaccaagtaaccaatggtaaacctctacagggtattaaggctccagaaaattctctantcagccacttgctcctgctcgagcctgctcccactccgtggagtgtactttcatttcagt

SEQ ID NO: 16 - DNA, 52 bp, Homo sapiens
  misc_feature (27)..(27): n=c, g or t
tttggggtgacgaaaatgtaaaattangttgtggtgatggttgcacaacacc

SEQ ID NO: 17 - DNA, 52 bp, Homo sapiens
  misc_feature (27)..(27): n=g or t
gaataaccacacacatggaccctgggntccaagttcattagaatggctcttt

SEQ ID NO: 18 - DNA, 52 bp, Homo sapiens
  misc_feature (27)..(27): n=g or t
aagacagaggaacccccataggctggnggtgagcagggggcatgagggctaa

SEQ ID NO: 19 - DNA, 52 bp, Homo sapiens
  misc_feature (27)..(27): n=c or t
tgtccaactatttctttctttcttttntgagatgggggtctcactgtgttgg

SEQ ID NO: 20 - DNA, 52 bp, Homo sapiens
  misc_feature (27)..(27): n=c or t
ttaacctatttatttctttcaaccctnagcccagatcctaaccttcggtaag

SEQ ID NO: 21 - DNA, 51 bp, Homo sapiens
  misc_feature (26)..(26): n=c or g
ggacctgcctggtgctttctcagagnagactgaggtttggggtttgcggaa

(19) United States
(12) Patent Application Publication
(10) Pub. No.: US 2021/0236442 A1
(43) Pub. Date: Aug. 5, 2021

(54) METHODS FOR DELAYING OCCURRENCE OF NEW-ONSET TYPE 2 DIABETES AND FOR SLOWING PROGRESSION OF AND TREATING TYPE 2 DIABETES

(71) Applicant: Dalcor Pharma UK Ltd., Leatherhead, Zug Branch, Zug (CH)

(72) Inventors: Marie-Pierre DUBÉ, Montreal (CA); Jean-Claude TARDIF, Montreal (CA); Fouzia LAGHRISSI-THODE, Hurden (CH)

(21) Appl. No.: 17/169,301

(22) Filed: Feb. 5, 2021

Related U.S. Application Data
(63) Continuation of application No. PCT/EP2019/071506, filed on Aug. 9, 2019.
(60) Provisional application No. 62/716,630, filed on Aug. 9, 2018.
(60) Provisional application No. 62/716,639, filed on Aug. 9, 2018.

(51) Int. Cl. A61K 31/167; A61P 3/10; A61K 45/06; A61K 38/28; A61K 31/155; A61K 31/64; A61K 38/22

(52) U.S. Cl. CPC A61K 31/167; A61P 3/10; A61K 45/06; A61K 38/22; A61K 31/155; A61K 31/64; A61K 38/28

(57) ABSTRACT

The invention provides compositions and methods useful for delaying occurrence of new-onset type 2 diabetes, slowing progression of type 2 diabetes, treating type 2 diabetes, and slowing progression of a complication of type 2 diabetes.

### CROSS-REFERENCE TO RELATED APPLICATIONS

The present application is a continuation of PCT Application No. PCT/EP2019/071506, filed Aug. 9, 2019, which claims the benefit of U.S. Provisional Patent Application Nos. 62/716,630, filed Aug. 9, 2018, and 62/716,639, filed Aug. 9, 2018, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is DLCR_004_01WO_SeqList_ST25. The text file is about 7 kilobytes, was created on Jul. 31, 2019 and is being submitted electronically via EFS-Web.

### FIELD OF THE INVENTION

The present disclosure provides methods useful for delaying occurrence of new-onset type 2 diabetes, slowing progression of type 2 diabetes, treating type 2 diabetes, and slowing progression of a complication of type 2 diabetes.

### BACKGROUND

Diabetes is a group of diseases characterized by high blood glucose levels, which result from defects in insulin production, insulin action, or both. Diabetes is a chronic disease that presently has no cure. There are two generally recognized forms of diabetes, type 1 and type 2. Type 1 diabetes develops when the body's immune system destroys pancreatic cells that make the hormone insulin, which regulates blood glucose levels. Type 1 diabetes usually occurs in children and young adults; although disease onset can occur at any age. Type 1 diabetes is typically treated with exogenous insulin administered via injection. Type 2 diabetes is a metabolic disorder resulting from the body's inability to make enough, or properly use, insulin. This disease usually begins as insulin resistance, a disorder in which the cells do not use insulin properly, and as the need for insulin rises, the pancreas gradually loses its ability to produce insulin. Type 2 diabetes is the most common form of the disease accounting for 90-95 percent of diabetes.

While diabetes is often linked with high LDL cholesterol and low HDL cholesterol, the ability of a cholesteryl ester transfer protein (“CETP”) inhibitor to exert glycemic control, especially in patients with varied genetics, has not yet been demonstrated. Diabetic patients are recognized to be at high risk for cardiovascular events, therefore new treatments for Type 2 diabetes should provide cardiovascular safety.

### SUMMARY OF THE INVENTION

One aspect of the invention provides methods for delaying occurrence of new-onset type 2 diabetes, comprising administering an effective amount of a CETP inhibitor to a subject in need thereof and known to have genotype rs1967309/AA or rs1967309/AG.

Another aspect of the invention provides methods for slowing progression of type 2 diabetes, comprising administering an effective amount of a CETP inhibitor to a subject in need thereof and known to have genotype rs1967309/AA or rs1967309/AG.

Another aspect of the invention provides methods for treating type 2 diabetes, comprising administering an effective amount of a CETP inhibitor to a subject in need thereof and known to have genotype rs1967309/AA or rs1967309/AG.

Another aspect of the invention provides methods for slowing progression of a complication of type 2 diabetes, comprising administering an effective amount of a CETP inhibitor to a subject in need thereof and known to have genotype rs1967309/AA or rs1967309/AG.

Another aspect of the invention provides methods for delaying occurrence of new-onset type 2 diabetes, comprising administering to a subject in need thereof an effective amount of: (a) a CETP inhibitor; and (b) an ADCY inhibitor.

Another aspect of the invention provides methods for slowing progression of type 2 diabetes, comprising administering to a subject in need thereof an effective amount of: (a) a CETP inhibitor; and (b) an ADCY inhibitor.

Another aspect of the invention provides methods for treating type 2 diabetes, comprising administering to a subject in need thereof an effective amount of: (a) a CETP inhibitor; and (b) an ADCY inhibitor.

Another aspect of the invention provides methods for slowing progression of a complication of type 2 diabetes, comprising administering to a subject in need thereof an effective amount of: (a) a CETP inhibitor; and (b) an ADCY inhibitor.

Each of the aforementioned methods is a “method of the invention”.

Another aspect of the invention provides compositions comprising (a) an effective amount of a CETP inhibitor and an antidiabetic agent; and (b) a pharmaceutically acceptable carrier or vehicle.

Another aspect of the invention provides compositions comprising (a) an effective amount of a CETP inhibitor, an ADCY inhibitor and an antidiabetic agent; and (b) a pharmaceutically acceptable carrier or vehicle.

Each of the aforementioned compositions is a “composition of the invention”.

### BRIEF DESCRIPTION OF THE FIGURES

FIG. 1 is a bar graph that shows placebo-adjusted geometric mean percentage change in hemoglobin A1c (“HbA1c”) in diabetic and non-diabetic patients at six months (“M06”) from baseline according to ADCY9 genotype.

FIG. 2 is a bar graph that shows placebo-adjusted geometric mean percentage change in HbA1c in diabetic and non-diabetic patients at twelve months (“M12”) from baseline according to ADCY9 genotype.

FIG. 3 is a bar graph that shows placebo-adjusted geometric mean percentage change in HbA1c in diabetic and non-diabetic patients at 24 months (“M24”) from baseline according to ADCY9 genotype.

FIG. 4 is a bar graph that shows placebo-adjusted geometric mean percentage change in HbA1c in uncontrolled diabetic patients at M06 from baseline according to ADCY9 genotype.

### DETAILED DESCRIPTION OF THE INVENTION

### Definitions

An “effective amount” as used herein in connection with a CETP inhibitor, refers to an amount of CETP inhibitor that is effective for delaying occurrence of new-onset type 2 diabetes, slowing progression of type 2 diabetes, treating type 2 diabetes or slowing progression of a complication of type 2 diabetes in a subject. An “effective amount” as used herein in connection with a CETP inhibitor and an ACDY inhibitor, refers to the total amount of CETP inhibitor and ADCY inhibitor that is effective for delaying occurrence of new-onset type 2 diabetes, slowing progression of type 2 diabetes, treating type 2 diabetes or slowing progression of a complication of type 2 diabetes in a subject.

“HbA1c” is a marker that is useful for monitoring blood glucose. See Diabetes Res Clin Pract. 2014 April; 104(1):1-52; and World Health Organization, Use of Glycated Haemoglobin (HbA1c) in the Diagnosis of Diabetes Mellitus: Abbreviated Report of a WHO Consultation. 2011. pp. 1-25.

The term “about” when used in connection with a referenced numeric indication means the referenced numeric indication plus or minus up to 10% of that referenced numeric indication. For example, the language “about 50” means from 45 to 55.

The term “subject,” as used herein unless otherwise defined, is a mammal, e.g., a human, mouse, rat, guinea pig, dog, cat, horse, cow, pig, or non-human primate, such as a monkey, chimpanzee, or baboon. In some embodiments, the subject is a human. In some embodiments, the subject is an adult human. In some embodiments, the subject is a pediatric human.

The language “known to have” as used herein in connection with a genotype means that a person performing the administering knows that the subject has the genotype. In some embodiments, the person is the subject. In some embodiments, the person is a healthcare provider.

As used herein, the term “adult human” refers to a human that is 18 years or older.

As used herein, the term “pediatric human” refers to a human that is 1 year to 18 years old.

### CEPT Inhibitors

CETP inhibitors that are useful in the compositions and methods of the invention include small molecules, anti-CETP antibodies and peptides that inhibit or suppress CETP activity.

CETP inhibitors that are useful in the compositions and methods of the invention include, but are not limited to, dalcetrapib, anacetrapib, evacetrapib, torcetrapib, BAY 60-5521, obicetrapib, BMS-795311, CP-800,569, DRL-17822, JNJ-28545595, JNJ-28614872, BAY 19-4789, BAY 38-1315, DLBS-1449 (Dexa Medica) and ATH-03 (Affris), and pharmaceutically acceptable salts of any of the foregoing.

“Dalcetrapib” refers to S-[2-({[1-(2-Ethylbutyl)cyclohexyl]carbonyl}amino)phenyl]-2-methylpropanethioate, and is also known as JTT-705 or CAS 211513-37-0. Dalcetrapib has the structure:

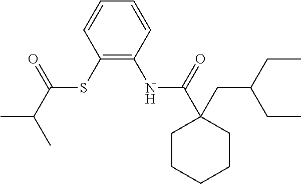 [SMILES: CCC(CC)CC1(C(=O)Nc2ccccc2SC(=O)C(C)C)CCCCC1]

“Anacetrapib” refers to (4S,5R)-5-[3,5-bis(trifluoromethyl)phenyl]-3-{[4′-fluoro-2′-methoxy-5′-(propan-2-yl)-4-(trifluoromethyl)[1,1′-biphenyl]-2-yl]methyl}-4-methyl-1,3-oxazolidin-2-one, and is also known as (4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxazolidin-2-one; MK-0859; or CAS 875446-37-0. Anacetrapib has the structure:

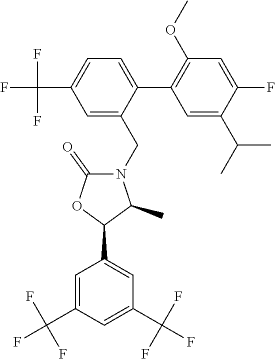 [SMILES: COc1cc(F)c(C(C)C)cc1-c1ccc(C(F)(F)F)cc1CN1C(=O)O[C@H](c2cc(C(F)(F)F)cc(C(F)(F)F)c2)[C@@H]1C]

“Evacetrapib” refers to trans-4-({(5S)-5-[{[3,5-bis(trifluoromethyl)phenyl]methyl}(2-methyl-2H-tetrazol-5-yl)amino]-7,9-dimethyl-2,3,4,5-tetrahydro-1H-benzazepin-1-yl}methyl)cyclohexanecarboxylic acid, and is also known as LY2484595 or CAS 1186486-62-3. Evacetrapib has the structure:

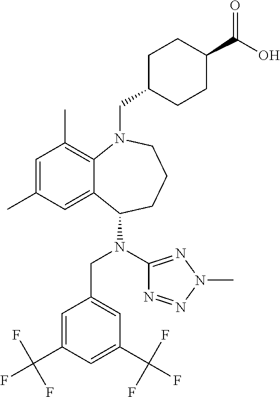 [SMILES: Cc1cc(C)c2c(c1)[C@@H](N(Cc1cc(C(F)(F)F)cc(C(F)(F)F)c1)c1nnn(C)n1)CCCN2C[C@H]1CC[C@H](C(=O)O)CC1]

“Torcetrapib” refers to (2R,4S)-4-[(3,5-bistrifluoromethylbenzyl) methoxycarbonylamino]-2-ethyl-6-trifluoromethyl-3,4-dihydro-2H-quinoline-1-carboxylic acid ethyl ester, and is also known as Ethyl (2R,4S)-4-({[3,5-bis(trifluoromethyl)phenyl]methyl}(methoxycarbonyl)amino)-2-ethyl-6-(trifluoromethyl)-1,2,3,4-tetrahydroquinoline-1-carboxylate; CP-529,414; or CAS 262352-17-0. Torcetrapib has the structure:

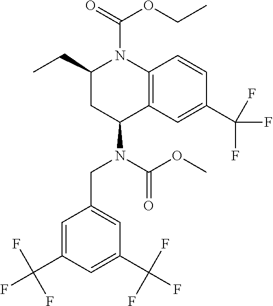 [SMILES: CCOC(=O)N1c2ccc(C(F)(F)F)cc2[C@@H](N(Cc2cc(C(F)(F)F)cc(C(F)(F)F)c2)C(=O)OC)C[C@H]1CC]

“BAY 60-5521” refers to (S)-4-cyclohexyl-2-cyclopentyl-3-((S)-fluoro(4-(trifluoromethyl)phenyl)methyl)-7,7-dimethyl-5,6,7,8-tetrahydroquinolin-5-ol, and is also known as CAS 893409-49-9. BAY 60-5521 has the structure.

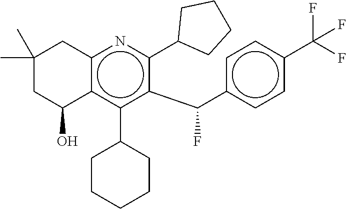

“Obicetrapib” refers to 4-((2-((3,5-bis(trifluoromethyl)benzyl)((2R,4S)-1-(ethoxycarbonyl)-2-ethyl-6-(trifluoromethyl)-1,2,3,4-tetrahydroquinolin-4-yl)amino)pyrimidin-5-yl)oxy)butanoic acid, and is also known as AMG-899, DEZ-001, TA-8995 or CAS 866399-87-3. Obicetrapib has the structure.

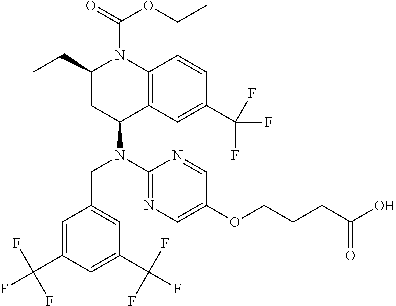 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

“BMS795311” refers to (R)—N-(1-(3-cyclopropoxy-4-fluorophenyl)-1-(3-fluoro-5-(2,2,3,3-tetrafluoropropanoyl)phenyl)-2-phenylethyl)-4-fluoro-3-(trifluoromethyl)benzamide, and is also known as CAS 939390-99-5. BMS795311 has the structure:

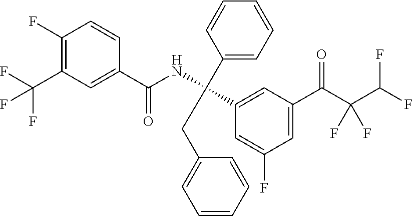 [SMILES: O=C(N[C@](Cc1ccccc1)(c1ccccc1)c1cc(F)cc(C(=O)C(F)(F)C(F)F)c1)c1ccc(F)c(C(F)(F)F)c1]

“CP-800,569” refers to (2R)-3-[3-(4-chloro-3-ethylphenoxy)-n-[[3-(1,1,2,2-tetrafluoroethoxy)phenyl]methyl]anilino]-1,1,1-trifluoropropan-2-ol. CP-800,569 has the structure:

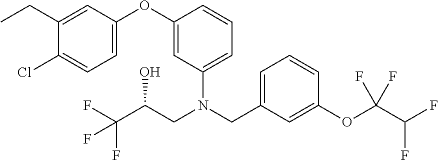 [SMILES: CCc1cc(Oc2cccc(N(Cc3cccc(OC(F)(F)C(F)F)c3)C[C@@H](O)C(F)(F)F)c2)ccc1Cl]

“DRL-17822” refers to CAS 1454689-50-9, and was developed by Dr. Reddy's Laboratories, and disclosed in WO 2014128564 and WO 2014076568. DRL-17822 has the structure:

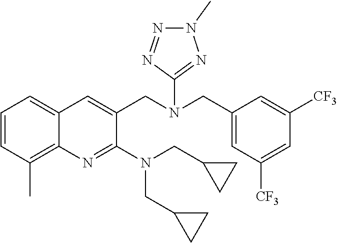 [SMILES: Cc1cccc2cc(CN(Cc3cc(C(F)(F)F)cc(C(F)(F)F)c3)c3nnn(C)n3)c(N(CC3CC3)CC3CC3)nc12]

“JNJ-28545595” refers to 1,1,1-Trifluoro-3-[2-[3-(1,1,2,2-tetra-fluoroethoxy)phenyl]-5-(3-trifluoromethoxyphenyl)-3,4-dihydro-2H-quinolin-1-yl]-propan-2-ol.

“JNJ-28614872” refers to 1,1,1-Trifluoro-3-[3-[3-(1,1,2,2-tetrafluoro-ethoxy)-phenyl]-8-(3-trifluoromethoxy-phenyl)-2,3-dihydro-benzo[1,4]oxazin-4-yl]-propan-2-ol.

The structure of JNJ-28545595 and JNJ-28614872 is set forth below:

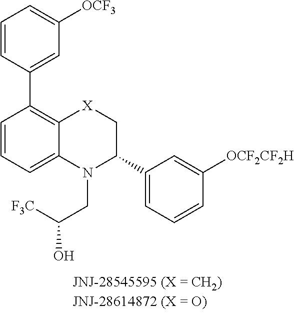

The structure of “BAY 19-4789” and “BAY 38-1315” is set forth below:

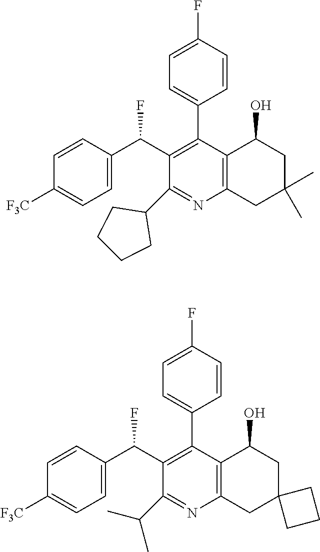 [SMILES: CC1(C)Cc2nc(C3CCCC3)c([C@@H](F)c3ccc(C(F)(F)F)cc3)c(-c3ccc(F)cc3)c2[C@@H](O)C1 ; CC(C)c1nc2c(c(-c3ccc(F)cc3)c1[C@@H](F)c1ccc(C(F)(F)F)cc1)[C@@H](O)CC1(CCC1)C2]

Additional CETP inhibitors useful in the compositions and methods of the invention include those disclosed in WO 2016/086453 or Chen et al., European Journal of Medicinal Chemistry, (2017) 139:201-213, and have the structure:

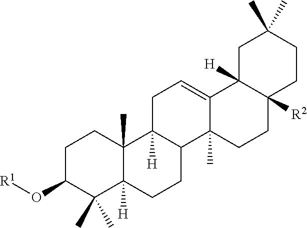

| R¹ | R² |
|---|---|
| H | —CO₂H |
| —COCH₃ | —CO₂H |
| —COCH₂CH₃ | —CO₂H |
| —CO(CH₂)₂CH₃ | —CO₂H |
| —CO(CH₂)₇CH₃ | —CO₂H |
| —CO(CH₂)₁₄CH₃ | —CO₂H |
| 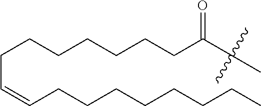 [SMILES: *C(=O)CCCCCCC/C=C\CCCCCCCC] | —CO₂H |
| 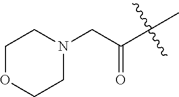 [SMILES: *C(=O)CN1CCOCC1] | —CO₂H |
| 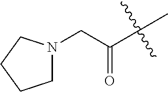 [SMILES: *C(=O)CN1CCCC1] | —CO₂H |
| 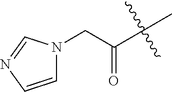 [SMILES: *C(=O)Cn1ccnc1] | —CO₂H |
| 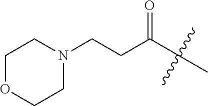 [SMILES: *C(=O)CCN1CCOCC1] | —CO₂H |
| 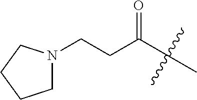 [SMILES: *C(=O)CCN1CCCC1] | —CO₂H |
| 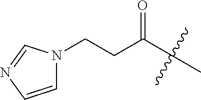 [SMILES: *C(=O)CCn1ccnc1] | —CO₂H |
| —CO(CH₂)₂CO₂H | —CH₃ |
| —CO(CH₂)₃CO₂H | —CH₃ |
| —CO(CH₂)₂CO₂H | —CO₂H |
| —CO(CH₂)₃CO₂H | —CO₂H |
| —CO(CH₂)₄CO₂H | —CO₂H |
| 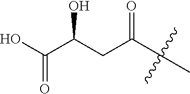 [SMILES: *C(=O)C[C@H](O)C(=O)O] | —CO₂H |
| —CO(CH₂)₂CONH₂ | —CO₂H |
| —CO(CH₂)₂CON(CH₃)₂ | —CO₂H |
| 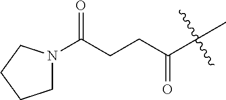 [SMILES: *C(=O)CCC(=O)N1CCCC1] | —CO₂H |
| 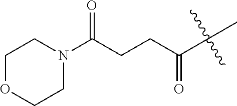 [SMILES: *C(=O)CCC(=O)N1CCOCC1] | —CO₂H |
| 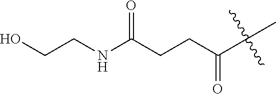 [SMILES: *C(=O)CCC(=O)NCCO] | —CO₂H |
| 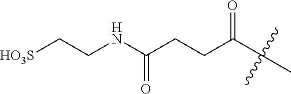 [SMILES: *C(=O)CCC(=O)NCCS(=O)(=O)O] | —CO₂H |
| —CO(CH₂)₃CONH₂ | —CO₂H |
| —CO(CH₂)₃CON(CH₃)₂ | —CO₂H |
| 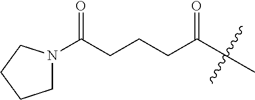 [SMILES: *C(=O)CCCC(=O)N1CCCC1] | —CO₂H |
| 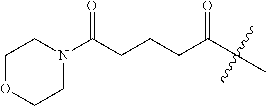 [SMILES: *C(=O)CCCC(=O)N1CCOCC1] | —CO₂H |
| 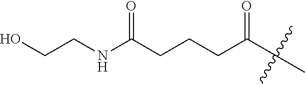 [SMILES: *C(=O)CCCC(=O)NCCO] | —CO₂H |
| 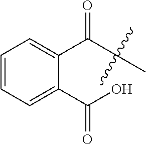 [SMILES: *C(=O)c1ccccc1C(=O)O] | —CO₂H |
| —CO(CH₂)₃CO₂H | —CO₂CH₂CO₂H |
| —CO(CH₂)₃CO₂H | —CO₂CH₃ |
| H | —CONH₂ |
| H | —CO₂CH₂CO₂H |

and pharmaceutically acceptable salts of the foregoing;

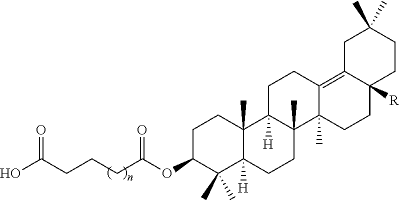

| n | R |
|---|---|
| 0 | —CO₂H |
| 0 | —CO₂CH₃ |
| 1 | —CO₂H |
| 1 | —CO₂CH₃ |
| 2 | —CO₂H |
| 2 | —CO₂CH₃ |

and pharmaceutically acceptable salts of the foregoing;

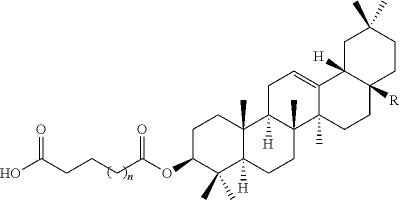

| n | R |
|---|---|
| 0 | —CONH₂ |
| 0 | —CON(CH₃)₂ |
| 1 | —CONH₂ |
| 1 | —CON(CH₃)₂ |
| 1 | —CONHCH₂CO₂H |
| 1 | —CONHCH₂CO₂CH₃ |
| 1 | —COCH₂CO₂H |
| 2 | —CONH₂ |
| 2 | —CON(CH₃)₂ |
| 2 | —CONHCH₂CO₂H |
| 2 | —CONHCH₂CO₂CH₃ |

and pharmaceutically acceptable salts of the foregoing;

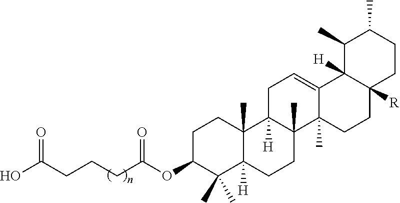

| n | R |
|---|---|
| 1 | —CO₂H |
| 1 | —CO₂CH₃ |
| 2 | —CO₂H |
| 2 | —CO₂CH₃ |
| 0 | —CON(CH₃)₂ |

and pharmaceutically acceptable salts of the foregoing;

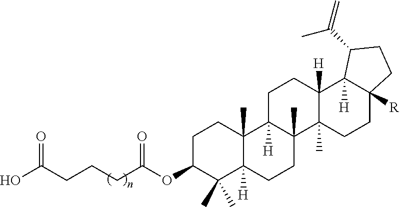

| n | R |
|---|---|
| 0 | —CON(CH₃)₂ |
| 0 | —CONH₂ |
| 1 | —CO₂H |
| 2 | —CO₂H |
| 2 | —CO₂CH₃ |

and pharmaceutically acceptable salts of the foregoing;

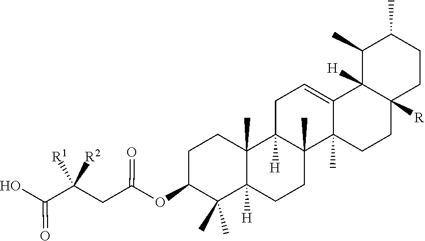

| R¹ | R² | R |
|---|---|---|
| H | OH | CH₃ |
| CH₃ | CH₃ | CH₃ |
| H | OH | —CO₂H |
| H | OH | —CONH₂ |
| CH₃ | CH₃ | —CONH₂ |
| H | OH | —CON(CH₃)₂ |
| CH₃ | CH₃ | —CON(CH₃)₂ |

and pharmaceutically acceptable salts of the foregoing;

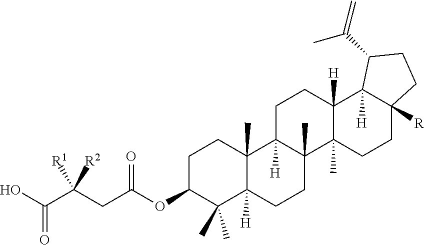

| R1 | R2 | R |
|---|---|---|
| H | OH | CH₃ |
| H | OH | —CO₂H |
| H | OH | —CONH₂ |
| CH₃ | CH₃ | —CONH₂ |
| H | OH | —CON(CH₃)₂ |
| CH₃ | CH₃ | —CON(CH₃)₂ |

and pharmaceutically acceptable salts of the foregoing;

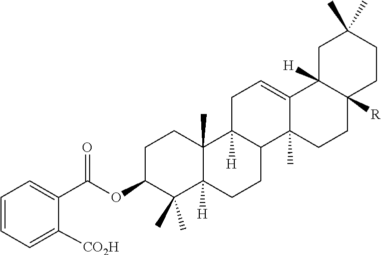

R
—CO₂CH₃
—CONH₂
—CON(CH₃)₂
—CONHCH₂CO₂H
—CONHCH₂CO₂CH₃

and pharmaceutically acceptable salts of the foregoing; and

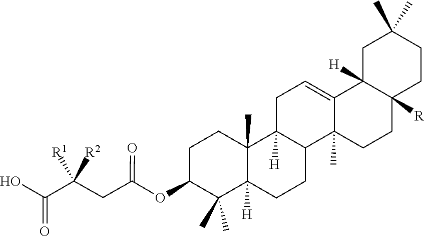

| R¹ | R² | R |
|---|---|---|
| H | OH | CH₃ |
| CH₃ | CH₃ | CH₃ |
| H | OH | —CONH₂ |
| CH₃ | CH₃ | —CONH₂ |
| H | OH | —CON(CH₃)₂ |
| CH₃ | CH₃ | —CON(CH₃)₂ |

and pharmaceutically acceptable salts of the foregoing.

Additional CETP inhibitors useful in the compositions and methods of the invention are disclosed in WO 2016/086453 or Chen et al. and include, but are not limited to:

Structure

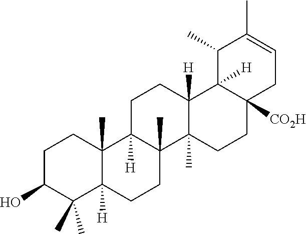

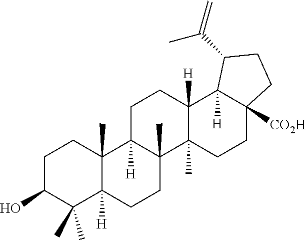

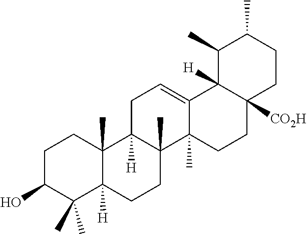

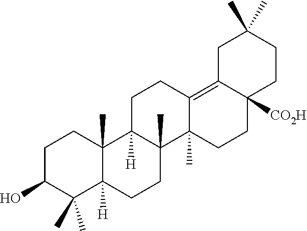

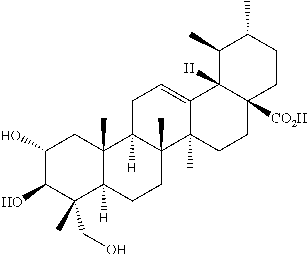

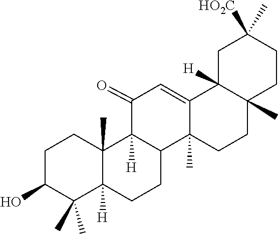

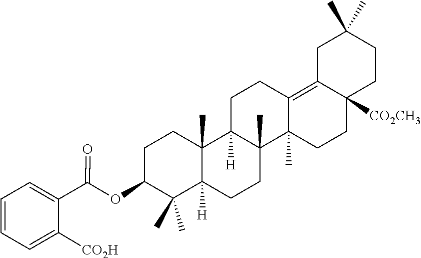

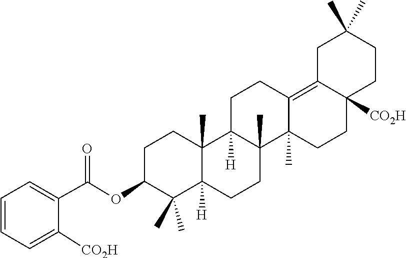

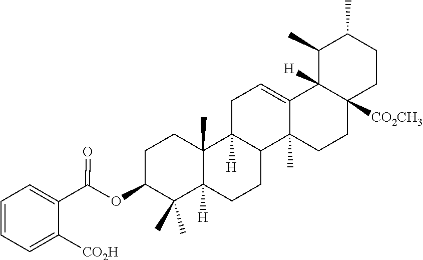

and pharmaceutically acceptable salts of the foregoing.

Further CETP inhibitors useful in the compositions and methods of the invention include those disclosed in WO 2017/011279, and have the structure:

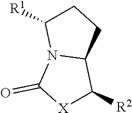

| X | R¹ | R² |
|---|---|---|
| S | 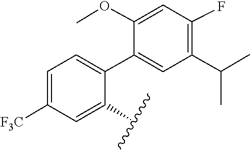 | 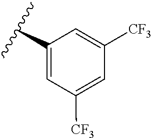 |
| S | 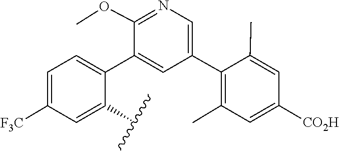 | 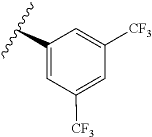 |
| S | 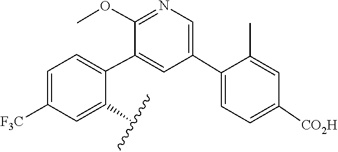 | 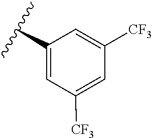 |
| S | 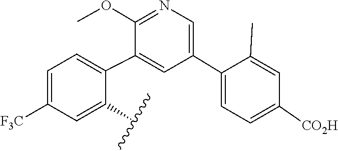 | 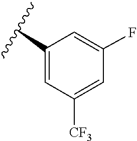 |
| S | 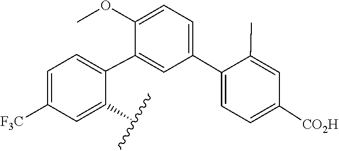 | 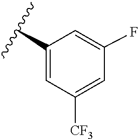 |
| S | 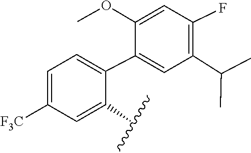 | 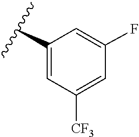 |
| S | 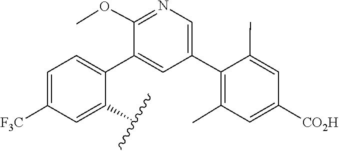 | 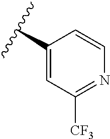 |
| S | 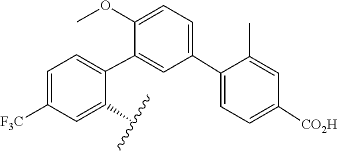 | 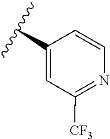 |
| S | 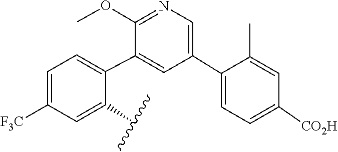 | 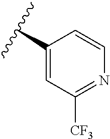 |
| S | 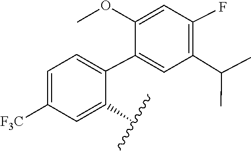 | 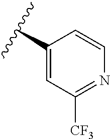 |
| S | 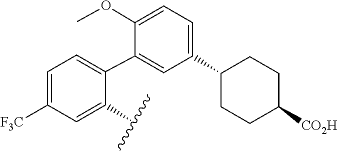 | 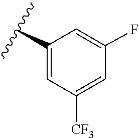 |
| S | 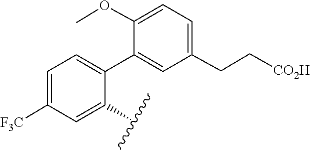 | 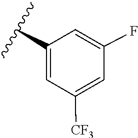 |
| CH₂ | 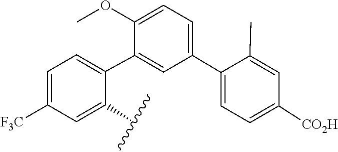 | 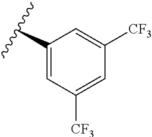 |
| CH₂ | 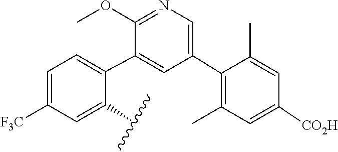 | 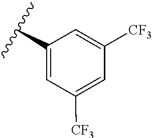 |
| CH₂ | 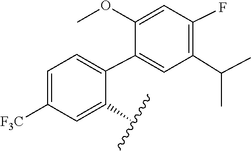 | 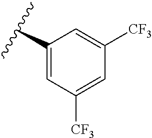 |
| CH₂ | 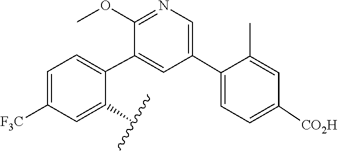 | 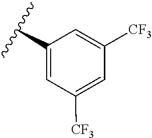 |
| CH₂ | 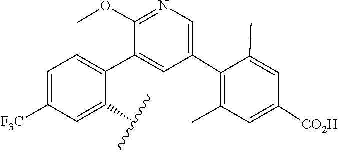 | 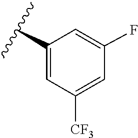 |
| CH₂ | 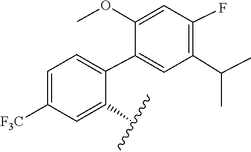 | 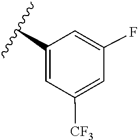 |
| CH₂ | 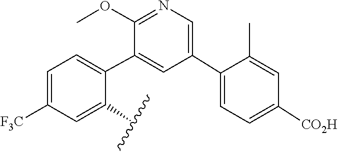 | 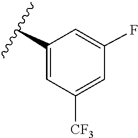 |
| CH₂ | 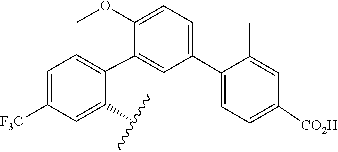 | 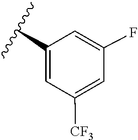 |
| CH₂ | 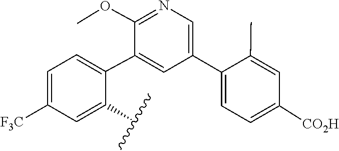 | 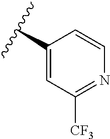 |

and pharmaceutically acceptable salts of the foregoing.

Still other CETP inhibitors useful in the compositions and methods of the invention include those disclosed in WO2016/018729, and have a structure according to the following:

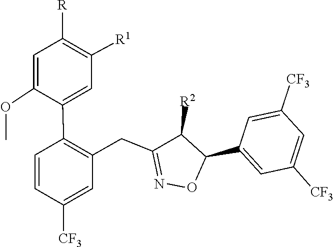

| R | R¹ | R² |
|---|---|---|
| F | 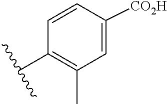 [SMILES: *c1ccc(C(=O)O)cc1C] | H |
| F | 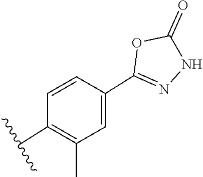 [SMILES: *c1ccc(-c2n[nH]c(=O)o2)cc1C] | H |
| H | 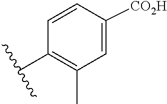 [SMILES: *c1ccc(C(=O)O)cc1C] | CH₃ |
| F | 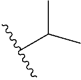 [SMILES: *C(C)C] | H |
| H | 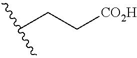 [SMILES: *CCC(=O)O] | H |

and pharmaceutically acceptable salts of the foregoing;

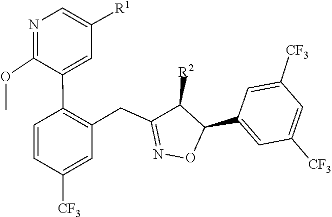

| R¹ | R² |
|---|---|
| 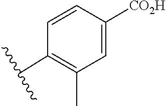 [SMILES: *c1ccc(C(=O)O)cc1C] | H |
| 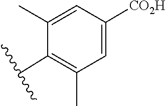 [SMILES: *c1c(C)cc(C(=O)O)cc1C] | CH₃ |
| 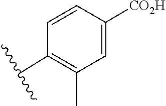 [SMILES: *c1ccc(C(=O)O)cc1C] | CH₃ |

and pharmaceutically acceptable salts of the foregoing;

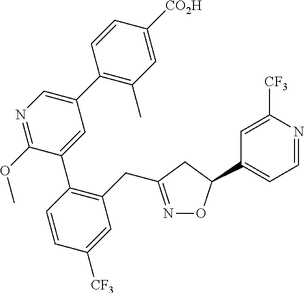 [SMILES: COc1ncc(-c2ccc(C(=O)O)cc2C)cc1-c1ccc(C(F)(F)F)cc1CC1=NO[C@H](c2ccnc(C(F)(F)F)c2)C1]

and pharmaceutically acceptable salts thereof;

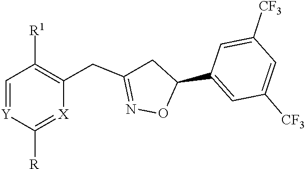

| X | Y | R | R¹ |
|---|---|---|---|
| CH | N | CF₃ | 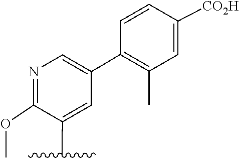 [SMILES: *c1cc(-c2ccc(C(=O)O)cc2C)cnc1OC] |
| CH | N | CF₃ | 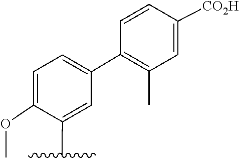 [SMILES: *c1cc(-c2ccc(C(=O)O)cc2C)ccc1OC] |
| N | CH | CF₃ | 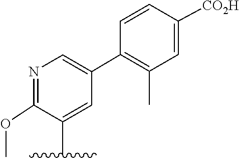 [SMILES: *c1cc(-c2ccc(C(=O)O)cc2C)cnc1OC] |
| N | CH | OCH₃ | 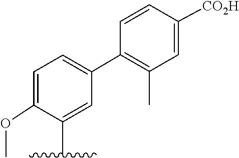 [SMILES: *c1cc(-c2ccc(C(=O)O)cc2C)ccc1OC] |
| N | CH | OCH₃ | 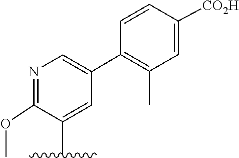 [SMILES: *c1cc(-c2ccc(C(=O)O)cc2C)cnc1OC] |

and pharmaceutically acceptable salts of the foregoing; and

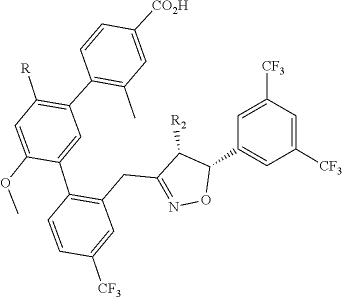

| R | R² |
|---|---|
| F | H |
| H | CH₃ |

and pharmaceutically acceptable salts of the foregoing.

Additional CETP inhibitors useful in the compositions and methods of the invention are disclosed in U.S. Pat. No. 7,781,426, including, but not limited to:

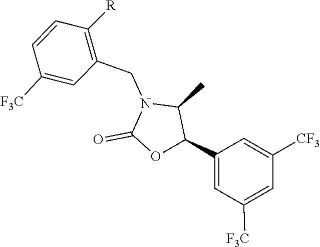

R

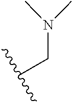 [SMILES: *CN(C)C]

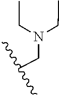 [SMILES: *CN(CC)CC]

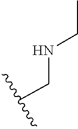 [SMILES: *CNCC]

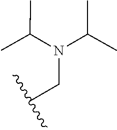 [SMILES: *CN(C(C)C)C(C)C]

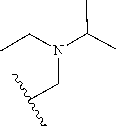 [SMILES: *CN(CC)C(C)C]

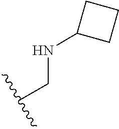 [SMILES: *CNC1CCC1]

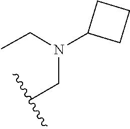 [SMILES: *CN(CC)C1CCC1]

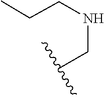 [SMILES: *CNCCC]

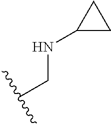 [SMILES: *CNC1CC1]

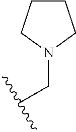 [SMILES: *CN1CCCC1]

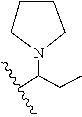 [SMILES: *C(CC)N1CCCC1]

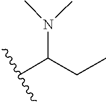 [SMILES: *C(CC)N(C)C]

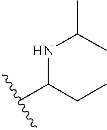 [SMILES: *C(CC)NC(C)C]

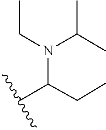 [SMILES: *C(CC)N(CC)C(C)C]

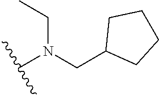 [SMILES: *N(CC)CC1CCCC1]

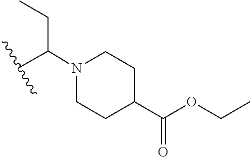 [SMILES: *C(CC)N1CCC(C(=O)OCC)CC1]

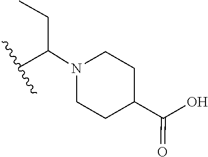 [SMILES: *C(CC)N1CCC(C(=O)O)CC1]

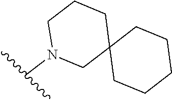 [SMILES: *N1CCCC2(CCCCC2)C1]

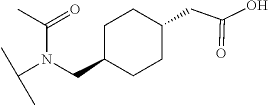 [SMILES: CC(=O)N(C[C@H]1CC[C@H](CC(=O)O)CC1)C(C)C]

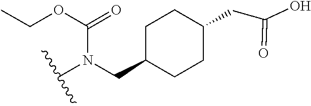 [SMILES: *N(C[C@H]1CC[C@H](CC(=O)O)CC1)C(=O)OCC]

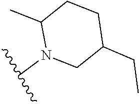 [SMILES: *N1CC(CC)CCC1C]

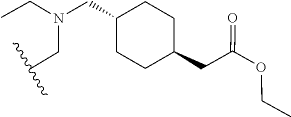 [SMILES: *CN(CC)C[C@H]1CC[C@H](CC(=O)OCC)CC1]

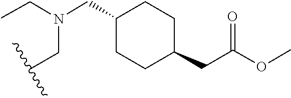 [SMILES: *CN(CC)C[C@H]1CC[C@H](CC(=O)OC)CC1]

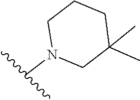 [SMILES: *N1CCCC(C)(C)C1]

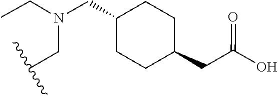 [SMILES: *CN(CC)C[C@H]1CC[C@H](CC(=O)O)CC1]

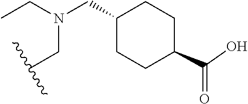 [SMILES: *CN(CC)C[C@H]1CC[C@H](C(=O)O)CC1]

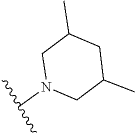 [SMILES: *N1CC(C)CC(C)C1]

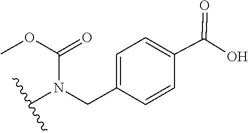 [SMILES: *N(Cc1ccc(C(=O)O)cc1)C(=O)OC]

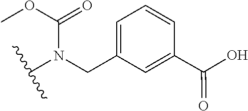 [SMILES: *N(Cc1cccc(C(=O)O)c1)C(=O)OC]

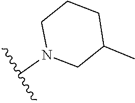 [SMILES: *N1CCCC(C)C1]

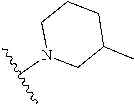 [SMILES: *N1CCCC(C)C1]

and pharmaceutically acceptable salts of the foregoing;

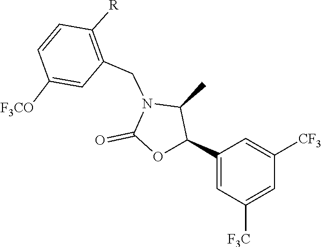

R

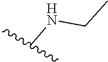 [SMILES: *NCC]

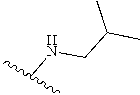 [SMILES: *NCC(C)C]

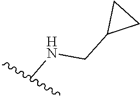 [SMILES: *NCC1CC1]

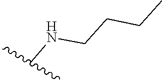 [SMILES: *NCCCC]

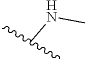 [SMILES: *NC]

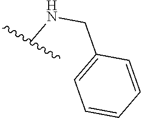 [SMILES: *NCc1ccccc1]

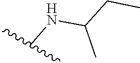 [SMILES: *NC(C)CC]

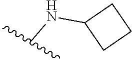 [SMILES: *NC1CCC1]

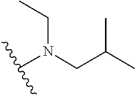 [SMILES: *N(CC)CC(C)C]

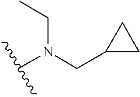 [SMILES: *N(CC)CC1CC1]

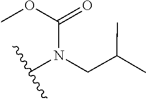 [SMILES: *N(CC(C)C)C(=O)OC]

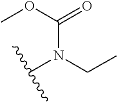 [SMILES: *N(CC)C(=O)OC]

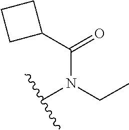 [SMILES: *N(CC)C(=O)C1CCC1]

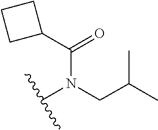 [SMILES: *N(CC(C)C)C(=O)C1CCC1]

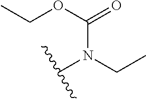 [SMILES: *N(CC)C(=O)OCC]

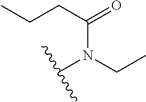 [SMILES: *N(CC)C(=O)CCC]

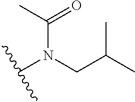 [SMILES: *N(CC(C)C)C(C)=O]

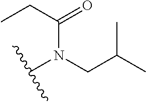 [SMILES: *N(CC(C)C)C(=O)CC]

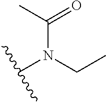 [SMILES: *N(CC)C(C)=O]

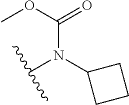 [SMILES: *N(C(=O)OC)C1CCC1]

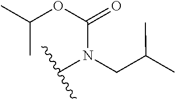 [SMILES: *N(CC(C)C)C(=O)OC(C)C]

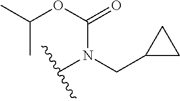 [SMILES: *N(CC1CC1)C(=O)OC(C)C]

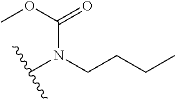 [SMILES: *N(CCCC)C(=O)OC]

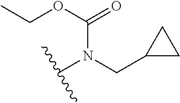 [SMILES: *N(CC1CC1)C(=O)OCC]

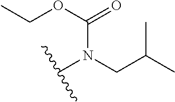 [SMILES: *N(CC(C)C)C(=O)OCC]

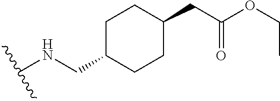 [SMILES: *NC[C@H]1CC[C@H](CC(=O)OCC)CC1]

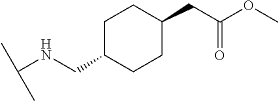 [SMILES: COC(=O)C[C@H]1CC[C@H](CNC(C)C)CC1]

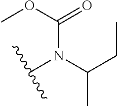 [SMILES: *N(C(=O)OC)C(C)CC]

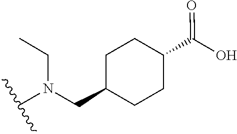 [SMILES: *N(CC)C[C@H]1CC[C@H](C(=O)O)CC1]

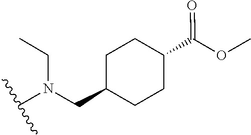 [SMILES: *N(CC)C[C@H]1CC[C@H](C(=O)OC)CC1]

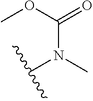

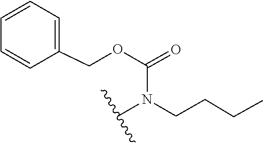 [SMILES: *N(CCCC)C(=O)OCc1ccccc1]

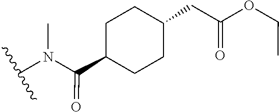 [SMILES: *N(C)C(=O)[C@H]1CC[C@H](CC(=O)OCC)CC1]

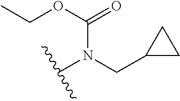 [SMILES: *N(CC1CC1)C(=O)OCC]

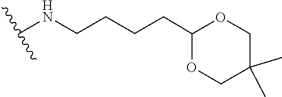 [SMILES: *NCCCCC1OCC(C)(C)CO1]

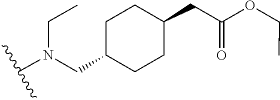 [SMILES: *N(CC)C[C@H]1CC[C@H](CC(=O)OCC)CC1]

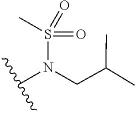 [SMILES: *N(CC(C)C)S(C)(=O)=O]

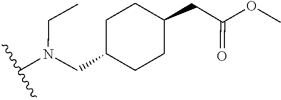 [SMILES: *N(CC)C[C@H]1CC[C@H](CC(=O)OC)CC1]

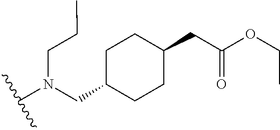 [SMILES: *N(CCC)C[C@H]1CC[C@H](CC(=O)OCC)CC1]

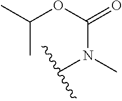 [SMILES: *N(C)C(=O)OC(C)C]

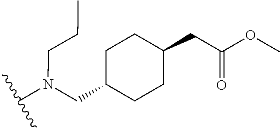 [SMILES: *N(CCC)C[C@H]1CC[C@H](CC(=O)OC)CC1]

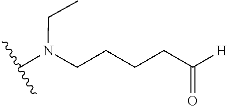 [SMILES: *N(CC)CCCCC=O]

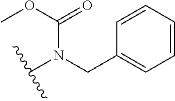 [SMILES: *N(Cc1ccccc1)C(=O)OC]

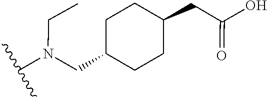 [SMILES: *N(CC)C[C@H]1CC[C@H](CC(=O)O)CC1]

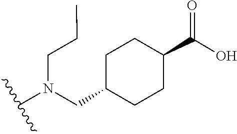 [SMILES: *N(CCC)C[C@H]1CC[C@H](C(=O)O)CC1]

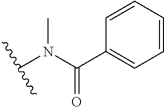 [SMILES: *N(C)C(=O)c1ccccc1]

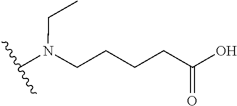 [SMILES: *N(CC)CCCCC(=O)O]

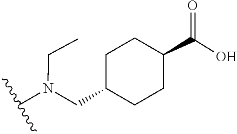 [SMILES: *N(CC)C[C@H]1CC[C@H](C(=O)O)CC1]

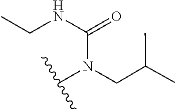 [SMILES: *N(CC(C)C)C(=O)NCC]

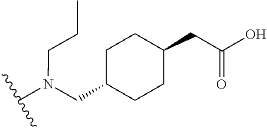 [SMILES: *N(CCC)C[C@H]1CC[C@H](CC(=O)O)CC1]

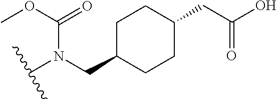 [SMILES: *N(C[C@H]1CC[C@H](CC(=O)O)CC1)C(=O)OC]

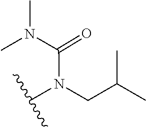 [SMILES: *N(CC(C)C)C(=O)N(C)C]

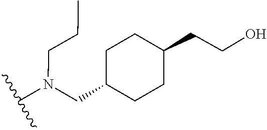 [SMILES: *N(CCC)C[C@H]1CC[C@H](CCO)CC1]

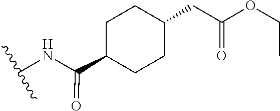 [SMILES: *NC(=O)[C@H]1CC[C@H](CC(=O)OCC)CC1]

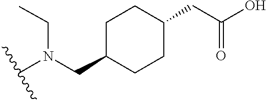 [SMILES: *N(CC)C[C@H]1CC[C@H](CC(=O)O)CC1]

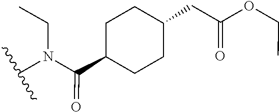 [SMILES: *N(CC)C(=O)[C@H]1CC[C@H](CC(=O)OCC)CC1]

and pharmaceutically acceptable salts of the foregoing;

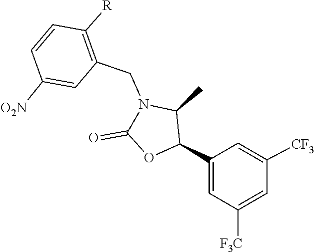

R

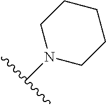 [SMILES: *N1CCCCC1]

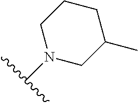 [SMILES: *N1CCCC(C)C1]

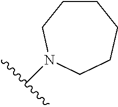 [SMILES: *N1CCCCCC1]

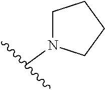 [SMILES: *N1CCCC1]

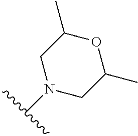 [SMILES: *N1CC(C)OC(C)C1]

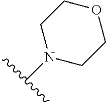 [SMILES: *N1CCOCC1]

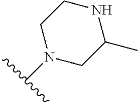 [SMILES: *N1CCNC(C)C1]

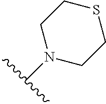 [SMILES: *N1CCSCC1]

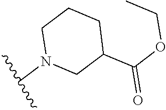 [SMILES: *N1CCCC(C(=O)OCC)C1]

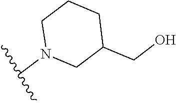 [SMILES: *N1CCCC(CO)C1]

and pharmaceutically acceptable salts of the foregoing; and

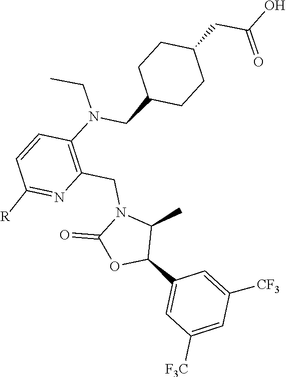

R
H

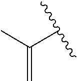 [SMILES: *C(=C)C]

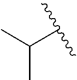 [SMILES: *C(C)C]

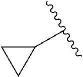 [SMILES: *C1CC1]

and pharmaceutically acceptable salts of the foregoing.

Additional CETP inhibitors useful in the compositions and methods of the invention are disclosed in U.S. Pat. No. 7,652,049, including, but not limited to:

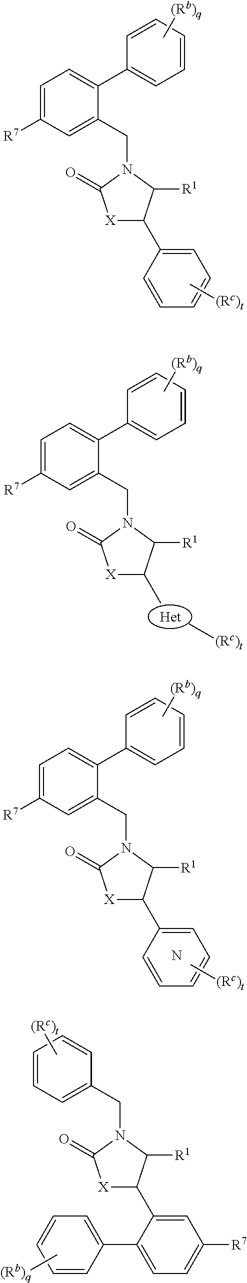

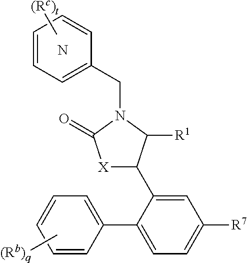

and pharmaceutically acceptable salts of the foregoing;

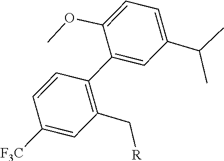

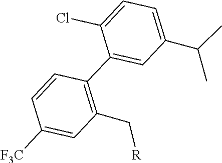

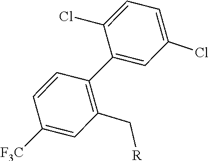

R

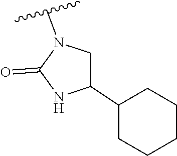 [SMILES: *N1CC(C2CCCCC2)NC1=O]

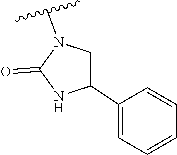 [SMILES: *N1CC(c2ccccc2)NC1=O]

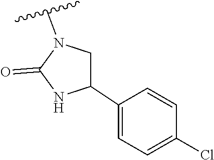 [SMILES: *N1CC(c2ccc(Cl)cc2)NC1=O]

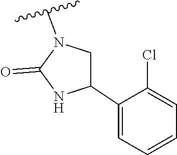 [SMILES: *N1CC(c2ccccc2Cl)NC1=O]

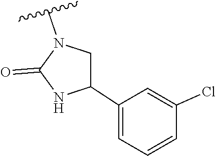 [SMILES: *N1CC(c2cccc(Cl)c2)NC1=O]

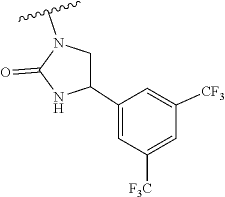 [SMILES: *N1CC(c2cc(C(F)(F)F)cc(C(F)(F)F)c2)NC1=O]

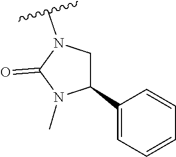 [SMILES: *N1C[C@@H](c2ccccc2)N(C)C1=O]

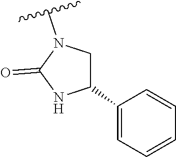

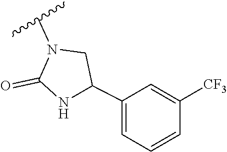 [SMILES: *N1CC(c2cccc(C(F)(F)F)c2)NC1=O]

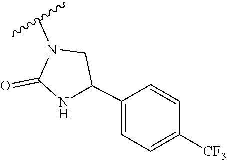 [SMILES: *N1CC(c2ccc(C(F)(F)F)cc2)NC1=O]

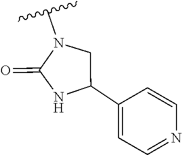 [SMILES: *N1CC(c2ccncc2)NC1=O]

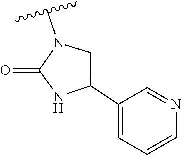 [SMILES: *N1CC(c2cccnc2)NC1=O]

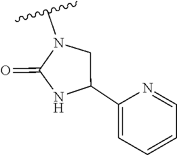 [SMILES: *N1CC(c2ccccn2)NC1=O]

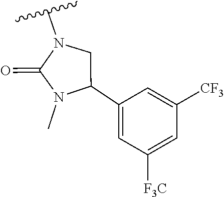 [SMILES: *N1CC(c2cc(C(F)(F)F)cc(C(F)(F)F)c2)N(C)C1=O]

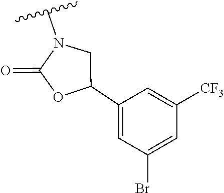 [SMILES: *N1CC(c2cc(Br)cc(C(F)(F)F)c2)OC1=O]

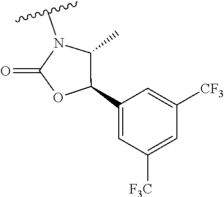 [SMILES: *N1C(=O)O[C@H](c2cc(C(F)(F)F)cc(C(F)(F)F)c2)[C@H]1C]

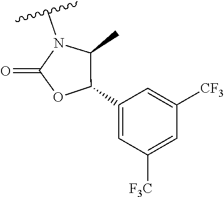 [SMILES: *N1C(=O)O[C@@H](c2cc(C(F)(F)F)cc(C(F)(F)F)c2)[C@@H]1C]

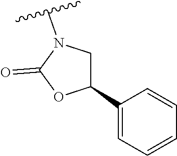 [SMILES: *N1C[C@@H](c2ccccc2)OC1=O]

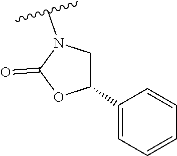 [SMILES: *N1C[C@H](c2ccccc2)OC1=O]

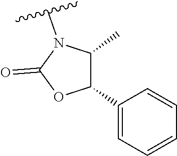 [SMILES: *N1C(=O)O[C@@H](c2ccccc2)[C@H]1C]

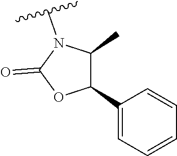 [SMILES: *N1C(=O)O[C@H](c2ccccc2)[C@@H]1C]

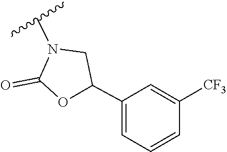 [SMILES: *N1CC(c2cccc(C(F)(F)F)c2)OC1=O]

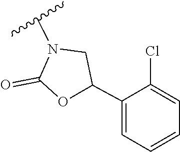 [SMILES: *N1CC(c2ccccc2Cl)OC1=O]

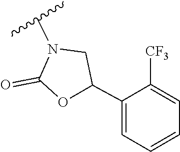 [SMILES: *N1CC(c2ccccc2C(F)(F)F)OC1=O]

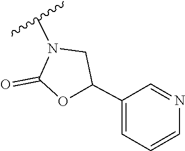 [SMILES: *N1CC(c2cccnc2)OC1=O]

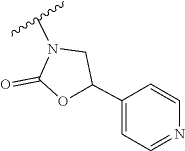 [SMILES: *N1CC(c2ccncc2)OC1=O]

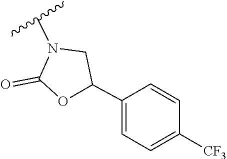 [SMILES: *N1CC(c2ccc(C(F)(F)F)cc2)OC1=O]

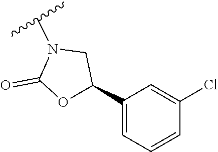 [SMILES: *N1C[C@@H](c2cccc(Cl)c2)OC1=O]

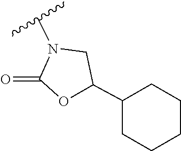 [SMILES: *N1CC(C2CCCCC2)OC1=O]

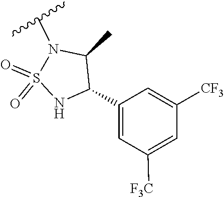 [SMILES: *N1[C@@H](C)[C@H](c2cc(C(F)(F)F)cc(C(F)(F)F)c2)NS1(=O)=O]

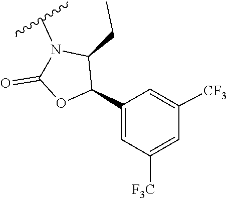 [SMILES: *N1C(=O)O[C@H](c2cc(C(F)(F)F)cc(C(F)(F)F)c2)[C@@H]1CC]

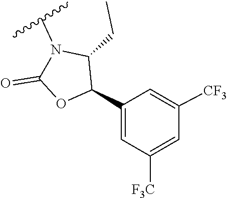 [SMILES: *N1C(=O)O[C@H](c2cc(C(F)(F)F)cc(C(F)(F)F)c2)[C@H]1CC]

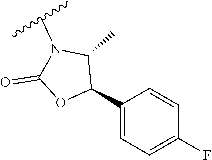 [SMILES: *N1C(=O)O[C@H](c2ccc(F)cc2)[C@H]1C]

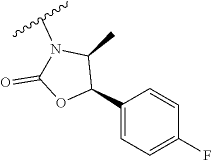 [SMILES: *N1C(=O)O[C@H](c2ccc(F)cc2)[C@@H]1C]

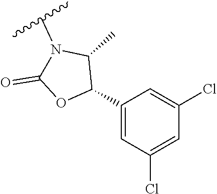 [SMILES: *N1C(=O)O[C@@H](c2cc(Cl)cc(Cl)c2)[C@H]1C]

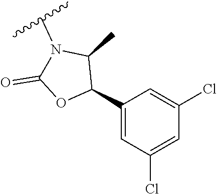 [SMILES: *N1C(=O)O[C@H](c2cc(Cl)cc(Cl)c2)[C@@H]1C]

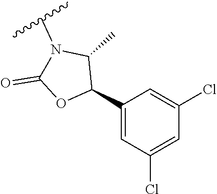 [SMILES: *N1C(=O)O[C@H](c2cc(Cl)cc(Cl)c2)[C@H]1C]

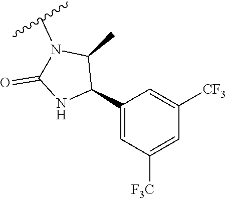 [SMILES: *N1C(=O)N[C@H](c2cc(C(F)(F)F)cc(C(F)(F)F)c2)[C@@H]1C]

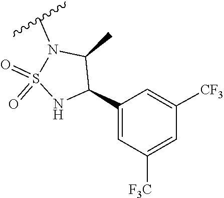 [SMILES: *N1[C@@H](C)[C@@H](c2cc(C(F)(F)F)cc(C(F)(F)F)c2)NS1(=O)=O]

and pharmaceutically acceptable salts of the foregoing;

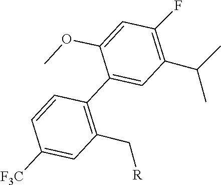

R

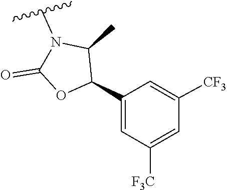 [SMILES: *N1C(=O)O[C@H](c2cc(C(F)(F)F)cc(C(F)(F)F)c2)[C@@H]1C]

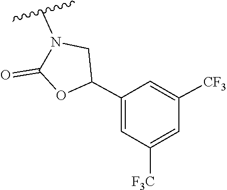 [SMILES: *N1CC(c2cc(C(F)(F)F)cc(C(F)(F)F)c2)OC1=O]

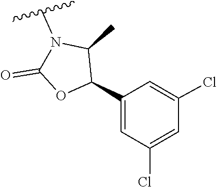 [SMILES: *N1C(=O)O[C@H](c2cc(Cl)cc(Cl)c2)[C@@H]1C]

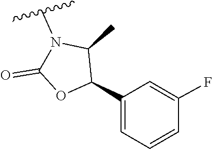 [SMILES: *N1C(=O)O[C@H](c2cccc(F)c2)[C@@H]1C]

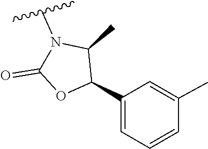 [SMILES: *N1C(=O)O[C@H](c2cccc(C)c2)[C@@H]1C]

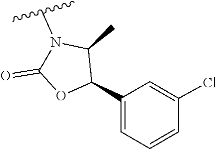 [SMILES: *N1C(=O)O[C@H](c2cccc(Cl)c2)[C@@H]1C]

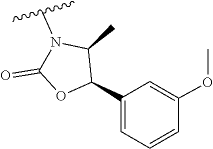 [SMILES: *N1C(=O)O[C@H](c2cccc(OC)c2)[C@@H]1C]

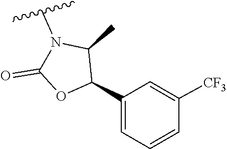 [SMILES: *N1C(=O)O[C@H](c2cccc(C(F)(F)F)c2)[C@@H]1C]

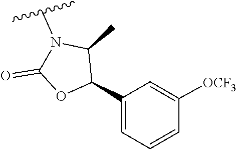 [SMILES: *N1C(=O)O[C@H](c2cccc(OC(F)(F)F)c2)[C@@H]1C]

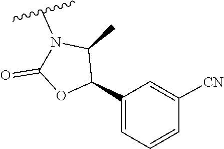 [SMILES: *N1C(=O)O[C@H](c2cccc(C#N)c2)[C@@H]1C]

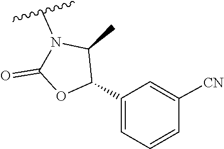 [SMILES: *N1C(=O)O[C@@H](c2cccc(C#N)c2)[C@@H]1C]

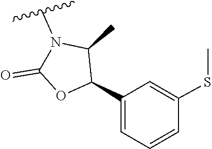 [SMILES: *N1C(=O)O[C@H](c2cccc(SC)c2)[C@@H]1C]

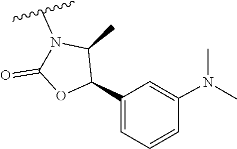 [SMILES: *N1C(=O)O[C@H](c2cccc(N(C)C)c2)[C@@H]1C]

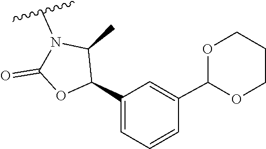 [SMILES: *N1C(=O)O[C@H](c2cccc(C3OCCCO3)c2)[C@@H]1C]

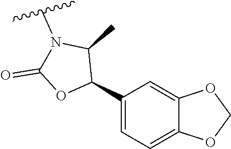 [SMILES: *N1C(=O)O[C@H](c2ccc3c(c2)OCO3)[C@@H]1C]

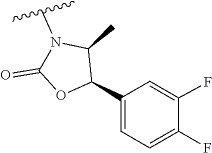 [SMILES: *N1C(=O)O[C@H](c2ccc(F)c(F)c2)[C@@H]1C]

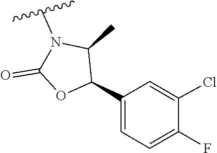 [SMILES: *N1C(=O)O[C@H](c2ccc(F)c(Cl)c2)[C@@H]1C]

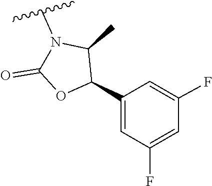 [SMILES: *N1C(=O)O[C@H](c2cc(F)cc(F)c2)[C@@H]1C]

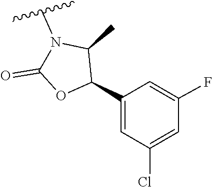 [SMILES: *N1C(=O)O[C@H](c2cc(F)cc(Cl)c2)[C@@H]1C]

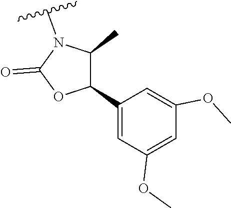 [SMILES: *N1C(=O)O[C@H](c2cc(OC)cc(OC)c2)[C@@H]1C]

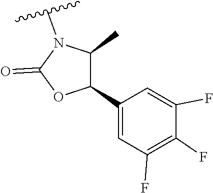 [SMILES: *N1C(=O)O[C@H](c2cc(F)c(F)c(F)c2)[C@@H]1C]

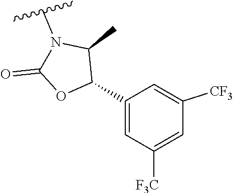 [SMILES: *N1C(=O)O[C@@H](c2cc(C(F)(F)F)cc(C(F)(F)F)c2)[C@@H]1C]

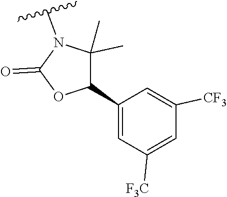

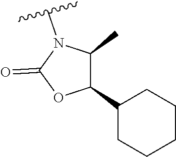 [SMILES: *N1C(=O)O[C@H](C2CCCCC2)[C@@H]1C]

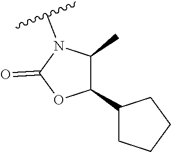 [SMILES: *N1C(=O)O[C@H](C2CCCC2)[C@@H]1C]

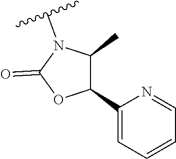 [SMILES: *N1C(=O)O[C@H](c2ccccn2)[C@@H]1C]

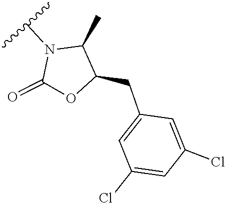 [SMILES: *N1C(=O)O[C@H](Cc2cc(Cl)cc(Cl)c2)[C@@H]1C]

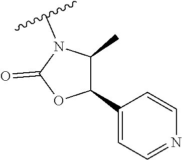 [SMILES: *N1C(=O)O[C@H](c2ccncc2)[C@@H]1C]

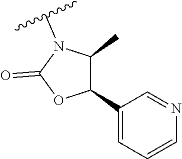 [SMILES: *N1C(=O)O[C@H](c2cccnc2)[C@@H]1C]

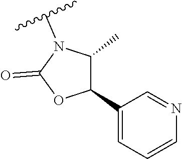 [SMILES: *N1C(=O)O[C@H](c2cccnc2)[C@H]1C]

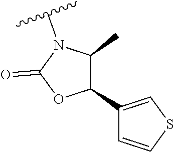 [SMILES: *N1C(=O)O[C@H](c2ccsc2)[C@@H]1C]

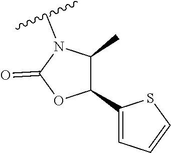 [SMILES: *N1C(=O)O[C@H](c2cccs2)[C@@H]1C]

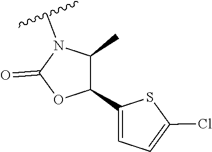 [SMILES: *N1C(=O)O[C@H](c2ccc(Cl)s2)[C@@H]1C]

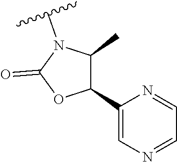 [SMILES: *N1C(=O)O[C@H](c2cnccn2)[C@@H]1C]

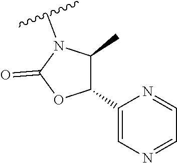 [SMILES: *N1C(=O)O[C@@H](c2cnccn2)[C@@H]1C]

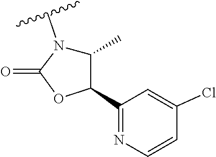

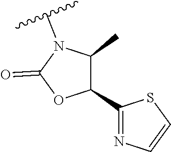 [SMILES: *N1C(=O)O[C@H](c2nccs2)[C@@H]1C]

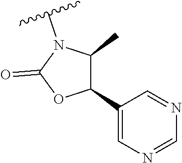 [SMILES: *N1C(=O)O[C@H](c2cncnc2)[C@@H]1C]

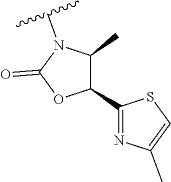 [SMILES: *N1C(=O)O[C@H](c2nc(C)cs2)[C@@H]1C]

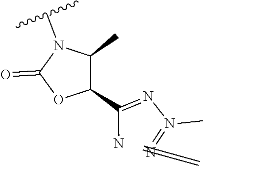

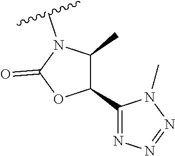 [SMILES: *N1C(=O)O[C@H](c2nnnn2C)[C@@H]1C]

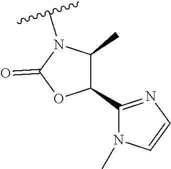 [SMILES: *N1C(=O)O[C@H](c2nccn2C)[C@@H]1C]

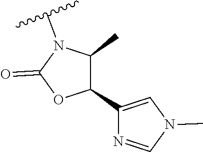 [SMILES: *N1C(=O)O[C@H](c2cn(C)cn2)[C@@H]1C]

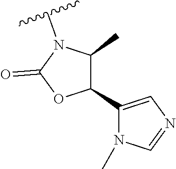 [SMILES: *N1C(=O)O[C@H](c2cncn2C)[C@@H]1C]

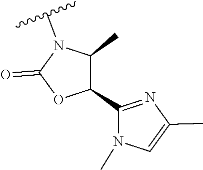 [SMILES: *N1C(=O)O[C@H](c2nc(C)cn2C)[C@@H]1C]

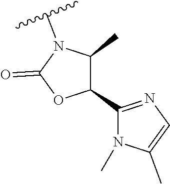 [SMILES: *N1C(=O)O[C@H](c2ncc(C)n2C)[C@@H]1C]

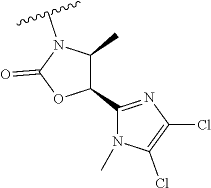 [SMILES: *N1C(=O)O[C@H](c2nc(Cl)c(Cl)n2C)[C@@H]1C]

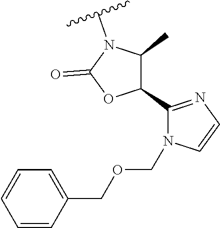 [SMILES: *N1C(=O)O[C@H](c2nccn2COCc2ccccc2)[C@@H]1C]

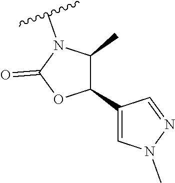 [SMILES: *N1C(=O)O[C@H](c2cnn(C)c2)[C@@H]1C]

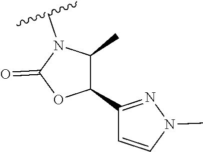 [SMILES: *N1C(=O)O[C@H](c2ccn(C)n2)[C@@H]1C]

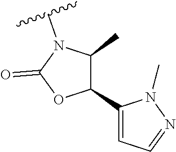 [SMILES: *N1C(=O)O[C@H](c2ccnn2C)[C@@H]1C]

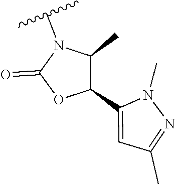

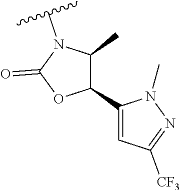 [SMILES: *N1C(=O)O[C@H](c2cc(C(F)(F)F)nn2C)[C@@H]1C]

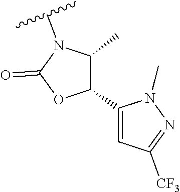 [SMILES: *N1C(=O)O[C@@H](c2cc(C(F)(F)F)nn2C)[C@H]1C]

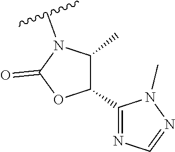 [SMILES: *N1C(=O)O[C@@H](c2ncnn2C)[C@H]1C]

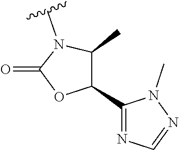

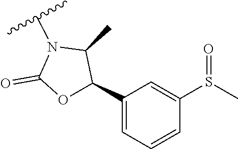 [SMILES: *N1C(=O)O[C@H](c2cccc(S(C)=O)c2)[C@@H]1C]

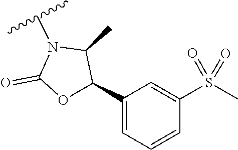 [SMILES: *N1C(=O)O[C@H](c2cccc(S(C)(=O)=O)c2)[C@@H]1C]

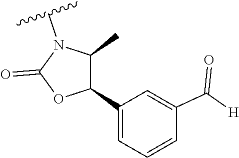 [SMILES: *N1C(=O)O[C@H](c2cccc(C=O)c2)[C@@H]1C]

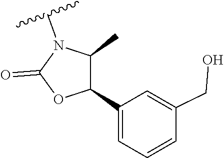 [SMILES: *N1C(=O)O[C@H](c2cccc(CO)c2)[C@@H]1C]

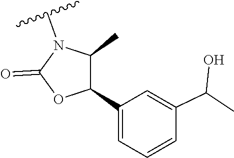 [SMILES: *N1C(=O)O[C@H](c2cccc(C(C)O)c2)[C@@H]1C]

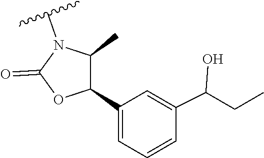 [SMILES: *N1C(=O)O[C@H](c2cccc(C(O)CC)c2)[C@@H]1C]

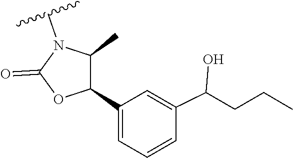 [SMILES: *N1C(=O)O[C@H](c2cccc(C(O)CCC)c2)[C@@H]1C]

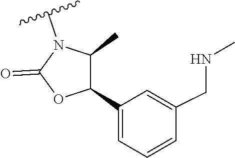 [SMILES: *N1C(=O)O[C@H](c2cccc(CNC)c2)[C@@H]1C]

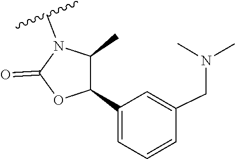 [SMILES: *N1C(=O)O[C@H](c2cccc(CN(C)C)c2)[C@@H]1C]

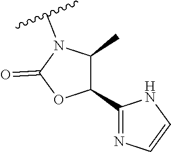 [SMILES: *N1C(=O)O[C@H](c2ncc[nH]2)[C@@H]1C]

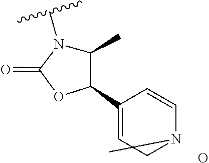

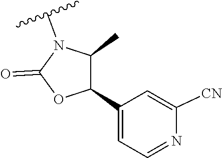 [SMILES: *N1C(=O)O[C@H](c2ccnc(C#N)c2)[C@@H]1C]

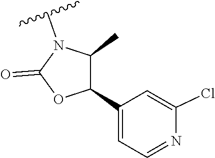 [SMILES: *N1C(=O)O[C@H](c2ccnc(Cl)c2)[C@@H]1C]

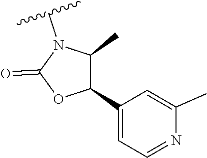 [SMILES: *N1C(=O)O[C@H](c2ccnc(C)c2)[C@@H]1C]

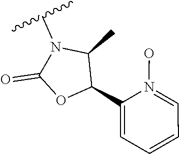

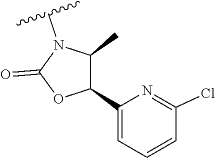 [SMILES: *N1C(=O)O[C@H](c2cccc(Cl)n2)[C@@H]1C]

and pharmaceutically acceptable salts of the foregoing;

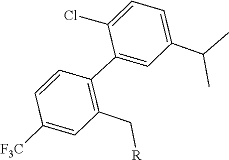

R

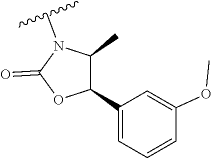 [SMILES: *N1C(=O)O[C@H](c2cccc(OC)c2)[C@@H]1C]

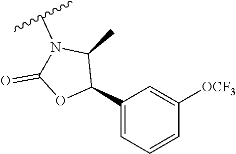 [SMILES: *N1C(=O)O[C@H](c2cccc(OC(F)(F)F)c2)[C@@H]1C]

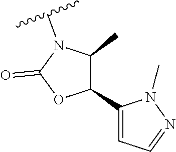 [SMILES: *N1C(=O)O[C@H](c2ccnn2C)[C@@H]1C]

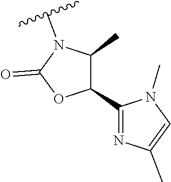 [SMILES: *N1C(=O)O[C@H](c2nc(C)cn2C)[C@@H]1C]

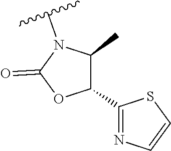 [SMILES: *N1C(=O)O[C@@H](c2nccs2)[C@@H]1C]

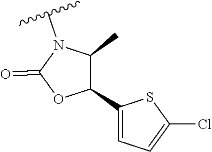 [SMILES: *N1C(=O)O[C@H](c2ccc(Cl)s2)[C@@H]1C]

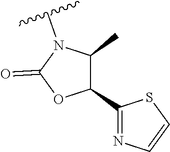 [SMILES: *N1C(=O)O[C@H](c2nccs2)[C@@H]1C]

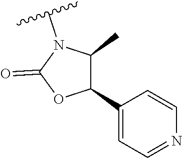 [SMILES: *N1C(=O)O[C@H](c2ccncc2)[C@@H]1C]

and pharmaceutically acceptable salts of the foregoing;

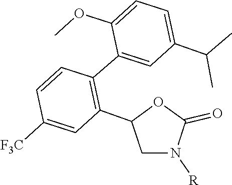

R

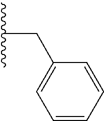 [SMILES: *Cc1ccccc1]

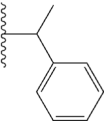 [SMILES: *C(C)c1ccccc1]

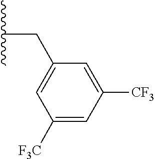 [SMILES: *Cc1cc(C(F)(F)F)cc(C(F)(F)F)c1]

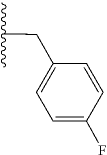 [SMILES: *Cc1ccc(F)cc1]

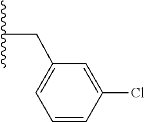 [SMILES: *Cc1cccc(Cl)c1]

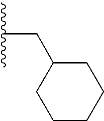 [SMILES: *CC1CCCCC1]

and pharmaceutically acceptable salts of the foregoing;

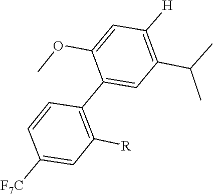

R

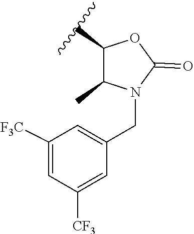 [SMILES: *[C@H]1OC(=O)N(Cc2cc(C(F)(F)F)cc(C(F)(F)F)c2)[C@H]1C]

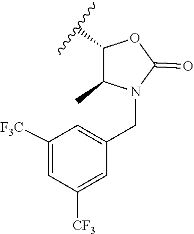 [SMILES: *[C@@H]1OC(=O)N(Cc2cc(C(F)(F)F)cc(C(F)(F)F)c2)[C@H]1C]

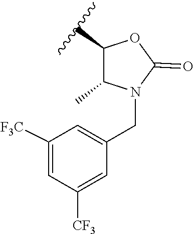 [SMILES: *[C@H]1OC(=O)N(Cc2cc(C(F)(F)F)cc(C(F)(F)F)c2)[C@@H]1C]

and pharmaceutically acceptable salts of the foregoing;

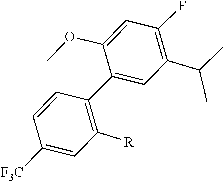

R

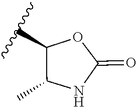

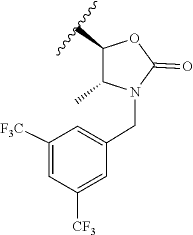 [SMILES: *[C@H]1OC(=O)N(Cc2cc(C(F)(F)F)cc(C(F)(F)F)c2)[C@@H]1C]

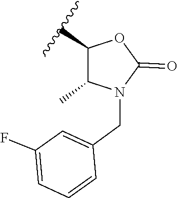 [SMILES: *[C@H]1OC(=O)N(Cc2cccc(F)c2)[C@@H]1C]

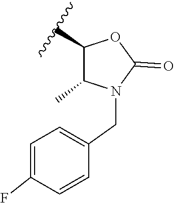 [SMILES: *[C@H]1OC(=O)N(Cc2ccc(F)cc2)[C@@H]1C]

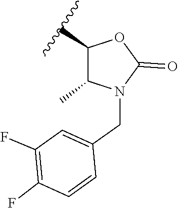 [SMILES: *[C@H]1OC(=O)N(Cc2ccc(F)c(F)c2)[C@@H]1C]

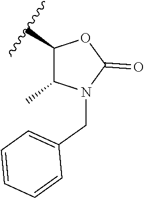 [SMILES: *[C@H]1OC(=O)N(Cc2ccccc2)[C@@H]1C]

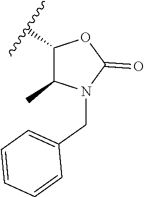 [SMILES: *[C@@H]1OC(=O)N(Cc2ccccc2)[C@H]1C]

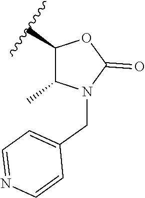 [SMILES: *[C@H]1OC(=O)N(Cc2ccncc2)[C@@H]1C]

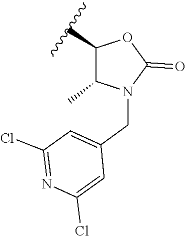 [SMILES: *[C@H]1OC(=O)N(Cc2cc(Cl)nc(Cl)c2)[C@@H]1C]

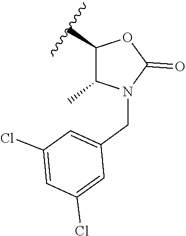 [SMILES: *[C@H]1OC(=O)N(Cc2cc(Cl)cc(Cl)c2)[C@@H]1C]

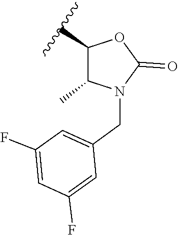 [SMILES: *[C@H]1OC(=O)N(Cc2cc(F)cc(F)c2)[C@@H]1C]

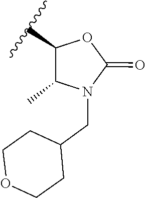 [SMILES: *[C@H]1OC(=O)N(CC2CCOCC2)[C@@H]1C]

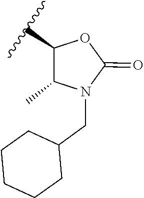 [SMILES: *[C@H]1OC(=O)N(CC2CCCCC2)[C@@H]1C]

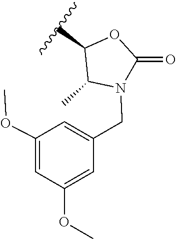 [SMILES: *[C@H]1OC(=O)N(Cc2cc(OC)cc(OC)c2)[C@@H]1C]

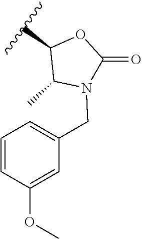 [SMILES: *[C@H]1OC(=O)N(Cc2cccc(OC)c2)[C@@H]1C]

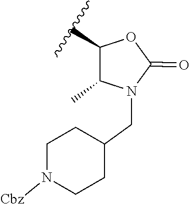 [SMILES: *[C@H]1OC(=O)N(CC2CCN(C(=O)OCc3ccccc3)CC2)[C@@H]1C]

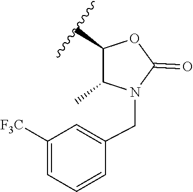 [SMILES: *[C@H]1OC(=O)N(Cc2cccc(C(F)(F)F)c2)[C@@H]1C]

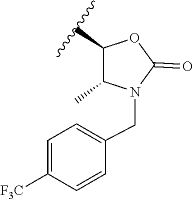 [SMILES: *[C@H]1OC(=O)N(Cc2ccc(C(F)(F)F)cc2)[C@@H]1C]

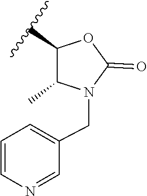 [SMILES: *[C@H]1OC(=O)N(Cc2cccnc2)[C@@H]1C]

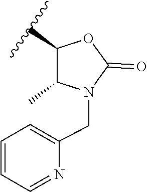 [SMILES: *[C@H]1OC(=O)N(Cc2ccccn2)[C@@H]1C]

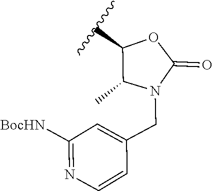 [SMILES: *[C@H]1OC(=O)N(Cc2ccnc(NC(=O)OC(C)(C)C)c2)[C@@H]1C]

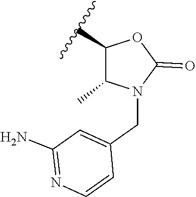 [SMILES: *[C@H]1OC(=O)N(Cc2ccnc(N)c2)[C@@H]1C]

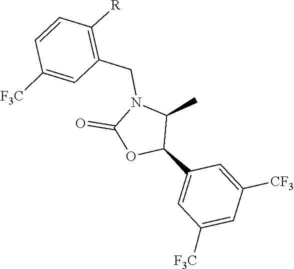

R

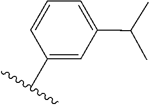 [SMILES: *c1cccc(C(C)C)c1]

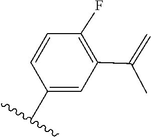 [SMILES: *c1ccc(F)c(C(=C)C)c1]

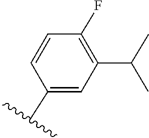 [SMILES: *c1ccc(F)c(C(C)C)c1]

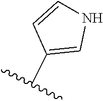 [SMILES: *c1cc[nH]c1]

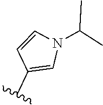 [SMILES: *c1ccn(C(C)C)c1]

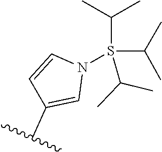 [SMILES: *c1ccn(S(C(C)C)(C(C)C)C(C)C)c1]

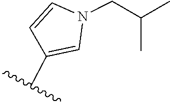 [SMILES: *c1ccn(CC(C)C)c1]

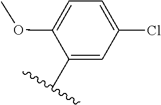 [SMILES: *c1cc(Cl)ccc1OC]

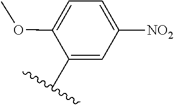 [SMILES: *c1cc([N+](=O)[O-])ccc1OC]

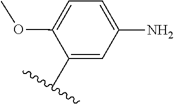 [SMILES: *c1cc(N)ccc1OC]

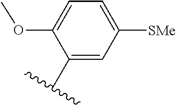 [SMILES: *c1cc(SC)ccc1OC]

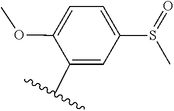 [SMILES: *c1cc(S(C)=O)ccc1OC]

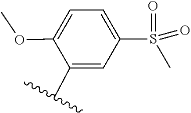 [SMILES: *c1cc(S(C)(=O)=O)ccc1OC]

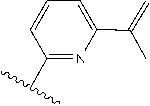 [SMILES: *c1cccc(C(=C)C)n1]

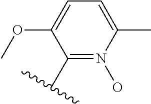

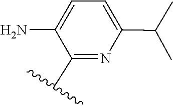 [SMILES: *c1nc(C(C)C)ccc1N]

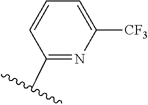 [SMILES: *c1cccc(C(F)(F)F)n1]

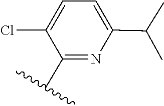 [SMILES: *c1nc(C(C)C)ccc1Cl]

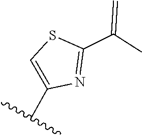 [SMILES: *c1csc(C(=C)C)n1]

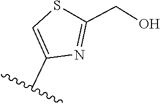 [SMILES: *c1csc(CO)n1]

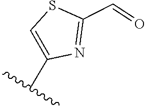 [SMILES: *c1csc(C=O)n1]

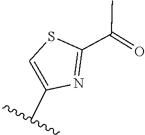 [SMILES: *c1csc(C(C)=O)n1]

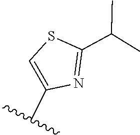 [SMILES: *c1csc(C(C)C)n1]

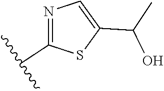 [SMILES: *c1ncc(C(C)O)s1]

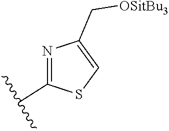

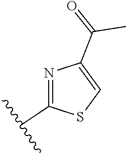 [SMILES: *c1nc(C(C)=O)cs1]

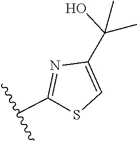 [SMILES: *c1nc(C(C)(C)O)cs1]

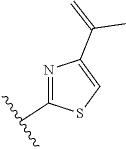 [SMILES: *c1nc(C(=C)C)cs1]

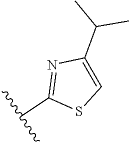 [SMILES: *c1nc(C(C)C)cs1]

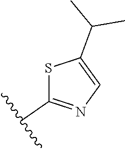 [SMILES: *c1ncc(C(C)C)s1]

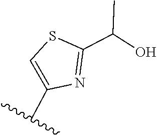 [SMILES: *c1csc(C(C)O)n1]

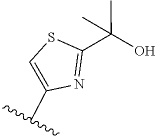 [SMILES: *c1csc(C(C)(C)O)n1]

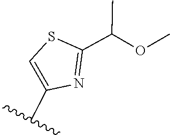 [SMILES: *c1csc(C(C)OC)n1]

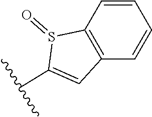 [SMILES: *C1=Cc2ccccc2S1=O]

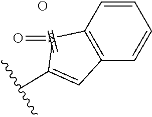

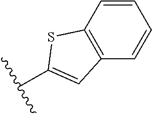 [SMILES: *c1cc2ccccc2s1]

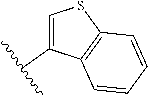 [SMILES: *c1csc2ccccc12]

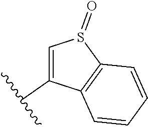 [SMILES: *C1=CS(=O)c2ccccc21]

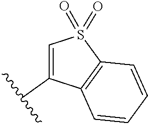 [SMILES: *C1=CS(=O)(=O)c2ccccc21]

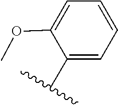 [SMILES: *c1ccccc1OC]

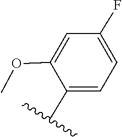 [SMILES: *c1ccc(F)cc1OC]

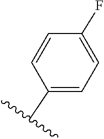 [SMILES: *c1ccc(F)cc1]

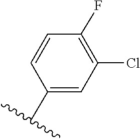 [SMILES: *c1ccc(F)c(Cl)c1]

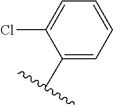 [SMILES: *c1ccccc1Cl]

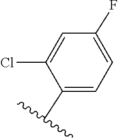 [SMILES: *c1ccc(F)cc1Cl]

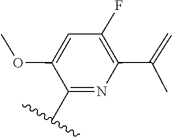 [SMILES: *c1nc(C(=C)C)c(F)cc1OC]

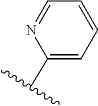 [SMILES: *c1ccccn1]

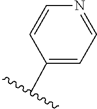 [SMILES: *c1ccncc1]

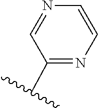 [SMILES: *c1cnccn1]

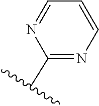 [SMILES: *c1ncccn1]

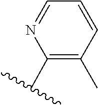 [SMILES: *c1ncccc1C]

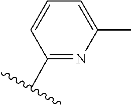 [SMILES: *c1cccc(C)n1]

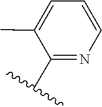 [SMILES: *c1ncccc1C]

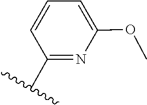 [SMILES: *c1cccc(OC)n1]

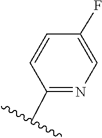 [SMILES: *c1ccc(F)cn1]

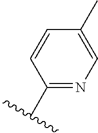 [SMILES: *c1ccc(C)cn1]

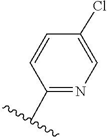 [SMILES: *c1ccc(Cl)cn1]

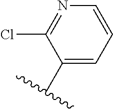 [SMILES: *c1cccnc1Cl]

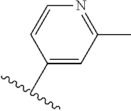 [SMILES: *c1ccnc(C)c1]

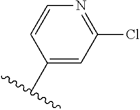 [SMILES: *c1ccnc(Cl)c1]

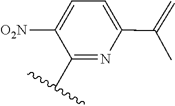 [SMILES: *c1nc(C(=C)C)ccc1[N+](=O)[O-]]

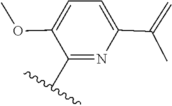 [SMILES: *c1nc(C(=C)C)ccc1OC]

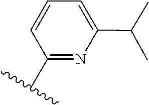 [SMILES: *c1cccc(C(C)C)n1]

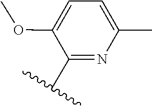

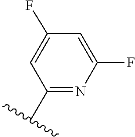 [SMILES: *c1cc(F)cc(F)n1]

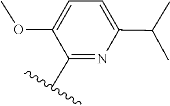 [SMILES: *c1nc(C(C)C)ccc1OC]

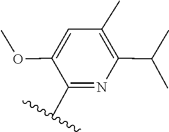 [SMILES: *c1nc(C(C)C)c(C)cc1OC]

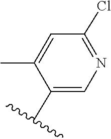 [SMILES: *c1cnc(Cl)cc1C]

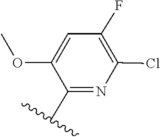 [SMILES: *c1nc(Cl)c(F)cc1OC]

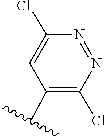 [SMILES: *c1cc(Cl)nnc1Cl]

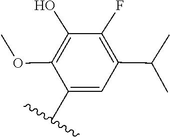 [SMILES: *c1cc(C(C)C)c(F)c(O)c1OC]

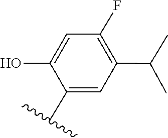 [SMILES: *c1cc(C(C)C)c(F)cc1O]

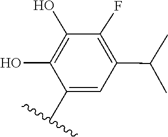 [SMILES: *c1cc(C(C)C)c(F)c(O)c1O]

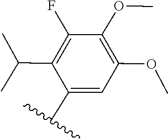 [SMILES: *c1cc(OC)c(OC)c(F)c1C(C)C]

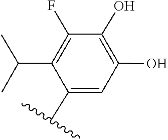 [SMILES: *c1cc(O)c(O)c(F)c1C(C)C]

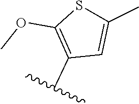

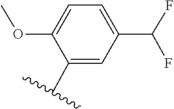 [SMILES: *c1cc(C(F)F)ccc1OC]

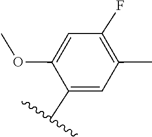

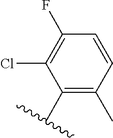 [SMILES: *c1c(C)ccc(F)c1Cl]

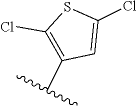 [SMILES: *c1cc(Cl)sc1Cl]

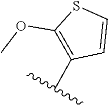 [SMILES: *c1ccsc1OC]

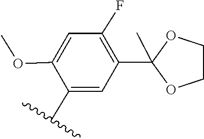 [SMILES: *c1cc(C2(C)OCCO2)c(F)cc1OC]

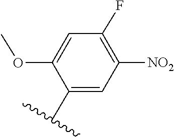 [SMILES: *c1cc([N+](=O)[O-])c(F)cc1OC]

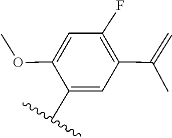 [SMILES: *c1cc(C(=C)C)c(F)cc1OC]

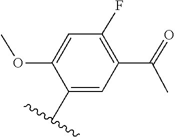 [SMILES: *c1cc(C(C)=O)c(F)cc1OC]

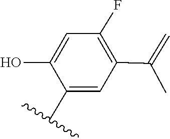 [SMILES: *c1cc(C(=C)C)c(F)cc1O]

and pharmaceutically acceptable salts of the foregoing.

Additional CETP inhibitors useful in the compositions and methods of the invention are disclosed in US20150374675 A1 and include, but are not limited to:
S-[2-(1-isopentylcyclohexanecarbonylamino)phenyl]2,2-dimethylthiopropionate;
S-[2-(1-isopentylcyclohexanecarbonylamino)phenyl]2-acetylamino-3-phenylthiopropionate;
S-[2-(1-isopentylcyclohexanecarbonylamino)phenyl]3-pyridinethiocarboxylate;
S-[2-(1-isopentylcyclohexanecarbonylamino)phenyl]chlorothioacetate;
S-[2-(1-isopentylcyclohexanecarbonylamino)phenyl]methoxythioacetate;
S-[2-(1-isopentylcyclohexanecarbonylamino)phenyl]thiopropionate;
S-[2-(1-isopentylcyclohexanecarbonylamino)phenyl]phenoxy-thioacetate;
S-[2-(1-isopentylcyclohexanecarbonylamino)phenyl]2-methylthiopropionate;
S-[2-(1-isopentylcyclohexanecarbonylamino)phenyl]4-chlorophenoxythioacetate;
S-[2-(1-isopentylcyclohexanecarbonylamino)phenyl]cyclopropanethiocarboxylate;
S-[2-(1-isopentylcyclohexanecarbonylamino)phenyl]2-acetylamino-4-carbamoylthiobutyrate;
S-[2-(1-isopentylcyclohexanecarbonylamino)phenyl]2-hydroxy-2-methylthiopropionate;
S-[2-(1-isopentylcyclopentanecarbonylamino)phenyl]2,2-dimethylthiopropionate;
S-[2-(1-isopentylcyclopentanecarbonylamino)phenyl]thioacetate;
S-[4,5-dichloro-2-(1-isopentylcyclohexanecarbonylamino)-phenyl]2,2-dimethylthiopropionate;
S-[4,5-dichloro-2-(1-isopentylcyclopentanecarbonylamino)-phenyl]2,2-dimethylthiopropionate;
S-[2-(1-isopentylcyclohexanecarbonylamino)-4-trifluoromethylphenyl]2,2-dimethylthiopropionate;
O-methyl S-[2-(1-isopentylcyclohexanecarbonylamino phenyl monothiocarbonate;
S-[2-(1-methylcyclohexanecarbonylamino)phenyl]S-phenyldithiocarbonate;
S-[2-(1-isopentylcyclohexanecarbonylamino)phenyl]N-phenylthiocarbamate;
S-[2-(pivaloylamino)-4-trifluoromethylphenyl]2,2-dimethylthiopropionate;
S-[4,5-dichloro-2-(1-cyclopropylcyclohexanecarbonylamino)phenyl]2,2-dimethylthiopropionate;
S-[4,5-dichloro-2-(2-cyclohexylpropionylamino)phenyl]2,2-dimethylthiopropionate;
S-[4,5-dichloro-2-(1-pentylcyclohexanecarbonylamino)-phenyl]2,2-dimethylthiopropionate;
S-[4,5-dichloro-2-(1-cyclopropylmethylcyclohexanecarbonylamino)phenyl]2,2-dimethylthiopropionate;
S-[4,5-dichloro-2-(1-cyclohexylmethylcyclohexanecarbonylamino)phenyl]2,2-dimethylthiopropionate;
S-[4,5-dichloro-2-(1-isopropylcyclohexanecarbonylamino)-phenyl]2,2-dimethylthiopropionate;
S-[4,5-dichloro-2-(1-isopentylcycloheptanecarbonylamino)-phenyl]2,2-dimethylthiopropionate;
S-[4,5-dichloro-2-(1-isopentylcyclobutanecarbonylamino)-phenyl]2,2-dimethylthiopropionate;
S-[2-(1-isopentylcyclohexanecarbonylamino)-4-nitrophenyl]2,2-dimethylthiopropionate;
S-[4-cyano-2-(1-isopentylcyclohexanecarbonylamino)phenyl]2,2-dimethylthiopropionate;
S-[4-chloro-2-(1-isopentylcyclohexanecarbonylamino)phenyl]2,2-dimethylthiopropionate;
S-[5-chloro-2-(1-isopentylcyclohexanecarbonylamino)phenyl]2,2-dimethylthiopropionate;
S-[4-fluoro-2-(1-isopentylcyclohexanecarbonylamino)phenyl]2,2-dimethylthiopropionate;
S-[4,5-difluoro-2-(1-isopentylcyclohexanecarbonylamino)-phenyl]2,2-dimethylthiopropionate;
S-[5-fluoro-2-(1-isopentylcyclohexanecarbonylamino)phenyl]2,2-dimethylthiopropionate; bis-[4,5-dichloro-2-(1-isopentylcyclohexanecarbonylamino)-phenyl]disulfide;
2-tetrahydrofurylmethyl 2-(1-isopentylcyclohexanecarbonylamino)phenyl disulfide;
N-(2-mercaptophenyl)-1-ethylcyclohexanecarboxamide;
N-(2-mercaptophenyl)-1-propylcyclohexanecarboxamide;
N-(2-mercaptophenyl)-1-butylcyclohexanecarboxamide;
N-(2-mercaptophenyl)-1-isobutylcyclohexanecarboxamide;
S-[2-(1-isopentylcyclohexanecarbonylamino)phenyl]cyclohexanethiocarboxylate;
S-[2-(1-isopentylcyclohexanecarbonylamino)phenyl]thiobenzoate;
S-[2-(1-isopentylcyclohexanecarbonylamino)phenyl]5-carboxythiopentanoate;
S-[2-(1-isopentylcyclohexanecarbonylamino)-4-methylphenyl]thioacetate; bis-[2-[1-(2-ethylbutyl)cyclohexanecarbonylamino]phenyl]disulfide;
N-(2-mercaptophenyl)-1-(2-ethylbutyl)cyclohexanecarboxamide;
S-[2-[1-(2-ethylbutyl)cyclohexanecarbonylamino]phenyl]2-methylthiopropionate;
S-[2-(1-isobutylcyclohexanecarbonylamino)phenyl]2-methylthiopropionate;
S-[2-[1-(2-ethylbutyl)cyclohexanecarbonylamino]phenyl]1-acetylpiperidine-4-thiocarboxylate;
S-[2-[1-(2-ethylbutyl)cyclohexanecarbonylamino]phenyl]thioacetate;
S-[2-[1-(2-ethylbutyl)cyclohexanecarbonylamino]phenyl]2,2-dimethylthiopropionate;
S-[2-[1-(2-ethylbutyl)cyclohexanecarbonylamino]phenyl]methoxythioacetate;
S-[2-[1-(2-ethylbutyl)cyclohexanecarbonylamino]phenyl]2-hydroxy-2-methylthiopropionate;
S-[2-[1-(2-ethylbutyl)cyclohexanecarbonylamino]phenyl]4-chlorophenoxythioacetate;
S-[2-(1-isobutylcyclohexanecarbonylamino)phenyl]4-chlorophenoxythioacetate; and
S-[2-(1-isobutylcyclohexanecarbonylamino)phenyl]-1-acetyl-piperidine-4-thiocarboxylate; and
   pharmaceutically acceptable salts of the foregoing.

Additional examples of CETP inhibitors useful in the compositions and methods of the invention include, but are not limited to: torcetrapib; dalcetrapib; anacetrapib; evacetrapib; obicetrapib; BMS-79531; CP-800,569; DRL-17822; JNJ-28545595; JNJ-28614872; BAY 19-4789; BAY 38-1315; 1,1,1-trifluoro-3-((3-phenoxyphenyl)(3-(1,1,2,2-tetrafluoroethoxy)benzyl)amino)propan-2-ol; (R)-3-((4-(4-chloro-3-ethylphenoxy)pyrimidin-2-yl)(3-(1,1,2,2-tetrafluoroethoxy)benzyl)amino)-1,1,1-trifluoropropan-2-ol; (R)-3-((3-(4-chloro-3-ethylphenoxy)phenyl)(3-(1,1,2,2-tetrafluoroethoxy)benzyl)amino)-1,1,1-trifluoropropan-2-ol (CP-800,569); N-(4-(5,7-dimethylbenzo[d]oxazol-2-yl)phenyl)-2-(o-tolyloxy)acetamide; 2-(4-chloro-2,3-dimethylphenoxy)-N-(4-(5-cyanobenzo[d]oxazol-2-yl)phenyl)acetamide; N-(4-(5-chlorobenzo[d]oxazol-2-yl)phenyl)-2-(o-tolyloxy)acetamide; N-(4-(5-chlorobenzo[d]oxazol-2-yl)phenyl)-2-(o-tolyloxy)acetamide; N-(4-(5-cyano-7-methylbenzo[d]oxazol-2-yl)phenyl)-2-(o-tolyloxy)acetamide; N-(4-(5-cyano-7-(2-hydroxypropan-2-yl)benzo[d]oxazol-2-yl)phenyl)-2-(o-tolyloxy)acetamide; 2-(4-((2-(3,3,3-trifluoro-2-methyl-2-(trifluoromethyl)propoxy)ethyl)amino)phenyl)benzo[d]oxazole-5-carbonitrile; tert-butyl 4-(2-((4-(5-cyanobenzo[d]oxazol-2-yl)phenyl)amino)-2-oxoethoxy)piperidine-1-carboxylate; N-(4-(5-cyano-7-methylbenzo[d]oxazol-2-yl)phenyl)-2-(4-(3-(trifluoromethyl)phenyl)piperazin-1-yl)acetamide; N-(4-(5-cyano-7-methylbenzo[d]oxazol-2-yl)phenyl)-2-(4-(4-(trifluoromethyl)phenyl)piperazin-1-yl)acetamide; N-(4-(5-cyano-7-methylbenzo[d]oxazol-2-yl)phenyl)-2-(4-(5-(trifluoromethyl)pyridin-2-yl)piperazin-1-yl)acetamide; 4-(5-cyano-7-methylbenzo[d]oxazol-2-yl)-N-((1-(4-(trifluoromethyl)phenyl)piperidin-4-yl)methyl)benzamide; 4-(5-cyano-7-isopropylbenzo[d]oxazol-2-yl)-N-((1-(5-(trifluoromethyl)pyridin-2-yl)piperidin-4-yl)methyl)benzamide; 4-(5-cyano-7-isopropylbenzo[d]oxazol-2-yl)-N-((1-(5-phenylpyridin-2-yl)piperidin-4-yl)methyl)benzamide; 4-(5-cyano-7-isopropylbenzo[d]oxazol-2-yl)-N-((1-(5-(2-isopropyl-5-methylphenyl)pyridin-2-yl)piperidin-4-yl)methyl)benzamide; 4-(5-cyano-7-isopropylbenzo[d]oxazol-2-yl)-N-((1-(5-(5-fluoro-2-isopropylphenyl)pyridin-2-yl)piperidin-4-yl)methyl)benzamide; (R)-4-(5-cyano-7-isopropylbenzo[d]oxazol-2-yl)-N-((2-oxo-3-(5-(2-(trifluoromethoxy)phenyl)pyridin-2-yl)oxazolidin-5-yl)methyl)benzamide; (S)-4-(5-cyano-7-isopropylbenzo[d]oxazol-2-yl)-N-((2-oxo-3-(5-(2-(trifluoromethoxy)phenyl)pyridin-2-yl)oxazolidin-5-yl)methyl)benzamide; (R)-4-(5-cyano-7-isopropylbenzo[d]oxazol-2-yl)-N-((5-methyl-2-oxo-3-(5-(2-(trifluoromethoxy)phenyl)pyridin-2-yl)oxazolidin-5-yl)methyl)benzamide; (S))-4-(5-cyano-7-isopropylbenzo[d]oxazol-2-yl)-N-((5-methyl-2-oxo-3-(5-(2-(trifluoromethoxy)phenyl)pyridin-2-yl)oxazolidin-5-yl)methyl)benzamide; N-((4-(4-(tert-butyl)phenyl)cyclohexyl)methyl)-4-(5-cyano-7-isopropylbenzo[d]oxazol-2-yl)benzamide; methyl (3,5-bis(trifluoromethyl)benzyl)((5′-isopropyl-2′-methoxy-4-(trifluoromethyl)-[1,1′-biphenyl]-2-yl)methyl)carbamate; methyl (3,5-bis(trifluoromethyl)benzyl)(2-((ethoxycarbonyl)(propyl)amino)-5-(trifluoromethyl)benzyl)carbamate; methyl (3,5-bis(trifluoromethyl)benzyl)(2-(2-oxooxazolidin-3-yl)-5-(trifluoromethyl)benzyl)carbamate; methyl (3,5-bis(trifluoromethyl)benzyl)(2-(2-oxoimidazolidin-1-yl)-5-(trifluoromethyl)benzyl)carbamate; 4-(3,5-bis(trifluoromethyl)phenyl)-3-((5′-isopropyl-2′-methoxy-4-(trifluoromethyl)-[1,1′-biphenyl]-2-yl)methyl)oxazolidin-2-one; (R)-4-(3,5-bis(trifluoromethyl)phenyl)-3-((5′-isopropyl-2′-methoxy-4-(trifluoromethyl)-[1,1′-biphenyl]-2-yl)methyl)oxazolidin-2-one; (S)-4-(3,5-bis(trifluoromethyl)phenyl)-3-((5′-isopropyl-2′-methoxy-4-(trifluoromethyl)-[1,1′-biphenyl]-2-yl)methyl)oxazolidin-2-one; (4R,5S)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; (4S,5R)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; (4R,5R)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; (4S,5S)-5-(3,5-bis(trifluoromethyl)phenyl)-3-((5′-isopropyl-2′-methoxy-4-(trifluoromethyl)-[1,1′-biphenyl]-2-yl)methyl)-4-methyloxazolidin-2-one; 5-(2,6-bis(trifluoromethyl)pyridin-4-yl)-3-((4′-fluoro-5′-isopropyl-2′-methoxy-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-2′-hydroxy-5′-isopropyl-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-2′,3′-dihydroxy-5′-isopropyl-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-2′,3′-dihydroxy-5′-(2-hydroxypropan-2-yl)-4-(trifluoromethyl)-[1,1′-biphenyl]-2-yl)methyl)-4-methyloxazolidin-2-one; (4S,5S)-5-(3,5-bis(trifluoromethyl)phenyl)-3-((4′-fluoro-5′-isopropyl-2′-methoxy-4-(trifluoromethyl)-3,4,5,6-tetrahydro-[1,1′-biphenyl]-2-yl)methyl)-4-methyloxazolidin-2-one; N-(6′-(((4S,5S)-5-(3,5-bis(trifluoromethyl)phenyl)-4-methyl-2-oxooxazolidin-3-yl)methyl)-2-methoxy-4′,4′-dimethyl-2′,3′,4′,5′-tetrahydro-[1,1′-biphenyl]-4-yl)-N-methylacetamide; (S)-5-(3,5-bis(trifluoromethyl)phenyl)-3-((4′-fluoro-5′-isopropyl-2′-methoxy-4-(trifluoromethyl)-[1,1′-biphenyl]-2-yl)methyl)-4,4-dimethyloxazolidin-2-one; 3-(6′-(((4S,5S)-5-(3,5-bis(trifluoromethyl)phenyl)-4-methyl-2-oxooxazolidin-3-yl)methyl)-2-methoxy-4′,4′-dimethyl-2′,3′,4′,5′-tetrahydro-[1,1′-biphenyl]-4-yl)-2,2-dimethylpropanoic acid; 3-(3-(2-(((4S,5S)-5-(3,5-bis(trifluoromethyl)phenyl)-4-methyl-2-oxooxazolidin-3-yl)methyl)-6-methoxypyridin-3-yl)-4-methoxyphenyl)propanoic acid; 3′-(6-(azetidin-1-yl)-2-(((4S,5S)-5-(3,5-bis(trifluoromethyl)phenyl)-4-methyl-2-oxooxazolidin-3-yl)methyl)pyridin-3-yl)-5′-fluoro-4′-methoxy-2-methyl-[1,1′-biphenyl]-4-carboxylic acid; isopropyl (2R,4S)-4-((3,5-bis(trifluoromethyl)benzyl)(2H-tetrazol-5-yl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-methyl-2H-tetrazol-5-yl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-(2-cyanoethyl)-2H-tetrazol-5-yl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-(2-hydroxyethyl)-2H-tetrazol-5-yl)amino)-2-ethyl-6-(trifluoromethyl)-3,4-dihydroquinoline-1(2H)-carboxylate; isopropyl (2R,4S)-4-((2-(2-aminoethyl)-2H-tetrazol-5-yl)(3,5-bis(trifluoromethyl)benzyl)amino)-2-ethyl-6-(trifluoromethyl)-3,4-dihydroquinoline-1(2H)-carboxylate; isopropyl (2R,4S)-4-((3,5-bis(trifluoromethyl)benzyl)(2-(2-hydroxypropyl)-2H-tetrazol-5-yl)amino)-2-ethyl-6-(trifluoromethyl)-3,4-dihydroquinoline-1(2H)-carboxylate; ethyl (2R,4S)-4-((3,5-bis(trifluoromethyl)benzyl)(2-methyl-2H-tetrazol-5-yl)amino)-2-ethyl-6-(trifluoromethyl)-3,4-dihydroquinoline-1(2H)-carboxylate; ethyl (2R,4S)-4-((3,5-bis(trifluoromethyl)benzyl)(2-methyl-2H-tetrazol-5-yl)amino)-2-ethyl-8-methyl-6-(trifluoromethyl)-3,4-dihydroquinoline-1(2H)-carboxylate; ethyl (2R,4S)-4-(N-(3,5-bis(trifluoromethyl)benzyl)acetamido)-2-ethyl-6-(trifluoromethyl)-3,4-dihydro-1,5-naphthyridine-1(2H)-carboxylate; ethyl (2R,4S)-4-(N-(3,5-bis(trifluoromethyl)benzyl)acetamido)-2-ethyl-6-methoxy-3,4-dihydro-1,5-naphthyridine-1(2H)-carboxylate; ethyl (2R,4S)-4-(N-(3,5-bis(trifluoromethyl)benzyl)acetamido)-6-(dimethylamino)-2-ethyl-3,4-dihydro-1,5-naphthyridine-1(2H)-carboxylate; ethyl (2R,4S)-4-((3,5-bis(trifluoromethyl)benzyl)(2-methyl-2H-tetrazol-5-yl)amino)-2-ethyl-6-(trifluoromethyl)-3,4-dihydro-1,5-naphthyridine-1(2H)-carboxylate; ethyl (2R,4S)-4-((3,5-bis(trifluoromethyl)benzyl)(2-methyl-2H-tetrazol-5-yl)amino)-2-ethyl-6-methoxy-3,4-dihydro-1,5-naphthyridine-1(2H)-carboxylate; ethyl (2R,4S)-4-((3,5-bis(trifluoromethyl)benzyl)(2-methyl-2H-tetrazol-5-yl)amino)-6-(dimethylamino)-2-ethyl-3,4-dihydro-1,5-naphthyridine-1(2H)-carboxylate; isopropyl (2R,4S)-4-((3,5-bis(trifluoromethyl)benzyl)(2-methyl-2H-tetrazol-5-yl)amino)-2-ethyl-6-(trifluoromethyl)-3,4-dihydro-1,5-naphthyridine-1(2H)-carboxylate; isopropyl (2R,4S)-4-((3-chloro-5-(trifluoromethyl)benzyl)(2-methyl-2H-tetrazol-5-yl)amino)-2-ethyl-6-(trifluoromethyl)-3,4-dihydro-1,5-naphthyridine-1(2H)-carboxylate; isopropyl (2R,4S)-4-((3,5-dichlorobenzyl)(2-methyl-2H-tetrazol-5-yl)amino)-2-ethyl-6-methyl-3,4-dihydro-1,5-naphthyridine-1(2H)-carboxylate; 5-(((3,5-bis(trifluoromethyl)benzyl)(2-methyl-2H-tetrazol-5-yl)amino)methyl)-N-(cyclopentylmethyl)-N-ethyl-1,3-dimethyl-1H-pyrazolo[3,4-b]pyridin-6-amine; 6-(((2-(bis(cyclopropylmethyl)amino)-7,7-dimethyl-6,7-dihydro-5H-cyclopenta[b]pyridin-3-yl)methyl)(3,5-bis(trifluoromethyl)benzyl)amino)benzo[d]oxazol-2(3H)-one; 3-(((3,5-bis(trifluoromethyl)benzyl)(5-morpholinopyrimidin-2-yl)amino)methyl)-N,N-bis(cyclopropylmethyl)-7,7-dimethyl-6,7-dihydro-5H-cyclopenta[b]pyridin-2-amine; isopropyl (2R)-4-((3,5-bis(trifluoromethyl)benzyl)(5-(1-methyl-1H-pyrazol-4-yl)pyrimidin-2-yl)amino)-2-ethylpyrrolidine-1-carboxylate; 3-(((3,5-bis(trifluoromethyl)benzyl)(2-methyl-2H-tetrazol-5-yl)amino)methyl)-5-bromo-N-(cyclopentylmethyl)-N-ethyl-6-methylpyridin-2-amine; 3-(((3,5-bis(trifluoromethyl)benzyl)(2-methyl-2H-tetrazol-5-yl)amino)methyl)-N-(cyclopentylmethyl)-N-ethyl-6-methyl-5-(methylthio)pyridin-2-amine; ((2R)-4-((3,5-bis(trifluoromethyl)benzyl)(5-(1-methyl-1H-pyrazol-4-yl)pyrimidin-2-yl)amino)-2-ethylpyrrolidin-1-yl)(cyclohexyl)methanone; (1r,4r)-4-(((2-(((3,5-bis(trifluoromethyl)benzyl)(2-methyl-2H-tetrazol-5-yl)amino)methyl)-4-(trifluoromethyl)phenyl)(ethyl)amino)methyl)cyclohexane-1-carboxylic acid; 3-((((3-((cyclopentylmethyl)(ethyl)amino)-5,6,7,8-tetrahydronaphthalen-2-yl)methyl)(2-methyl-2H-tetrazol-5-yl)amino)methyl)-5-(trifluoromethyl)benzonitrile; (1R,4r)-4-(((2R,6S)-4-((3,5-bis(trifluoromethyl)benzyl)(5-(1-methyl-1H-pyrazol-4-yl)pyrimidin-2-yl)amino)-2,6-diethylpiperidine-1-carbonyl)oxy)cyclohexane-1-carboxylic acid; (1R,3R)-3-(((2R,6S)-4-((3,5-bis(trifluoromethyl)benzyl)(5-(1-methyl-1H-pyrazol-4-yl)pyrimidin-2-yl)amino)-2,6-diethylpiperidine-1-carbonyl)oxy)cyclobutane-1-carboxylic acid; 1-(2-((3,5-bis(trifluoromethyl)benzyl)(2-(ethyl(2-methoxyethyl)amino)benzyl)amino)pyrimidin-5-yl)piperidine-4-carboxylic acid; 5-(((1-(3,5-bis(trifluoromethyl)phenyl)ethyl)(5-(2-(methylsulfonyl)ethoxy)pyrimidin-2-yl)amino)methyl)-N-(cyclopentylmethyl)-N-ethyl-1,3-dimethyl-1H-indazol-6-amine; N-(1-(3,5-bis(trifluoromethyl)phenyl)ethyl)-N-(2-((cyclopentylmethyl)(ethyl)amino)-5-(trifluoromethyl)benzyl)-5-(2-(methylsulfonyl)ethoxy)pyrimidin-2-amine; 4-((2-((3,5-bis(trifluoromethyl)benzyl)((3-((cyclopropylmethyl)(propyl)amino)quinolin-2-yl)methyl)amino)pyrimidin-5-yl)oxy)butanoic acid; 3-((((3-((cyclopentylmethyl)(ethyl)amino)-6-methoxypyridin-2-yl)methyl)(5-(2-(methylsulfonyl)ethoxy)pyrimidin-2-yl)amino)methyl)-5-(trifluoromethyl)benzonitrile; 2-((1S,4r)-4-(((2-((((S)-1-(3,5-bis(trifluoromethyl)phenyl)ethyl)(5-(2-(methylsulfonyl)ethoxy)pyrimidin-2-yl)amino)methyl)-4-(trifluoromethyl)phenyl)(ethyl)amino)methyl)cyclohexyl)acetic acid; ethyl (2R,4S)-4-((3,5-bis(trifluoromethyl)benzyl)(5-(2-(methylsulfonyl)ethoxy)pyrimidin-2-yl)amino)-2-ethyl-6-methoxy-3,4-dihydro-1,5-naphthyridine-1(2H)-carboxylate; ethyl (2R,4S)-4-((3,5-bis(trifluoromethyl)benzyl)(5-morpholinopyrimidin-2-yl)amino)-2-ethyl-6-(trifluoromethyl)-3,4-dihydroquinoline-1(2H)-carboxylate; ethyl (2R,4S)-4-((3,5-bis(trifluoromethyl)benzyl)(5-morpholinopyrimidin-2-yl)amino)-2-ethyl-6-methoxy-3,4-dihydro-1,5-naphthyridine-1(2H)-carboxylate; isopropyl 5-((3,5-bis(trifluoromethyl)benzyl)(2-methyl-2H-tetrazol-5-yl)amino)-7-methyl-8-(trifluoromethyl)-2,3,4,5-tetrahydro-1H-benzo[b]azepine-1-carboxylate; isopropyl 5-(N-(3,5-bis(trifluoromethyl)benzyl)acetamido)-7-methyl-2,3,4,5-tetrahydro-1H-benzo[b]azepine-1-carboxylate; 3-(5-(4-chloro-3-ethylphenoxy)-2-(3-(1,1,2,2-tetrafluoroethoxy)phenyl)-3,4-dihydroquinolin-1(2H)-yl)-1,1,1-trifluoropropan-2-ol; (S)-1,1,1-trifluoro-3-((R)-2-(3-(1,1,2,2-tetrafluoroethoxy)phenyl)-5-(4-(trifluoromethoxy)phenyl)-3,4-dihydroquinolin-1(2H)-yl)propan-2-ol (JNJ-28545595); (S)-1,1,1-trifluoro-3-((S)-3-(3-(1,1,2,2-tetrafluoroethoxy)phenyl)-8-(4-(trifluoromethoxy)phenyl)-2,3-dihydro-4H-benzo[b][1,4]oxazin-4-yl)propan-2-ol (JNJ-28614872); (R)-3-((R)-4-(3-(difluoromethoxy)benzyl)-2-(3-(trifluoromethyl)phenyl)-3,4-dihydroquinoxalin-1(2H)-yl)-1,1,1-trifluoropropan-2-ol; (S)-(2-cyclopentyl-4-ethyl-5-hydroxy-7,7-dimethyl-5,6,7,8-tetrahydroquinolin-3-yl)(4-(trifluoromethyl)phenyl)methanone; (S)-2-cyclopentyl-3-((S)-fluoro(4-(trifluoromethyl)phenyl)methyl)-4-(4-fluorophenyl)-7,7-dimethyl-5,6,7,8-tetrahydroquinolin-5-ol (BAY 19-4789); (S)-3′-((S)-fluoro(4-(trifluoromethyl)phenyl)methyl)-4′-(4-fluorophenyl)-2′-isopropyl-5′,8′-dihydro-6′H-spiro[cyclobutane-1,7′-quinolin]-5′-01 (BAY 38-1315); (S)-4-cyclohexyl-2-cyclopentyl-3-((S)-hydroxy(4-(trifluoromethyl)phenyl)methyl)-7,7-dimethyl-5,6,7,8-tetrahydroquinolin-5-ol; (S)-4-cyclohexyl-2-cyclopentyl-3-((S)-fluoro(4-(trifluoromethyl)phenyl)methyl)-7,7-dimethyl-5,6,7,8-tetrahydroquinolin-5-ol; (S)-4-cyclohexyl-2-cyclopentyl-7,7-dimethyl-3-(4-(trifluoromethyl)benzyl)-5,6,7,8-tetrahydroquinolin-5-ol; (S)-6′-((S)-fluoro(4-(trifluoromethyl)phenyl)methyl)-5′-(4-fluorophenyl)-7′-isopropyl-3′,4′-dihydrospiro[cyclobutane-1,2′-pyrano[2,3-b]pyridin]-4′-ol; (S)-6′-((S)-fluoro(4-(trifluoromethyl)phenyl)methyl)-5′-(4-fluorophenyl)-7′-isopropyl-3′,4′-dihydrospiro[cyclopropane-1,2′-pyrano[2,3-b]pyridin]-4′-ol; (S)-5′-(4-fluorophenyl)-6′-((S)-hydroxy(4-(trifluoromethyl)phenyl)methyl)-7′-isopropyl-3′,4′-dihydrospiro[cyclobutane-1,2′-pyrano[2,3-b]pyridin]-4′-ol; (S)-5′-(4-fluorophenyl)-6′-((S)-hydroxy(4-(trifluoromethyl)phenyl)methyl)-7′-isopropyl-3′,4′-dihydrospiro[cyclopropane-1,2′-pyrano[2,3-b]pyridin]-4′-ol; (S)-(2-cyclopentyl-5-hydroxy-4-isopropyl-7,7-dimethyl-5,6,7,8-tetrahydroquinolin-3-yl)(4-(trifluoromethyl)phenyl)methanone; (S)-(2-cyclopentyl-5-hydroxy-7,7-dimethyl-4-(penta-1,3-diyn-1-yl)-5,6,7,8-tetrahydroquinolin-3-yl)(4-(trifluoromethyl)phenyl)methanone compound with dihydrogen (1:3); (S)-(2-cyclopentyl-4-(hexa-1,3,5-triyn-1-yl)-5-hydroxy-7,7-dimethyl-5,6,7,8-tetrahydroquinolin-3-yl)(4-(trifluoromethyl)phenyl)methanone compound with dihydrogen (1:5); (S)-(2′-cyclopentyl-5′-hydroxy-4′-isopropyl-5′,8′-dihydro-6′H-spiro[cyclobutane-1,7′-quinolin]-3′-yl)(4-(trifluoromethyl)phenyl)methanone; (S)-(2′-cyclopentyl-5′-hydroxy-4′-(penta-1,3-diyn-1-yl)-5′,8′-dihydro-6′H-spiro[cyclobutane-1,7′-quinolin]-3′-yl)(4-(trifluoromethyl)phenyl)methanone compound with dihydrogen (1:3); (S)-(2′-cyclopentyl-4′-(hexa-1,3,5-triyn-1-yl)-5′-hydroxy-5′,8′-dihydro-6′H-spiro[cyclobutane-1,7′-quinolin]-3′-yl)(4-(trifluoromethyl)phenyl)methanone compound with dihydrogen (1:5); (S)-(4-cyclohexyl-5-hydroxy-2-isopropyl-7,7-dimethyl-5,6,7,8-tetrahydroquinolin-3-yl)(4-(trifluoromethyl)phenyl)methanone; (S)-(4′-cyclohexyl-5′-hydroxy-2′-isopropyl-5′,8′-dihydro-6′H-spiro[cyclobutane-1,7′-quinolin]-3′-yl)(4-(trifluoromethyl)phenyl)methanone; (S)-4-(4,4-difluorocyclohexyl)-3-((S)-fluoro(4-(trifluoromethyl)phenyl)methyl)-2-(1-(5-(3-hydroxy-3-methylbutoxy)pyrimidin-2-yl)piperidin-4-yl)-7,7-dimethyl-5,6,7,8-tetrahydroquinolin-5-ol; N-((2-(4-((S)-4-(4,4-difluorocyclohexyl)-3-((S)-fluoro(4-(trifluoromethyl)phenyl)methyl)-5-hydroxy-7,7-dimethyl-5,6,7,8-tetrahydroquinolin-2-yl)piperidin-1-yl)pyrimidin-5-yl)methyl)-N-methylmethanesulfonamide; (S)-4-(4,4-difluorocyclohexyl)-3-((S)-fluoro(4-(trifluoromethyl)phenyl)methyl)-7,7-dimethyl-2-(1-(5-((1-methylpiperidin-4-yl)oxy)pyrimidin-2-yl)piperidin-4-yl)-5,6,7,8-tetrahydroquinolin-5-ol; (S)-6′-((R)-fluoro(4-(trifluoromethyl)phenyl)methyl)-5′-(4-fluorophenyl)-7′-isopropyl-3′,4′-dihydrospiro[cyclobutane-1,2′-pyrano[2,3-b]pyridin]-4′-ol; (S)-6′-((R)-fluoro(4-(trifluoromethyl)phenyl)methyl)-5′-(4-fluorophenyl)-7′-isopropyl-3′,4′-dihydrospiro[cyclopropane-1,2′-pyrano[2,3-b]pyridin]-4′-ol; 2-phenyl-1-(pyridin-2-yl)-1-(3-(trifluoromethyl)phenyl)ethyl 3,3-dimethylbutanoate; (S)-1-(1-(5-chloropyridin-2-yl)-1-(3-fluoro-5-(1,1,2,2-tetrafluoroethoxy)phenyl)-2-phenylethyl)-3-cyclopentylurea; (S)—N-(1-(5-chloropyridin-2-yl)-1-(3-fluoro-5-(1,1,2,2-tetrafluoroethoxy)phenyl)-2-phenylethyl)-4-fluoro-3-(trifluoromethyl)benzamide; 1-((S)-1-(5-chloropyridin-2-yl)-1-(3-fluoro-5-(1,1,2,2-tetrafluoroethoxy)phenyl)-2-phenylethyl)-3-((R)-3,3-difluorocyclopentyl)urea; (S)-1-(1-(5-chloropyridin-2-yl)-1-(3-fluoro-5-(1,1,2,2-tetrafluoroethoxy)phenyl)-2-phenylethyl)-3-(3,3-difluorocyclobutyl)urea; (3′R,9'S)-4′-isopropyl-7′,7′-dimethyl-3′-(4-(trifluoromethyl)phenyl)-6′,7′,8′,9′-tetrahydro-3′H-spiro[cyclopentane-1,1′-furo[3,4-c]quinolin]-9′-ol; (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol; (3′R,6′R,9'S)-4′-isopropyl-3′-(4-(trifluoromethyl)phenyl)-2″,3′,3″,5″,6′,6″,8′,9′-octahydrodispiro[cyclopropane-1,7′-furo[3,4-c]quinoline-1′,4″-pyran]-6′,9′-diol; (S)-1-(1-(5-chloropyridin-2-yl)-1-(3-fluoro-5-(1,1,2,2-tetrafluoroethoxy)phenyl)-2-phenylethyl)-3-(2,2,2-trifluoroethyl)urea; (R)-3-(((S)-3-(5-chloropyridin-2-yl)-3-(3-fluoro-5-(1,1,2,2-tetrafluoroethoxy)phenyl)-4-phenylbutyl)amino)-1,1,1-trifluoropropan-2-ol; (R)-3-(((R)-2-(5-chloropyridin-2-yl)-2-(3-fluoro-5-(1,1,2,2-tetrafluoroethoxy)phenyl)-3-phenylpropyl)amino)-1,1,1-trifluoropropan-2-ol; 5-chloro-6-fluoro-N-(3-(trifluoromethyl)phenethyl)-N-(4-(trimethylsilyl)benzyl)-1H-indole-7-carboxamide; 5-chloro-6-fluoro-N-(3-(trifluoromethoxy)phenethyl)-N-(4-(trimethylsilyl)benzyl)-1H-indole-7-carboxamide; Dacetrapib; N-(4-(tert-butyl)benzyl)-5-chloro-N-(3-(trifluoromethyl)phenethyl)-1H-pyrrolo[2,3-c]pyridine-7-carboxamide; 3,5-dichloro-N-(4-chlorophenethyl)-N-(4-(perfluoropropan-2-yl)benzyl)benzamide; and N-((5-(tert-butyl)thiophen-2-yl)methyl)-5-chloro-2-(methylamino)-N-(4-(trifluoromethyl)phenethyl)nicotinamide; and pharmaceutically acceptable salts of the foregoing.

In some embodiments, the CETP inhibitor is an antibody or peptide. U.S. Pat. No. 5,519,001, herein incorporated by reference, describes a 36 amino acid peptide derived from baboon apo C-1 that inhibits CETP activity. Cho et al. (Biochim. Biophys. Acta (1998) 1391: 133-144) describes a peptide from hog plasma that inhibits human CETP. Bonin et al. (J. Peptide Res. (1998) 51, 216-225) discloses a decapeptide inhibitor of CETP. A depspeptide fungal metabolite is disclosed as a CETP inhibitor by Hedge et al. in Bioorg. Med. Chem. Lett., (1998) 8:1277-80. An anti-CETP antibody has been described in WO2013075040 A1, herein incorporated by reference.

### ADCY Inhibitors

ADCY inhibitors that are useful in the compositions and methods of the invention include small molecules, anti-ADCY antibodies and peptides that inhibit or suppress adenylate cyclase expression or activity. In some embodiments, the ADCY inhibitor inhibits or suppresses adenylate cyclase expression or activity of one or more of ADCY, ADCY2, ADCY3, ADCY4, ADCY5, ADCY6, ADCY7, ADCY8, ADCY9 and ADCY10. In some embodiments, the ADCY inhibitor is an ADCY1, ADCY2, ADCY3, ADCY4, ADCY5, ADCY6, ADCY7, ADCY8, ADCY9, or ADCY10 inhibitor.

The following table lists illustrative ADCY inhibitors. These ADCY inhibitors and pharmaceutically acceptable salts thereof are useful in the methods and compositions of the present invention. Each compound's structure is depicted at the immediate right of its name.

| Compound | Structure | Compound | Structure |
|---|---|---|---|
| SQ 22,536 | 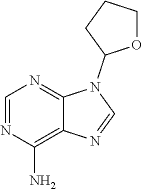 [SMILES: Nc1ncnc2c1ncn2C1CCCO1] | 2′,5′-dd-3′- ATP | 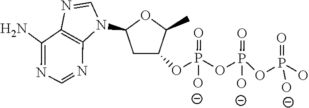 [SMILES: C[C@@H]1O[C@H](n2cnc3c(N)ncnc32)C[C@H]1OP(=O)([O-])OP(=O)([O-])OP(=O)([O-])O] |
| NKY80 | 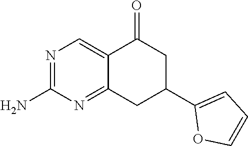 [SMILES: Nc1ncc2c(n1)CC(c1ccco1)CC2=O] | AraAde | 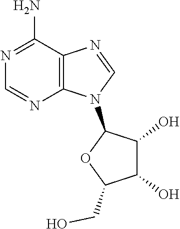 [SMILES: Nc1ncnc2c1ncn2[C@@H]1O[C@@H](CO)[C@@H](O)[C@H]1O] |
| vidarabine | 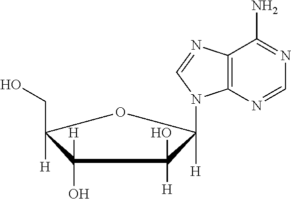 | PMC6 | 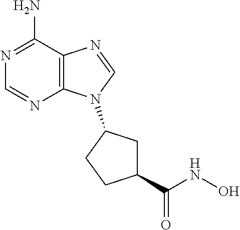 [SMILES: Nc1ncnc2c1ncn2[C@H]1CC[C@H](C(=O)NO)C1] |
| NB001 | 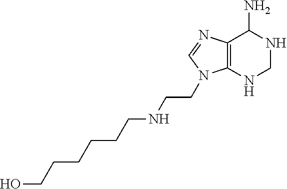 [SMILES: NC1NCNc2c1ncn2CCNCCCCCCO] | MDL 12330A | 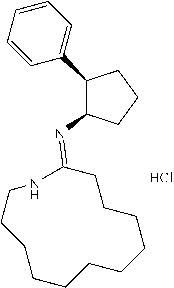 [SMILES: c1ccc([C@H]2CCC[C@H]2/N=C2\CCCCCCCCCCCN2)cc1 ; Cl] |
| BODIPY-FS | 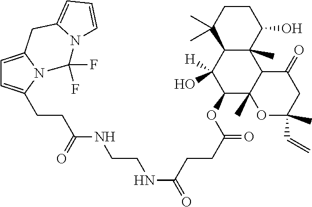 [SMILES: C=C[C@@]1(C)CC(=O)C2[C@@]3(C)[C@@H](O)CCC(C)(C)[C@@H]3[C@H](O)[C@H](OC(=O)CCC(=O)NCCNC(=O)CCc3ccc4n3C(F)(F)n3cccc3C4)[C@@]2(C)O1] | 1,9-dd-FS | 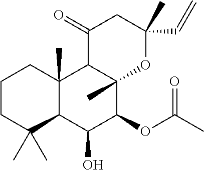 |
| 6A7DA-FS | 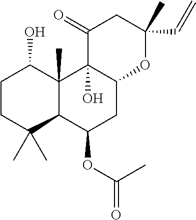 | calmidazolium | 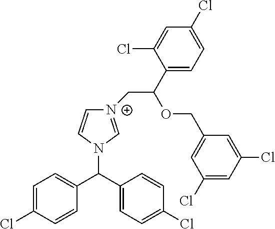 [SMILES: Clc1ccc(C(c2ccc(Cl)cc2)n2cc[n+](CC(OCc3cc(Cl)cc(Cl)c3)c3ccc(Cl)cc3Cl)c2)cc1] |
| Tyrphostin A25 | 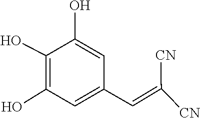 [SMILES: N#CC(C#N)=Cc1cc(O)c(O)c(O)c1] | 9- Cyclopentyl- adenine monomethane- sulfonate | 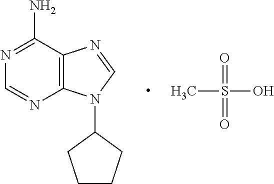 |
| (E)-2-(1H- Benzo[d]imida- zol-2-ylthio)-N′- (5-bromo-2- hydroxybenzyl- idene)propane- hydrazide | 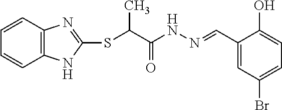 [SMILES: CC(Sc1nc2ccccc2[nH]1)C(=O)N/N=C/c1cc(Br)ccc1O] | SB-268262 | 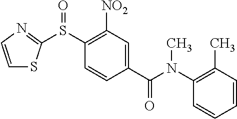 [SMILES: Cc1ccccc1N(C)C(=O)c1ccc(S(=O)c2nccs2)c([N+](=O)[O-])c1] |
| LRE1 | 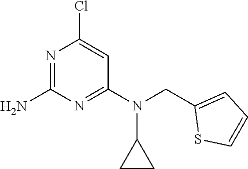 [SMILES: Nc1nc(Cl)cc(N(Cc2cccs2)C2CC2)n1] | 2′,5′- Dideoxy- adenosine | 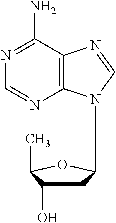 |
| 2′,5′- Dideoxy- adenosine 3′- triphosphate tetrasodium salt | 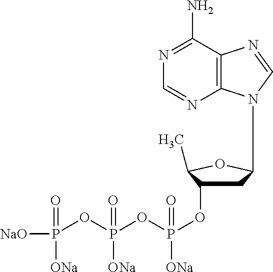 | | |

Additional ADCY inhibitors useful in the compositions and methods of the present invention are disclosed in Dessauer et al. Pharmacol Rev, (2017) 69 (2): 93-139, and have the structure:

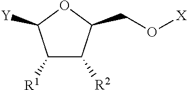

| Compound | R1 | R2 | X | Y |
|---|---|---|---|---|
| MANT- ATP | 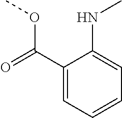 | OH | 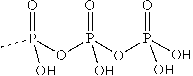 | 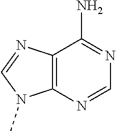 |
| MANT-ITP | 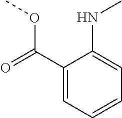 | OH | 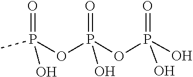 | 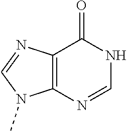 |
| MANT- GTP | 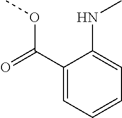 | OH | 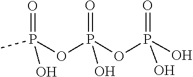 | 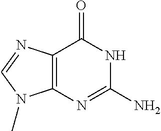 |
| MANT- XTP | 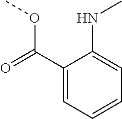 | OH | 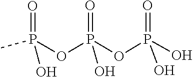 | 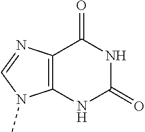 |
| MANT- CTP | 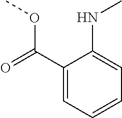 | OH | 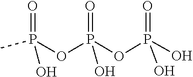 | 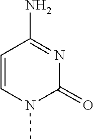 |
| MANT- UTP | 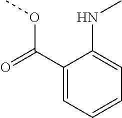 | OH | 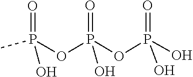 | 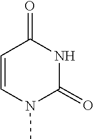 |
| 2′-MANT- 3′dATP | H | 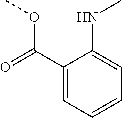 | 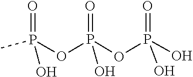 | 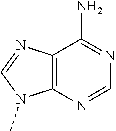 |
| 3′-MANT- 2′dATP | 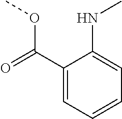 | H | 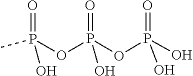 | 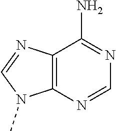 |
| MANT- ATPγS | 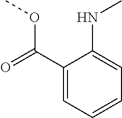 | OH | 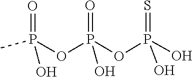 | 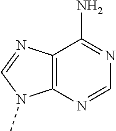 |
| MANT- ITPγS | 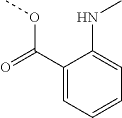 | OH | 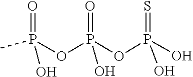 | 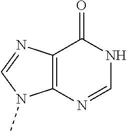 |
| MANT- GTPγS | 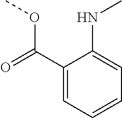 | OH | 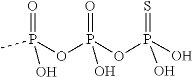 | 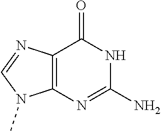 |
| MANT- UTPγS | 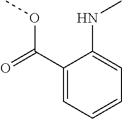 | OH | 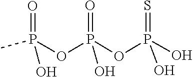 | 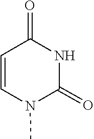 |
| ANT-ATP | 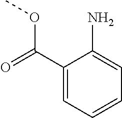 | OH | 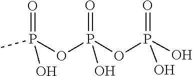 | 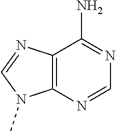 |
| Cl-ANT- ATP | 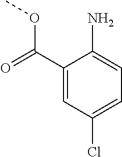 | OH | 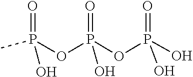 | 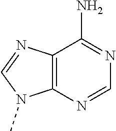 |
| Cl-ANT- ITP | 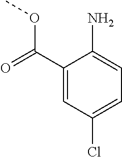 | OH | 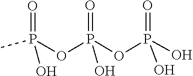 | 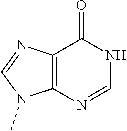 |
| Br-ANT- ITP | 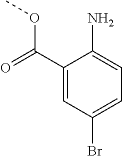 | OH | 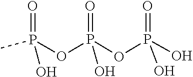 | 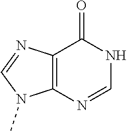 |
| Pr-ANT- ATP | 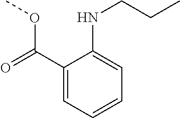 | OH | 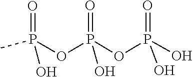 | 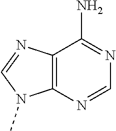 |
| Pr-ANT- ITP | 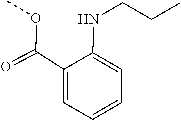 | OH | 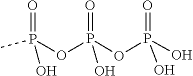 | 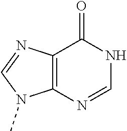 |
| AcNH- ANT-ATP | 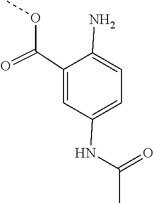 | OH | 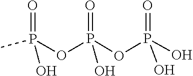 | 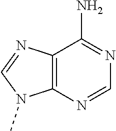 |
| AcNH- ANT-ITP | 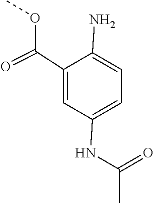 | OH | 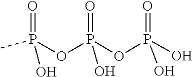 | 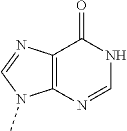 |
| MANT- AppNHp | 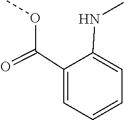 | OH | 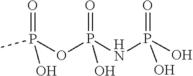 | 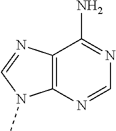 |
| MANT- GppNHp | 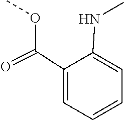 | OH | 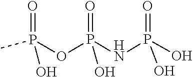 | 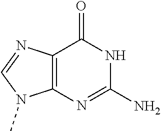 |
| TNP-ATP | 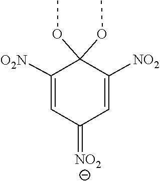 | | 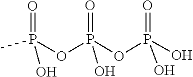 | 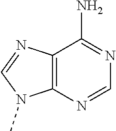 |
| TNP-GTP | 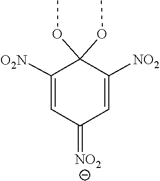 | | 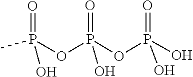 | 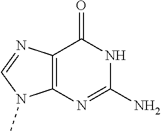 |
| TNP-CTP | 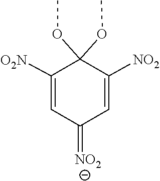 | | 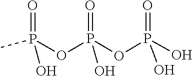 | 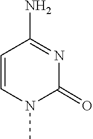 |
| TNP-UTP | 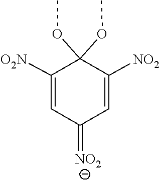 | | 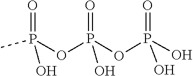 | 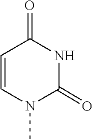 |
| Bis- MANT-ATP | 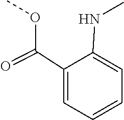 | 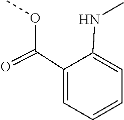 | 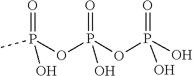 | 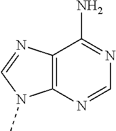 |
| Bis- MANT-ITP | 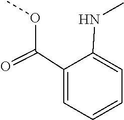 | 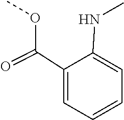 | 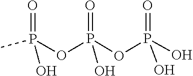 | 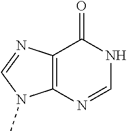 |
| Bis- MANT- CTP | 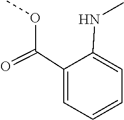 | 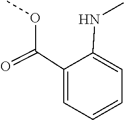 | 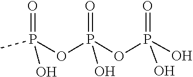 | 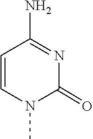 |
| Bis- MANT- IDP | 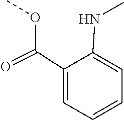 | 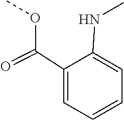 | 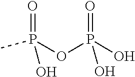 | 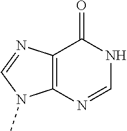 |
| Bis- MANT- IMP | 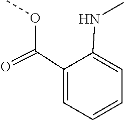 | 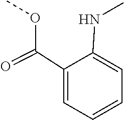 | 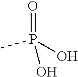 | 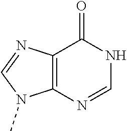 |
| Bis-Cl- ANT-ATP | 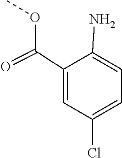 | 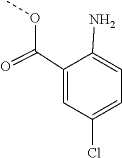 | 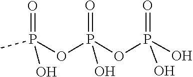 | 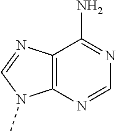 |
| Bis-Cl- ANT-ITP | 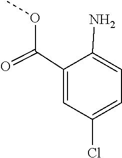 | 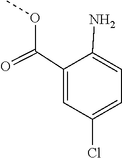 | 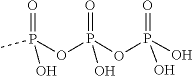 | 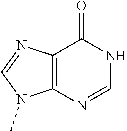 |
| Bis-Br- ANT-ATP | 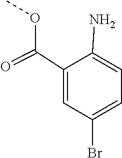 | 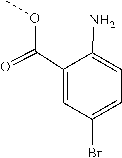 | 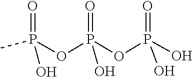 | 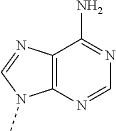 |
| Bis-Br- ANT-ITP | 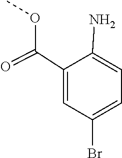 | 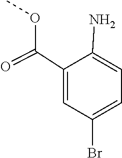 | 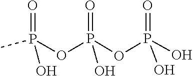 | 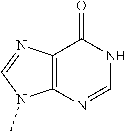 |
| Bis-Pr- ANT-ATP | 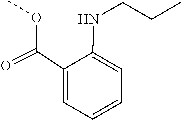 | 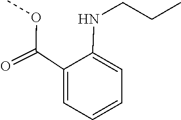 | 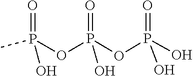 | 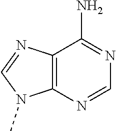 |
| Bis-Pr- ANT-ITP | 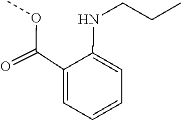 | 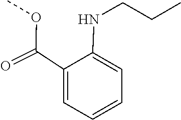 | 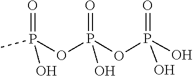 | 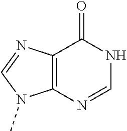 |
| Bis-AcNH- ANT-ATP | 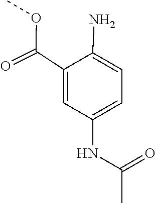 | 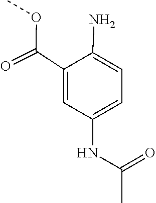 | 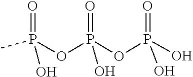 | 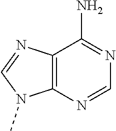 |
| Bis-AcNH- ANT-ITP | 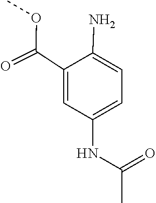 | 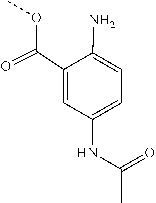 | 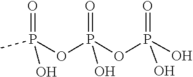 | 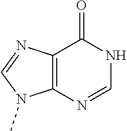 |

and pharmaceutically acceptable salts of the foregoing.

Additional examples of small molecule ADCY inhibitors include, but are not limited to: SQ22536 (9-(tetrahydro-2-furanyl)-adenine); 2′,5′-dideoxyadenosine, 9-cyclopentyladenine; 2′,5′-dideoxyadenosine 3′-diphosphate; 2′,5′-dideoxyadenosine 3′-monophosphate; MDL-12330A (cis-N-(2-phenylcyclopentyl)azacyclotridece-1-en-2-amine); 2-amino-7-(4-chlorophenyl)-7,8-dihydro-5 (6H)-quinazolinone; 2-amino-7-(4-methoxyphenyl)-7,8-dihydro-5(6H)-quinazolinone; 2-amino-7-phenyl-7,8-dihydro-5(6H)-quinazolinone; 4.2-amino-7-(2-furanyl)-7,8-dihydro-5(6H)-quinazolinone; 2-amino-7-(2-thienyl)-7,8-dihydro-5(6H)-quinazolinone); MANT-ATP; MANT-ITP; MANT-GTP; MANT-XTP; MANT-CTP; MANT-UTP; 2′-MANT-3′dATP; 3′-MANT-2′dATP; MANT-ATPTS; MANT-ITPTS; MANT-GTPTS; MANT-UTPTS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-IMP; Bis-Cl-ANT-ATP; Bis-Cl-ANT-ITP; Bis-Br-ANT-ATP; Bis-Br-ANT-ITP; Bis-Pr-ANT-ATP; Bis-Pr-ANT-ITP; Bis-AcNH-ANT-ATP; Bis-AcNH-ANT-ITP; NKY80; vidarabine; 2′,5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; and 2′,5′-Dideoxyadenosine 3′-triphosphate tetrasodium salt; and pharmaceutically acceptable salts of the foregoing.

Illustrative ADCY inhibitor peptides useful in the compositions and methods of the present invention include, but are not limited to: adrenocorticotropic hormone; brain natriuretic peptide (BNP); and pituitary adenylate cyclase-activating polypeptide.

### Pharmaceutically Acceptable Salts

Pharmaceutically acceptable salts include, for example, acid-addition salts and base-addition salts. The acid that forms an acid-addition salt can be an organic acid or an inorganic acid. A base that forms a base-addition salt can be an organic base or an inorganic base. In some embodiments, a pharmaceutically acceptable salt is a metal salt. In some embodiments, a pharmaceutically acceptable salt is an ammonium salt.

Acid-addition salts can arise from the addition of an acid to the free-base form of a compound useful in the compositions and methods of the invention. In some embodiments, the acid is organic. In some embodiments, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid-addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to a compound having a carboxyl group. The inorganic base can include a metal cation paired with a basic counterion, such as, for example, hydroxide, carbonate, bicarbonate, or phosphate. The metal can be an alkali metal, alkaline earth metal, transition metal, or main group metal. Non-limiting examples of suitable metals include lithium, sodium, potassium, cesium, cerium, magnesium, manganese, iron, calcium, strontium, cobalt, titanium, aluminum, copper, cadmium, and zinc.

Non-limiting examples of suitable metal salts include a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, and a zinc salt.

Ammonium salts can arise from the addition of ammonia or an organic amine to a compound having a carboxyl group. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include a triethylammonium salt, a diisopropylammonium salt, an ethanolammonium salt, a diethanolammonium salt, a triethanolammonium salt, a morpholinium salt, an N-methylmorpholinium salt, a piperidinium salt, an N-methylpiperidinium salt, an N-ethylpiperidinium salt, a dibenzylammonium salt, a piperazinium salt, a pyridinium salt, a pyrrazolium salt, an imidazolium salt, a pyrazinium salt, an ethylenediammonium salt, an N,N′-dibenzylethylenediammonium salt, a procaine salt, a chloroprocaine salt, a choline salt, a dicyclohexylammonium salt, and a N-methylglucamine salt.

### ADCY9 Gene Genotype

The present invention refers to the following nucleotide and amino acid sequences: The sequences provided herein are available in the NCBI database and can be retrieved from www.ncbi.nlm.nih.gov/sites/entrez?db+gene; Theses sequences also relate to annotated and modified sequences. The present invention also provides techniques and methods wherein homologous sequences, and variants of the concise sequences provided herein are used.

Preferably, such “variants” are genetic variants. ON NCBI database the Nucleotide sequence encoding Homo sapiens Adenylate Cyclase Type 9 (ACDY9) is available. Homo sapiens Adenylate Cyclase Type 9(ADCY9), RefSeqGeneon chromosome 16 NCBI Reference Sequence: NCBI accession number NG 011434.1 Homo sapiens chromosome 16 genomic contig, GRCh3 7.p10 Primary Assembly NCBI Reference Sequence: NCBI accession number NT_010393.16. The intronic sequences for Homo sapiens ACDY9 gene SNPs providing the “rs” designation, alleles and corresponding SEQ ID number designations is disclosed in Tables 1, 2 and 3. The polymorphisms are identified in bold and within bracket.

TABLE 1
ACDY9 SNPs and respective intronic sequence

| | SEQ. | Intronic | |
|---|---|---|---|
| | ID. | | |
| SNP rs ID | NO.: | sequence¹ | HGVS Names |
| rs11647778 | 21 | GGACCTGCCTGGTG | NC_0000 16.10:g.4001379C>G |
| | | CTTTCTCAGAG[C/G] | NG_011434.1:g.119807G>C |
| | | AGACTGAGGTTTGG | NM_001116.3:c.1884+5989G> |
| | | GGTTTGCGGAA | NT_110393.17:g.3991379C>G |
| rs1967309 | 20 | TTAACCTATTTATTT | NC_000016.9:g.4065583A>G |
| | | CTTTCAACCCT[C/T] | NG_011434.1:g.105604T>C |
| | | AGCCCAGATCCTAA | NM_001116.3:c.1694−8024T>C |
| | | CCTTCGGTAAG | NT_010393.16:g.4005583A>G |
| rs12595857 | 2 | CATTGATTTT AAAC | NC_000016.9:g.4062592G>A |
| | | CTCAACAACAGC | NG_011434.1:g.108595C>T |
| | | [A/G]ATGTCTTTTA | NM_001116.3:c.1694−5033C>T |
| | | TCAGCTTAATTTTAC | NT_010393.16:g.4002592G>A |

¹Source from NCBI Genome reference Build 37.3

TABLE 2
List of genetic variants in gene ADCY9 on chr16 which have provided
evidence of association (P < 0.05) with response to treatment with
dalcetrapib from the GW AS study with reference sequence from
the genotyping chip used for the experiment (Illumina OMNI2.5S):

| | Position | SNPrs | | | SEQ. |
|---|---|---|---|---|---|
| | (GRCh37/ | identifier | | | ID |
| Chr. | hg19) | (NCBI) | P value | Sequence^{1, 2} | NO. |
| 16 | 4,065,583 | Rs1967309 | 4.11E−08 | TTCATGCACCCA | 1 |
| | | | | GCAGACTAAATG | |
| | | | | TTTACTGAGTAC | |
| | | | | TTACCGAAGGTT | |
| | | | | AGGATCTGGGCT | |
| | | | | [A/G]AGGGTYGA | |
| | | | | AAGAAATAAATA | |
| | | | | GGTTAAAAAAGA | |
| | | | | AAAAAAGCCACC | |
| | | | | TAGGTGACTTTC | |
| | | | | ACTC¹ | |
| 16 | 4,062,592 | rs12595857 | 4.53E−07 | TTAATATGATTT | 2 |
| | | | | CTTATATTCTTTC | |
| | | | | CTGGTTATCCAT | |
| | | | | TGATTTTAAACC | |
| | | | | TCAACAACAGC | |
| | | | | [A/G]ATGTCTTTT | |
| | | | | ATCAGCTTAATT | |
| | | | | TTACAAAGGCTA | |
| | | | | CAGAGAGGGGT | |
| | | | | GGGCATTTCCTA | |
| | | | | ATGG² | |
| 16 | 4,060,661 | rs2239310 | 1.29E−06 | CCTGTGTGGAGC | 3 |
| | | | | CCATTACCTGAA | |
| | | | | GAGGGGCCAAG | |
| | | | | AGGACAAGCAG | |
| | | | | GTATGACTATGG | |
| | | | | TC[A/G]GGCGTG | |
| | | | | CCAAGTCCCAGG | |
| | | | | ACAAGGAAGGA | |
| | | | | CGGGTGCTCCAG | |
| | | | | GAAGCACAGGA | |
| | | | | GGGGGCAT² | |
| 16 | 4,051,513 | rs11647828 | 2.76E−06 | TACCGGATGGCA | 4 |
| | | | | GTGAGCAGGGA | |
| | | | | GGCTCACCTGGA | |
| | | | | TCATTTGGTGAA | |
| | | | | GGTGGCATCTGC | |
| | | | | C[T/C]GGTTTGTC | |
| | | | | CACTGTGAAGTT | |
| | | | | CCTATTCCTACC | |
| | | | | CCGCCCCCCACC | |
| | | | | TTTCTTTTTTGAG | |
| | | | | ATG² | |
| 16 | 4,076,094 | rs8049452 | 6.63E−06 | ACTTAACTATTT | 5 |
| | | | | GTTGGGTGAATA | |
| | | | | TAGAAATGAATG | |
| | | | | AATGAATGGATG | |
| | | | | GATGAGCAGATA | |
| | | | | [T/C]ATCAAGAA | |
| | | | | GTTAATTCACAA | |
| | | | | ATTAAAGCCCAT | |
| | | | | TATGAAACTAAA | |
| | | | | GTAGAGGCTGGG | |
| | | | | CGCG¹ | |
| 16 | 4,049,365 | rs12935810 | 2.98E−05 | ACCCGTGAACAA | 6 |
| | | | | GTCGGGCCCCCA | |
| | | | | TCCACGCAATAT | |
| | | | | CTGCAGTCTCGA | |
| | | | | CTGTATGATCTC | |
| | | | | [A/G]TCCTTTGCA | |
| | | | | GCCACACTGTGA | |
| | | | | GGCAGCAATGAT | |
| | | | | CATTCCGCAGAC | |
| | | | | GGCCACAGACTC | |
| | | | | CAG² | |
| 16 | 4,065,495 | rs74702385 | 8.87E−05 | GACGACACCCAG | 7 |
| | | | | CACACCCAGCAC | |
| | | | | ACCCAGCACACC | |
| | | | | AGCGAACAGCCC | |
| | | | | ACCAGGTGCTAT | |
| | | | | [T/C]GCTGTCATT | |
| | | | | CATTTGCTCATT | |
| | | | | CGCTCGTTCATG | |
| | | | | CACCCAGCAGAC | |
| | | | | TAAATGTTTACT | |
| | | | | GAG¹ | |
| 16 | 4,076,047 | Rs17136707 | 9.11E−05 | AAAACAGTGCTC | 8 |
| | | | | CAAAGGCAAAG | |
| | | | | AAATAGCAAAG | |
| | | | | ACAGAAGTAAG | |
| | | | | GCACTTAACTAT | |
| | | | | TTG[T/C]TGGGTG | |
| | | | | AATATAGAAATG | |
| | | | | AATGAATGAATG | |
| | | | | GATGGATGAGCA | |
| | | | | GATACATCAAGA | |
| | | | | AGTTAA¹ | |
| 16 | 4,070,333 | rs8061182 | 1.51E−04 | GGCAGCTATGTA | 9 |
| | | | | GGAAGCAGTGA | |
| | | | | AGATCCACATCC | |
| | | | | TTCCTTATTGGT | |
| | | | | GAAAGGAATGA | |
| | | | | AT[T/C]GGAAAC | |
| | | | | AGAAAGTTCTTT | |
| | | | | TTTACCTTTATTA | |
| | | | | AATAAACGTGAA | |
| | | | | GTCATAAGAACT | |
| | | | | ACTAA² | |
| 16 | 4,064,368 | rs111590482 | 1.64E−04 | AGACTTTGTCTC | 10 |
| | | | | AAAAAAGAAAA | |
| | | | | AAAAAAAAAAA | |
| | | | | GAAGTCCCAAAT | |
| | | | | AATAAAATATGA | |
| | | | | GA[T/C]GGATTT | |
| | | | | ATGGAAGAAAGT | |
| | | | | GAAAGAAACAA | |
| | | | | AGGGTAGGCACC | |
| | | | | TTGCCTGTTTAA | |
| | | | | TTTGATC¹ | |
| 16 | 4,076,136 | rs4786454 | 1.98E−04 | TGGATGGATGAG | 11 |
| | | | | CAGATACATCAA | |
| | | | | GAAGTTAATTCA | |
| | | | | CAAATTAAAGCC | |
| | | | | CATTATGAAACT | |
| | | | | [A/G]AAGTAGAG | |
| | | | | GCTGGGCGCGGT | |
| | | | | GGATCACGCCTA | |
| | | | | TAATCCCAGCAC | |
| | | | | TTTGGGAGGTCA | |
| | | | | AGGC² | |
| 16 | 4,066,061 | rs2283497 | 8.87E−04 | TGTGATATGATG | 12 |
| | | | | GTCATATCATAG | |
| | | | | CACAGGGCTGTT | |
| | | | | GTGAGGATTAAA | |
| | | | | TGAGTTGATTCA | |
| | | | | [T/G]GTAAACAGG | |
| | | | | GACATCCGAAAA | |
| | | | | AGGGAAAGACG | |
| | | | | GTGCTTGTCCTG | |
| | | | | AGAACAGCTGTG | |
| | | | | AATG¹ | |
| 16 | 4,052,486 | rs2531967 | 1.11E−03 | AGGTGAGTGGCC | 13 |
| | | | | TTAAAGGGGAAG | |
| | | | | GAGAAACCTTTT | |
| | | | | GAAAGCAGGAC | |
| | | | | AGGTCCTCTCTG | |
| | | | | A[A/G]TCATCCCC | |
| | | | | GTATGGGTAAAT | |
| | | | | CTACATCACTAG | |
| | | | | CTTCATTACTGA | |
| | | | | CTGGTCCATGTA | |
| | | | | GAAA¹ | |
| 16 | 4,057,603 | rs3730119 | 0.0108 | CAGGTATGTCTT | 14 |
| | | | | CAAACCTATGAT | |
| | | | | GGATAAAAGTTA | |
| | | | | CAGTCAGCACAG | |
| | | | | ATTGAAAGCACC | |
| | | | | [A/G]TCTGTTGAA | |
| | | | | ACGCAGCTCCGT | |
| | | | | CTTGCTCTCTGG | |
| | | | | AGAGGACTCACT | |
| | | | | CCTGGAAAGTTG | |
| | | | | AGA² | |
| 16 | 4,077,178 | rs13337675 | 0.0377 | TGTAACCAAGTA | 15 |
| | | | | ACCAATGGTAAA | |
| | | | | CCTCTACAGGGT | |
| | | | | ATTAAGGCTCCA | |
| | | | | GAAAATTCTCTA | |
| | | | | [A/G]TCAGCCACT | |
| | | | | TGCTCCTGCTCG | |
| | | | | AGCCTGCTCCCA | |
| | | | | CTCCGTGGAGTG | |
| | | | | TACTTTCATTTCA | |
| | | | | GT¹ | |

Chr: chromosome number;
P value: for association with cardiovascular events (primary CETP composite event or unanticipated coronary revascularization) in patients treated with the inhibitor dalcetrapib;
¹Reference sequence from the 1000 Genomes public database, as presented in the ILLUMINA annotation file for the OMNI 2.5S Chip Human0mni25Exome−8v1 A.csv;
²Reference sequence from the dbSNP public database version 131 from NCBI, as presented in the ILLUMINA annotation file for the OMNI 2.SS Chip Human0mni25Exome−8v1 A.csv.

TABLE 3
List of additional genetic variants in gene ADCY9 on chr16:

| | | Distance | | | | | SEQ. |
|---|---|---|---|---|---|---|---|
| | | (bp) | | | | | ID |
| Variation | Location¹ | from a | r2' ¹ | D' ¹ | Column² | HGV Name s² | NO. |
| rs12920508 | 16:4066891 | 1308 | 0.952954 | 1 | TTTGGGGTGACG | NC_000016.9: | 16 |
| | | | | | AAAATGTAAAAT | g.4066891G>C | |
| | | | | | TA[C/G/T]GTTGT | NC_000016.9: | |
| | | | | | GGTGATGGTTGC | g.4066891G>T | |
| | | | | | ACAACACC | NG 011434.1: | |
| | | | | | | g.104296C>A | |
| | | | | | | NG_011434.1: | |
| | | | | | | g.104296C>G | |
| | | | | | | NM_001116.3: | |
| | | | | | | c.1694−9332C>A | |
| | | | | | | NM_001116.3: | |
| | | | | | | c.1694−9332C>G | |
| | | | | | | NT_010393.16: | |
| | | | | | | g.4006891G>C | |
| | | | | | | NT_010393.16: | |
| | | | | | | g.4006891G>T | |
| rs12599911 | 16:4062436 | 3147 | 0.908417 | 1 | GAATAACCACAC | NC_000016.9: | 17 |
| | | | | | ACATGGACCCTG | g.4062436G>T | |
| | | | | | GG[G/T]TCCAAG | NG_111434.1: | |
| | | | | | TTCATTAGAATG | g.108751C>A | |
| | | | | | GCTCTTT | NM_001116.3: | |
| | | | | | | c.1694−4877C>A | |
| | | | | | | NT_010393.16: | |
| | | | | | | g.4002436G>T | |
| rs2531971 | 16:4051261 | 14322 | 0.840627 | 0.973493 | AAGACAGAGGA | NC_000016.9: | 18 |
| | | | | | ACCCCCATAGGC | g.4051261C>A | |
| | | | | | TGG(G/T)GGTGA | NG_011434.1: | |
| | | | | | GCAGGGGGCATG | g.119926G>T | |
| | | | | | AGGGCTAA | NM_001116.3: | |
| | | | | | | c.1884+6108G>T | |
| | | | | | | NT_010393.16: | |
| | | | | | | g.3991261C>A | |
| rs2238448 | 16:4059439 | 6144 | 0.840582 | 0.973467 | TGTCCAACTATT | NC_000016.9: | 19 |
| | | | | | TCTTTCTTTCTTT | g.4059439T>C | |
| | | | | | T[C/T)TGAG | NG_011434.1: | |
| | | | | | ATGGGGGTCTCAC | g.111748A>G | |
| | | | | | TGTGTTGG | NM_001116.3: | |
| | | | | | | c.1694−1880A>G | |
| | | | | | | NT_010393.16: | |
| | | | | | | g.3999439T>C | |

References:
a. rs1967309
¹ Location r2 and D' values from the 1000 Genomes public database
²Reference sequence &HGV Names from the dbSNP public database version 137 from NCBI

### Methods for Delaying Occurrence of New-Onset Type 2 Diabetes

The present invention provides methods for delaying occurrence of new-onset type 2 diabetes, comprising administering an effective amount of a CETP inhibitor to a subject in need thereof and known to have in the subject's ADCY9 gene genotype rs1967309/AA, rs1967309/AG, rs12595857/GG, rs12595857/AG, rs111590482/AG, rs111590482/GG, rs11647828/GG, rs12935810/GG, rs11647828/AG, rs17136707/GG, rs17136707/AG, rs2239310/GG, rs2239310/AG, rs2283497/AA, rs2283497/CA, rs2531967/AA, rs2531967/GA, rs3730119/AA, rs3730119/GA, rs12920508/CG, rs12920508/GG, rs2531971/AC, rs2531971/AA, rs12599911/GT, rs12599911/GG, rs2238448/TC, rs2238448/TT, rs4786454/AA, rs4786454/GA, rs74702385/GA, rs74702385/AA, rs8049452/GG, rs8049452/GA, rs8061182/AG, rs8061182/AA, rs13337675/AG, rs13337675/GG, rs11647778/CG, or rs11647778/CC.

In some embodiments, the subject is known to have in the subject's ADCY9 gene genotype rs1967309/AA or rs1967309/AG.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the subject has an HbA1c level that is less than 6.5% of whole blood. In some embodiments, the subject has an HbA1c level ranging from 5.7% to 6.4% of whole blood. In some embodiments, the subject has a fasting plasma glucose level that is less than 126 mg/dL. In some embodiments, the subject has a fasting plasma glucose level ranging from 100 mg/dL to 125 mg/dL.

In some embodiments, the subject is a human. In some embodiments, the subject is an adult human. In some embodiments, the subject is a pediatric human.

The present invention also provides methods for delaying occurrence of new-onset type 2 diabetes, comprising administering to a subject in need thereof an effective amount of: (a) a CETP inhibitor; and (b) an ADCY inhibitor. In some embodiments, administering the CETP inhibitor occurs before, concurrently with, or after administering the ADCY inhibitor.

In some embodiments, the subject is known to have in the subject's ADCY9 gene genotype rs11647778/CC, rs12920508/GG, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs2531971/AA, rs8049452/GG, rs12599911/GG, rs8061182/AA or rs2238448/TT. In some embodiments, the subject is known to have in the subject's ADCY9 gene genotype rs1967309/AA.

In some embodiments, the subject is known to have in the subject's ADCY9 gene genotype 11647778/CG, rs12920508/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs2531971/AC, rs8049452/GA, rs12599911/GT, rs8061182/AG or rs2238448/TC. In some embodiments, the subject is known to have in the subject's ADCY9 gene genotype rs1967309/AG.

In some embodiments, the subject is known to have in the subject's ADCY9 gene genotype rs11647778/GG, rs12920508/CC, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs2531971/CC, rs8049452/AA, rs8061182/GG or rs2238448/CC. In some embodiments, the subject is known to have in the subject's ADCY9 gene genotype rs1967309/GG.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the subject has an HbA1c level that is less than 6.5% of whole blood. In some embodiments, the subject has an HbA1c level ranging from 5.7% to 6.4% of whole blood. In some embodiments, the subject has a fasting plasma glucose level that is less than 126 mg/dL. In some embodiments, the subject has a fasting plasma glucose level ranging from 100 mg/dL to 125 mg/dL.

In some embodiments, the subject is a human. In some embodiments, the subject is an adult human. In some embodiments, the subject is a pediatric human.

### Methods for Slowing Progression of Type 2 Diabetes

The present invention also provides methods for slowing progression of type 2 diabetes, comprising administering an effective amount of a CETP inhibitor to a subject in need thereof and known to have in the subject's ADCY9 gene genotype rs1967309/AA, rs1967309/AG, rs12595857/GG, rs12595857/AG, rs111590482/AG, rs111590482/GG, rs11647828/GG, rs12935810/GG, rs11647828/AG, rs17136707/GG, rs17136707/AG, rs2239310/GG, rs2239310/AG, rs2283497/AA, rs2283497/CA, rs2531967/AA, rs2531967/GA, rs3730119/AA, rs3730119/GA, rs12920508/CG, rs12920508/GG, rs2531971/AC, rs2531971/AA, rs12599911/GT, rs12599911/GG, rs2238448/TC, rs2238448/TT, rs4786454/AA, rs4786454/GA, rs74702385/GA, rs74702385/AA, rs8049452/GG, rs8049452/GA, rs8061182/AG, rs8061182/AA, rs13337675/AG, rs13337675/GG, rs11647778/CG, or rs11647778/CC.

In some embodiments, the subject is known to have in the subject's ADCY9 gene genotype rs1967309/AA or rs1967309/AG.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the methods further comprise administering to the subject an antidiabetic agent. In some embodiments, the subject undergoes treatment with an antidiabetic agent. In some embodiments, the amount of antidiabetic agent administered is an effective amount. In some embodiments, the total amount of CETP inhibitor and antidiabetic agent administered is an effective amount.

In some embodiments, the antidiabetic agent is metformin or a pharmaceutically acceptable salt thereof.

In some embodiments, the antidiabetic agent is a sulfonylurea. In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analogue is exenatide, liraglutide, lixisenatide, albiglutide, dulaglutide or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin or dutogliptin, or a pharmaceutically acceptable salt of any of the foregoing.

In some embodiments, the subject has an HbA1c level that is equal to or greater than 6.5% of whole blood. In some embodiments, the subject has an HbA1c level ranging from 6.5% to 20% of whole blood. In some embodiments, the subject has an HbA1c level that is equal to or greater than 7.0% of whole blood. In some embodiments, the subject has an HbA1c level ranging from 7.0% to 20% of whole blood. In some embodiments, the subject has an HbA1c level that is equal to or greater than 7.5% of whole blood. In some embodiments, the subject has an HbA1c level ranging from 7.5% to 20% of whole blood.

In some embodiments, the subject has a fasting plasma glucose level that is equal to or greater than 126 mg/dL. In some embodiments, the subject has a fasting plasma glucose level ranging from 126 mg/dL to 600 mg/dL.

In some embodiments, the subject is a human. In some embodiments, the subject is an adult human. In some embodiments, the subject is a pediatric human.

The present invention also provides methods for slowing progression of type 2 diabetes, comprising administering to a subject in need thereof an effective amount of: (a) a CETP inhibitor; and (b) an ADCY inhibitor. In some embodiments, administering the CETP inhibitor occurs before, concurrently with, or after administering the ADCY inhibitor.

In some embodiments, the subject is known to have in the subject's ADCY9 gene genotype rs11647778/CC, rs12920508/GG, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs2531971/AA, rs8049452/GG, rs12599911/GG, rs8061182/AA or rs2238448/TT. In some embodiments, the subject is known to have in the subject's ADCY9 gene genotype rs1967309/AA.

In some embodiments, the subject is known to have in the subject's ADCY9 gene genotype 11647778/CG, rs12920508/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs2531971/AC, rs8049452/GA, rs12599911/GT, rs8061182/AG or rs2238448/TC. In some embodiments, the subject is known to have in the subject's ADCY9 gene genotype rs1967309/AG.

In some embodiments, the subject is known to have in the subject's ADCY9 gene genotype rs11647778/GG, rs12920508/CC, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs2531971/CC, rs8049452/AA, rs8061182/GG or rs2238448/CC. In some embodiments, the subject is known to have in the subject's ADCY9 gene genotype rs1967309/GG.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the methods further comprise administering to the subject an antidiabetic agent. In some embodiments, the subject undergoes treatment with an antidiabetic agent. In some embodiments, the amount of antidiabetic agent administered is an effective amount. In some embodiments, the total amount of CETP inhibitor, ADCY inhibitor and antidiabetic agent administered is an effective amount.

In some embodiments, the antidiabetic agent is metformin or a pharmaceutically acceptable salt thereof.

In some embodiments, the antidiabetic agent is a sulfonylurea. In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analogue is exenatide, liraglutide, lixisenatide, albiglutide, dulaglutide or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin or dutogliptin, or a pharmaceutically acceptable salt of any of the foregoing.

In some embodiments, the subject has an HbA1c level that is equal to or greater than 6.5% of whole blood. In some embodiments, the subject has an HbA1c level ranging from 6.5% to 20% of whole blood. In some embodiments, the subject has an HbA1c level that is equal to or greater than 7.0% of whole blood. In some embodiments, the subject has an HbA1c level ranging from 7.0% to 20% of whole blood. In some embodiments, the subject has an HbA1c level that is equal to or greater than 7.5% of whole blood. In some embodiments, the subject has an HbA1c level ranging from 7.5% to 20% of whole blood.

In some embodiments, the subject has a fasting plasma glucose level that is equal to or greater than 126 mg/dL. In some embodiments, the subject has a fasting plasma glucose level ranging from 126 mg/dL to 600 mg/dL.

In some embodiments, the subject is a human. In some embodiments, the subject is an adult human. In some embodiments, the subject is a pediatric human.

### Methods for Treating Type 2 Diabetes

The present invention further provides methods for treating type 2 diabetes, comprising administering an effective amount of a CETP inhibitor to a subject in need thereof and known to have in the subject's ADCY9 gene genotype rs1967309/AA, rs1967309/AG, rs12595857/GG, rs12595857/AG, rs111590482/AG, rs111590482/GG, rs11647828/GG, rs12935810/GG, rs11647828/AG, rs17136707/GG, rs17136707/AG, rs2239310/GG, rs2239310/AG, rs2283497/AA, rs2283497/CA, rs2531967/AA, rs2531967/GA, rs3730119/AA, rs3730119/GA, rs12920508/CG, rs12920508/GG, rs2531971/AC, rs2531971/AA, rs12599911/GT, rs12599911/GG, rs2238448/TC, rs2238448/TT, rs4786454/AA, rs4786454/GA, rs74702385/GA, rs74702385/AA, rs8049452/GG, rs8049452/GA, rs8061182/AG, rs8061182/AA, rs13337675/AG, rs13337675/GG, rs11647778/CG, or rs11647778/CC.

In some embodiments, the subject is known to have in the subject's ADCY9 gene genotype rs1967309/AA or rs1967309/AG.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the methods further comprise administering to the subject an antidiabetic agent. In some embodiments, the subject undergoes treatment with an antidiabetic agent. In some embodiments, the amount of antidiabetic agent administered is an effective amount. In some embodiments, the total amount of CETP inhibitor and antidiabetic agent administered is an effective amount.

In some embodiments, the antidiabetic agent is metformin or a pharmaceutically acceptable salt thereof.

In some embodiments, the antidiabetic agent is a sulfonylurea. In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analogue is exenatide, liraglutide, lixisenatide, albiglutide, dulaglutide or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin or dutogliptin, or a pharmaceutically acceptable salt of any of the foregoing.

In some embodiments, the subject has an HbA1c level that is equal to or greater than 6.5% of whole blood. In some embodiments, the subject has an HbA1c level ranging from 6.5% to 20% of whole blood. In some embodiments, the subject has an HbA1c level that is equal to or greater than 7.0% of whole blood. In some embodiments, the subject has an HbA1c level ranging from 7.0% to 20% of whole blood. In some embodiments, the subject has an HbA1c level that is equal to or greater than 7.5% of whole blood. In some embodiments, the subject has an HbA1c level ranging from 7.5% to 20% of whole blood.

In some embodiments, the subject has a fasting plasma glucose level that is equal to or greater than 126 mg/dL. In some embodiments, the subject has a fasting plasma glucose level ranging from 126 mg/dL to 600 mg/dL.

In some embodiments, the subject is a human. In some embodiments, the subject is an adult human. In some embodiments, the subject is a pediatric human.

The present invention further provides methods for treating type 2 diabetes, comprising administering to a subject in need thereof an effective amount of: (a) a CETP inhibitor; and (b) an ADCY inhibitor. In some embodiments, administering the CETP inhibitor occurs before, concurrently with, or after administering the ADCY inhibitor.

In some embodiments, the subject is known to have in the subject's ADCY9 gene genotype rs11647778/CC, rs12920508/GG, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs2531971/AA, rs8049452/GG, rs12599911/GG, rs8061182/AA or rs2238448/TT. In some embodiments, the subject is known to have in the subject's ADCY9 gene genotype rs1967309/AA.

In some embodiments, the subject is known to have in the subject's ADCY9 gene genotype 11647778/CG, rs12920508/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs2531971/AC, rs8049452/GA, rs12599911/GT, rs8061182/AG or rs2238448/TC. In some embodiments, the subject is known to have in the subject's ADCY9 gene genotype rs1967309/AG.

In some embodiments, the subject is known to have in the subject's ADCY9 gene genotype rs11647778/GG, rs12920508/CC, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs2531971/CC, rs8049452/AA, rs8061182/GG or rs2238448/CC. In some embodiments, the subject is known to have in the subject's ADCY9 gene genotype rs1967309/GG.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the methods further comprise administering to the subject an antidiabetic agent. In some embodiments, the subject undergoes treatment with an antidiabetic agent. In some embodiments, the amount of antidiabetic agent administered is an effective amount. In some embodiments, the total amount of CETP inhibitor, ADCY inhibitor and antidiabetic agent administered is an effective amount.

In some embodiments, the antidiabetic agent is metformin or a pharmaceutically acceptable salt thereof.

In some embodiments, the antidiabetic agent is a sulfonylurea. In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analogue is exenatide, liraglutide, lixisenatide, albiglutide, dulaglutide or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin or dutogliptin, or a pharmaceutically acceptable salt of any of the foregoing.

In some embodiments, the subject has an HbA1c level that is equal to or greater than 6.5% of whole blood. In some embodiments, the subject has an HbA1c level ranging from 6.5% to 20% of whole blood. In some embodiments, the subject has an HbA1c level that is equal to or greater than 7.0% of whole blood. In some embodiments, the subject has an HbA1c level ranging from 7.0% to 20% of whole blood. In some embodiments, the subject has an HbA1c level that is equal to or greater than 7.5% of whole blood. In some embodiments, the subject has an HbA1c level ranging from 7.5% to 20% of whole blood.

In some embodiments, the subject has a fasting plasma glucose level that is equal to or greater than 126 mg/dL. In some embodiments, the subject has a fasting plasma glucose level ranging from 126 mg/dL to 600 mg/dL.

In some embodiments, the subject is a human. In some embodiments, the subject is an adult human. In some embodiments, the subject is a pediatric human.

### Methods for Slowing Progression of a Complication of Type 2 Diabetes

The present invention still further provides methods for slowing progression of a complication of type 2 diabetes, comprising administering an effective amount of a CETP inhibitor to a subject in need thereof and known to have in the subject's ADCY9 gene genotype rs1967309/AA, rs1967309/AG, rs12595857/GG, rs12595857/AG, rs111590482/AG, rs111590482/GG, rs11647828/GG, rs12935810/GG, rs11647828/AG, rs17136707/GG, rs17136707/AG, rs2239310/GG, rs2239310/AG, rs2283497/AA, rs2283497/CA, rs2531967/AA, rs2531967/GA, rs3730119/AA, rs3730119/GA, rs12920508/CG, rs12920508/GG, rs2531971/AC, rs2531971/AA, rs12599911/GT, rs12599911/GG, rs2238448/TC, rs2238448/TT, rs4786454/AA, rs4786454/GA, rs74702385/GA, rs74702385/AA, rs8049452/GG, rs8049452/GA, rs8061182/AG, rs8061182/AA, rs13337675/AG, rs13337675/GG, rs11647778/CG, or rs11647778/CC.

In some embodiments, the subject is known to have in the subject's ADCY9 gene genotype rs1967309/AA or rs1967309/AG.

In some embodiments, the complication of type 2 diabetes is a cardiovascular complication. In some embodiments, the cardiovascular complication is heart disease, hypertension, or stroke. In some embodiments, the heart disease is myocardial infarction or heart failure.

In some embodiments, the complication of type 2 diabetes is a renal complication. In some embodiments, the renal complication is nephropathy or kidney failure.

In some embodiments, the complication of type 2 diabetes is a neurological complication. In some embodiments, the neurological complication is neuropathy. In some embodiments, the neuropathy is peripheral neuropathy, autonomic neuropathy, neuropathic arthropathy, cranial neuropathy, compression mononeuropathy, femoral neuropathy, focal neuropathy, thoracic radiculopathy or unilateral foot drop.

In some embodiments, the complication of type 2 diabetes is an ophthalmological complication. In some embodiments, the ophthalmological complication is glaucoma, a cataract, nonproliferative retinopathy, proliferative retinopathy or macular edema.

In some embodiments, the complication of type 2 diabetes is a foot-related complication. In some embodiments, the foot-related complication is peripheral neuropathy, foot skin dryness, a callus, a foot ulcer, poor circulation or amputation.

In some embodiments, the complication of type 2 diabetes is a mental health-related complication. In some embodiments, the mental health-related complication is anger, denial, depression, stress or diabetes distress.

In some embodiments, the complication of type 2 diabetes is a pregnancy-related complication. In some embodiments, the pregnancy-related complication is a birth defect, premature delivery, miscarriage, macrosomia, hypoglycemia, infection, preeclampsia, jaundice or respiratory distress syndrome.

In some embodiments, the complication of type 2 diabetes is a dermatological complication. In some embodiments, the dermatological complication is a bacterial infection, a fungal infection, itching, acanthosis nigricans, diabetic dermopathy, necrobiosis lipoidica diabeticorum, an allergic skin reaction, bullosis diabeticorum, eruptive xanthomatosis, digital sclerosis or disseminated granuloma annulare.

In some embodiments, the complication of type 2 diabetes is diabetic ketoacidosis (DKA), hyperosmolar hyperglycemic nonketotic syndrome (HNS), hepatitis B infection, human immunodeficiency virus infection, adhesive capsulitis, hemochromatosis, sleep apnea, or gastroparesis.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the method further comprises administering to the subject an antidiabetic agent. In some embodiments, the subject undergoes treatment with an antidiabetic agent. In some embodiments, the amount of antidiabetic agent administered is an effective amount. In some embodiments, the total amount of CETP inhibitor and antidiabetic agent administered is an effective amount.

In some embodiments, the antidiabetic agent is metformin or a pharmaceutically acceptable salt thereof.

In some embodiments, the antidiabetic agent is a sulfonylurea. In some embodiments, the sulfonylureasulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analogue is exenatide, liraglutide, lixisenatide, albiglutide, dulaglutide, or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir, or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin, or dutogliptin, or a pharmaceutically acceptable salt of any of the foregoing.

In some embodiments, the subject has an HbA1c level that is equal to or greater than 6.5% of whole blood. In some embodiments, the subject has an HbA1c level ranging from 6.5% to 20% of whole blood. In some embodiments, the subject has an HbA1c level that is equal to or greater than 7.0% of whole blood. In some embodiments, the subject has an HbA1c level ranging from 7.0% to 20% of whole blood. In some embodiments, the subject has an HbA1c level that is equal to or greater than 7.5% of whole blood. In some embodiments, the subject has an HbA1c level ranging from 7.5% to 20% of whole blood.

In some embodiments, the subject has a fasting plasma glucose level that is equal to or greater than 126 mg/dL. In some embodiments, the subject has a fasting plasma glucose level ranging from 126 mg/dL to 600 mg/dL.

In some embodiments, the subject is an adult human. In some embodiments, the subject is a pediatric human.

In some embodiments, the CETP inhibitor of the methods of the invention is dalcetrapib or a pharmaceutically acceptable salt thereof.

The present invention also provides methods for slowing progression of type 2 diabetes, comprising administering to a subject in need thereof an effective amount of: (a) a CETP inhibitor; and (b) an ADCY inhibitor. In some embodiments, administering the CETP inhibitor occurs before, concurrently with, or after administering the ADCY inhibitor.

In some embodiments, the subject is known to have in the subject's ADCY9 gene genotype rs11647778/CC, rs12920508/GG, rs12595857/GG, rs1967309/AA, rs111590482/AG, rs111590482/GG, rs11647828/GG, rs12935810/GG, rs17136707/GG, rs2239310/GG, rs2283497/AA, rs2531967/AA, rs3730119/AA, rs4786454/AA, rs74702385/GA, rs74702385/AA, rs2531971/AA, rs8049452/GG, rs12599911/GG, rs8061182/AA or rs2238448/TT. In some embodiments, the subject is known to have in the subject's ADCY9 gene genotype rs1967309/AA.

In some embodiments, the subject is known to have in the subject's ADCY9 gene genotype 11647778/CG, rs12920508/CG, rs12595857/AG, rs13337675/AG, rs13337675/GG, rs1967309/AG, rs11647828/AG, rs17136707/AG, rs2239310/AG, rs2283497/CA, rs2531967/GA, rs3730119/GA, rs4786454/GA, rs2531971/AC, rs8049452/GA, rs12599911/GT, rs8061182/AG or rs2238448/TC. In some embodiments, the subject is known to have in the subject's ADCY9 gene genotype rs1967309/AG.

In some embodiments, the subject is known to have in the subject's ADCY9 gene genotype rs11647778/GG, rs12920508/CC, rs12595857/AA, rs13337675/AA, rs1967309/GG, rs111590482/AA, rs11647828/AA, rs12935810/GA, rs12935810/AA, rs17136707/AA, rs2239310/AA, rs2283497/CC, rs2531967/GG, rs3730119/GG, rs4786454/GG, rs74702385/GG, rs2531971/CC, rs8049452/AA, rs8061182/GG or rs2238448/CC. In some embodiments, the subject is known to have in the subject's ADCY9 gene genotype rs1967309/GG.

In some embodiments, administering the CETP inhibitor does not increase the subject's risk of a cardiovascular event. In some embodiments, administering the CETP inhibitor lowers the subject's risk of a cardiovascular event. In some embodiments, the cardiovascular event is coronary heart disease, cardiac arrest, myocardial infarction, ischemic stroke, congestive heart failure, sudden cardiac death, cerebral infarction, syncope, transient ischemic attack, angina or coronary revascularization. In some embodiments, the cardiac arrest is resuscitated cardiac arrest. In some embodiments, the myocardial infarction is non-fatal myocardial infarction. In some embodiments, the ischemic stroke is non-fatal ischemic stroke. In some embodiments, the angina is unstable angina. In some embodiments, the coronary revascularization is unanticipated coronary revascularization.

In some embodiments, the CETP inhibitor is administered to the subject in an amount ranging from 5 mg to 2400 mg per day. In some embodiments, the CETP inhibitor is administered to the subject in an amount ranging from 100 mg to 2400 mg per day. In some embodiments, the CETP inhibitor is administered to the subject in an amount of about 5 mg, 10 mg, 20 mg, 40 mg, 60 mg, 80 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily. In some embodiments, the CETP inhibitor is administered to the subject in an amount ranging from 100 mg to 1800 mg per day. In some embodiments, the CETP inhibitor is administered to the subject in an amount ranging from 300 mg to 900 mg per day. In some embodiments, the CETP inhibitor is administered to the subject in an amount of 600 mg per day.

In some embodiments, the methods further comprise administering to the subject an antidiabetic agent. In some embodiments, the subject undergoes treatment with an antidiabetic agent. In some embodiments, the amount of antidiabetic agent administered is an effective amount. In some embodiments, the total amount of CETP inhibitor, ADCY inhibitor and antidiabetic agent administered is an effective amount.

In some embodiments, the antidiabetic agent is metformin or a pharmaceutically acceptable salt thereof.

In some embodiments, the antidiabetic agent is a sulfonylurea. In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analogue is exenatide, liraglutide, lixisenatide, albiglutide, dulaglutide or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin or dutogliptin, or a pharmaceutically acceptable salt of any of the foregoing.

In some embodiments, the subject has an HbA1c level that is equal to or greater than 6.5% of whole blood. In some embodiments, the subject has an HbA1c level ranging from 6.5% to 20% of whole blood. In some embodiments, the subject has an HbA1c level that is equal to or greater than 7.0% of whole blood. In some embodiments, the subject has an HbA1c level ranging from 7.0% to 20% of whole blood. In some embodiments, the subject has an HbA1c level that is equal to or greater than 7.5% of whole blood. In some embodiments, the subject has an HbA1c level ranging from 7.5% to 20% of whole blood.

In some embodiments, the subject has a fasting plasma glucose level that is equal to or greater than 126 mg/dL. In some embodiments, the subject has a fasting plasma glucose level ranging from 126 mg/dL to 600 mg/dL.

In some embodiments, the subject is a human. In some embodiments, the subject is an adult human. In some embodiments, the subject is a pediatric human.

In some embodiments, CETP inhibitor of the methods of the invention is dalcetrapib or a pharmaceutically acceptable salt thereof.

### Dosages

The dosage of the CETP inhibitors, ADCY inhibitors and antidiabetic agents useful in the methods and compositions of the invention can be selected in accordance with a variety of factors including type, species, age, weight, sex and medical condition of the subject; the severity of the subject's disorder; the route of administration; the renal or hepatic function of the subject; or the CETP inhibitor, ADCY inhibitor or antidiabetic agent to be administered.

In some embodiments, the daily dosage amount of CETP inhibitor, ADCY inhibitor or antidiabetic agent useful in the methods and compositions of the invention ranges from about 1 mg to about 2400 mg.

In certain embodiments, the CETP inhibitor is dalcetrapib or a pharmaceutically acceptable salt thereof, and the dalcetrapib or pharmaceutically acceptable salt thereof is administered orally at an amount of about 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, or 2400 mg daily.

In certain embodiments, the CETP inhibitor is torcetrapib or a pharmaceutically acceptable salt thereof, and the torcetrapib or pharmaceutically acceptable salt thereof is administered orally at a dose of about 20 mg, 30 mg, 40 mg, 50 mg, 60 mg, 70 mg, 80 mg, 90 mg, or 100 mg daily.

In certain embodiments, the CETP inhibitor is anacetrapib or a pharmaceutically acceptable salt thereof, and the anacetrapib or pharmaceutically acceptable salt thereof is administered orally at a dose of about 40 mg, 60 mg, 80 mg, 100 mg, 120 mg, 140 mg, 160 mg, 180 mg, or 200 mg daily.

In certain embodiments, the CETP inhibitor is evacetrapib or a pharmaceutically acceptable salt thereof, and the evacetrapib or pharmaceutically acceptable salt thereof is administered orally at a dose of about 30 mg, 60 mg, 90 mg, 100 mg, 120 mg, 140 mg, 160 mg, 180 mg, 200 mg, 250 mg, 300 mg, 350 mg, 400 mg, 450 mg, 500 mg, 550 mg, or 600 mg daily.

In certain embodiments, the CETP inhibitor is BAY 60-5521 or a pharmaceutically acceptable salt thereof, and the BAY 60-5521 or pharmaceutically acceptable salt thereof is administered orally at a dose of about 5 mg, 12.5 mg, 25 mg, 30 mg, 40 mg, 50 mg, 60 mg, 70 mg, 80 mg, 90 mg, or 100 mg daily.

In certain embodiments, the antidiabetic agent is metformin or a pharmaceutically acceptable salt thereof, and the metformin or pharmaceutically acceptable salt thereof is administered in amount ranging 100 to 2500 mg daily. In certain embodiments, the antidiabetic agent is metformin or a pharmaceutically acceptable salt thereof, and the metformin or pharmaceutically acceptable salt thereof is administered orally at a dose of about 500 mg, 600 mg, 700 mg, 800 mg, 900 mg, 1000 mg, 1200 mg, 1400 mg, 1600 mg, 1800 mg, 2000 mg, 2200 mg, or 2400 mg daily.

In certain embodiments, the antidiabetic agent is sulfonylurea, and the sulfonylurea is administered in amount ranging 1 to 40 mg daily. In certain embodiments, the sulfonylurea is at a daily dose of about 1 mg, 1.25 mg, 1.5 mg, 2 mg, 2.5 mg, 4 mg, 5 mg, 6 mg, 7 mg, 8 mg, 9 mg, 10 mg, 15 mg, 20 mg, 25 mg, 30 mg, 35 mg, or 40 mg.

In certain embodiments, the antidiabetic agent is a GLP-1 or GLP-1 analogue, and the GLP-1 or GLP-1 analogue is administered in amount ranging 0.1 to 40 mg daily. In certain embodiments, the GLP-1 or GLP-1 analogue is administered at a daily dose of about 0.1 mg, 0.2 mg, 0.4 mg, 0.6 mg, 0.8 mg, 1 mg, 1.2 mg, 1.4 mg, 1.6 mg, 1.8 mg, 2 mg, 2.5 mg, 4 mg, 5 mg, 6 mg, 7 mg, 8 mg, 9 mg, 10 mg, 15 mg, 20 mg, 25 mg, 30 mg, 35 mg, or 40 mg. In certain embodiments, the GLP-1 or GLP-1 analogue is administered ranging 0.5 to 50 mg weekly. In certain embodiments, the GLP-1 or GLP-1 analogue is administered at a weekly dose of about 0.5 mg, 0.6 mg, 0.75 mg, 0.8 mg, 1 mg, 1.2 mg, 1.4 mg, 1.5 mg, 1.6 mg, 1.8 mg, 2 mg, 2.5 mg, 4 mg, 5 mg, 6 mg, 7 mg, 8 mg, 9 mg, 10 mg, 15 mg, 20 mg, 25 mg, 30 mg, 35 mg, 40 mg, 45 mg, or 50 mg.

In certain embodiments, the antidiabetic agent is thiazolidinedione, and the thiazolidinedione is administered in amount ranging 1 to 50 mg daily. In certain embodiments, the thiazolidinedione is at a daily dose of about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg.

In certain embodiments, the antidiabetic agent is alpha-glucosidase blocker, and the alpha-glucosidase blocker is administered in amount ranging 25 to 300 mg daily. In certain embodiments, the alpha-glucosidase blocker is at a daily dose of about 25 mg, 50 mg, 75 mg, 100 mg, 125 mg, 150 mg, 175 mg, 200 mg, 225 mg, 250 mg, 275 mg, or 300 mg.

In certain embodiments, the antidiabetic agent is glinide, and the glinide is administered in amount ranging 0.5 to 360 mg daily. In certain embodiments, the glinide is at a daily dose of about 0.5 mg, 1 mg, 1.25 mg, 1.5 mg, 2 mg, 2.5 mg, 4 mg, 5 mg, 6 mg, 7 mg, 8 mg, 9 mg, 10 mg, 15 mg, 20 mg, 25 mg, 30 mg, 35 mg, 40 mg, 50 mg, 60 mg, 75 mg, 100 mg, 120 mg, 125 mg, 150 mg, 175 mg, 200 mg, 225 mg, 240 mg, 250 mg, 275 mg, 300 mg, or 360 mg.

In certain embodiments, the antidiabetic agent is insulin or insulin analogue, and the insulin or insulin analogue is administered in amount ranging 1 unit to 500 units daily. In certain embodiments, the insulin or insulin analogue is at a daily dose of about 1 unit, 2 units, 3 units, 4 units, 5 units, 6 units, 7 units, 8 units, 9 units, 10 units, 15 units, 20 units, 25 units, 30 units, 40 units, 50 units, 60 units, 70 units, 80 units, 90 units, 100 units, 110 units, 120 units, 130 units, 140 units, 150 units, 160 units, 170 units, 180 units, 190 units, 200 units, 250 units, 300 units, 350 units, 400 units, 450 units, or 500 units.

In certain embodiments, the antidiabetic agent is DPP-IV inhibitor, and the DPP-IV inhibitor is administered in amount ranging 1 to 100 mg daily. In certain embodiments, the DPP-IV inhibitor is at a daily dose of about 1 mg, 1.25 mg, 1.5 mg, 2 mg, 2.5 mg, 4 mg, 5 mg, 6 mg, 7 mg, 8 mg, 9 mg, 10 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg.

### Compositions and Kits

The present invention also provides compositions comprising (a) an effective amount of a CETP inhibitor and an antidiabetic agent inhibitor; and (b) a pharmaceutically acceptable carrier or vehicle. The compositions of the invention are useful for delaying occurrence of new-onset type 2 diabetes, slowing progression of type 2 diabetes, treating type 2 diabetes or slowing progression of a complication of type 2 diabetes.

In some embodiments, the CETP inhibitor is any one of the aforementioned CETP inhibitors. In some embodiments, the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

In some embodiments, the CETP inhibitor of the compositions of the invention is dalcetrapib or a pharmaceutically acceptable salt thereof.

In some embodiments, the antidiabetic agent is metformin or a pharmaceutically acceptable salt thereof.

In some embodiments, the antidiabetic agent is a sulfonylurea. In some embodiments, the sulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analog is exenatide, liraglutide, lixisenatide, albiglutide, dulaglutide, or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir, or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin, or dutogliptin, or a pharmaceutically acceptable salt of any of the foregoing.

In some embodiments, the pharmaceutical acceptable carrier or vehicle is a liquid, such as water and/or oil, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents are useful. In some embodiments, the pharmaceutically acceptable excipients are sterile. Water is a useful excipient, particularly for intravenous compositions of the invention. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The compositions of the invention, if desired, can also comprise minor amounts of wetting or emulsifying agents, or pH buffering agents.

The compositions of the invention can be formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained release formulation; (3) topical administration, for example, as a cream, ointment, or a controlled release patch or spray applied to the skin; (4) intravaginal or intrarectal administration, for example, as a pessary, cream or foam; (5) sublingual administration; (6) ocular administration; (7) transdermal administration; or (8) nasal administration.

Compositions of the invention include those suitable for oral, nasal, topical (including buccal and sublingual), rectal, vaginal and/or parenteral administration. The compositions can be in unit dosage form. The compositions of the invention can be prepared by any methods well known in the art. Generally, out of one hundred percent, the amount of CETP inhibitor or antidiabetic agent present in the compositions of the invention ranges from about 0.1 percent to about ninety-nine percent by weight of the composition, e.g., from about 5 percent to about 70 percent by weight of the composition, or from about 10 percent to about 30 percent by weight of the composition.

In some embodiments, the compositions of the invention comprise a cyclodextrin, cellulose, liposome, micelle-forming, e.g., a bile acid, polymeric carrier, e.g., a polyester or polyanhydride, excipient.

In some embodiments, the compositions of the invention can be made by bringing into association a CETP inhibitor or antidiabetic agent with a carrier and, optionally, one or more accessory ingredients.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like. A CETP inhibitor or antidiabetic agent may also be administered as a bolus, electuary or paste.

Where a composition of the invention is a solid dosage form, (a capsule, tablet, pill, dragee, powder, granule, trouche and the like), the CETP inhibitor or antidiabetic agent can be admixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the compositions of the invention can also comprise one or more buffering agents. The compositions of the invention can be soft- or hard-shelled gelatin capsules comprising fillers or excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the compositions of the invention, such as dragees, capsules, pills and granules, can optionally be scored or prepared with coatings and shells, such as enteric coatings and other coatings known in the art. The compositions of the invention can also be formulated so as to provide slow or controlled release of the CETP inhibitor or antidiabetic agent therein using, for example, hydroxypropylmethyl cellulose in varying proportions to provide the desired release profile, other polymer matrices, liposomes and/or microspheres. The compositions of the invention can be formulated for rapid release, e.g., freeze-dried. The compositions of the invention can be sterilized by, for example, filtration through a bacteria-retaining filter, or by incorporating sterilizing agents in the form of sterile solid compositions which can be dissolved in sterile water, or some other sterile injectable medium immediately before use. The compositions of the invention can also optionally contain one or more opacifying agents or can release the CETP inhibitor or antidiabetic agent only, or preferentially, in a certain portion of the gastrointestinal tract, optionally, in a delayed manner. Examples of embedding excipients that can be used include polymeric substances and waxes. The CETP inhibitor or antidiabetic agent can also be in micro-encapsulated form, if appropriate, with one or more of the above-described excipients.

Liquid dosage forms for oral administration of the CETP inhibitor or antidiabetic agent include pharmaceutically acceptable emulsions, microemulsions, solutions, suspensions, syrups and elixirs. In addition to the CETP inhibitor or antidiabetic agent, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the CETP inhibitor or antidiabetic agent, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions of the invention for rectal or vaginal administration can be formulated as a suppository, which can be prepared by admixing one or both of the CETP inhibitor and antidiabetic agent with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release one or more active compounds.

Compositions of the invention which are suitable for vaginal administration also include pessaries, tampons, creams, gels, pastes, foams or spray compositions containing such carriers as are known in the art to be appropriate.

Compositions of the invention formulated for topical or transdermal administration include powders, sprays, ointments, pastes, creams, lotions, gels, solutions, patches and inhalants. The CETP inhibitor or antidiabetic agent can be mixed under sterile conditions with a pharmaceutically acceptable carrier, and with any preservatives, buffers, or propellants which might be useful.

The ointments, pastes, creams and gels may contain, in addition to CETP inhibitor or antidiabetic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to CETP inhibitor or antidiabetic agent, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a CETP inhibitor or antidiabetic agent to a subject. Such dosage forms can be made by dissolving or dispersing the CETP inhibitor or antidiabetic agent in a suitable medium. Absorption enhancers can also be used to increase the flux of the CETP inhibitor or antidiabetic agent across the skin. The rate of such flux can be controlled by either providing a rate controlling membrane or dispersing the CETP inhibitor or antidiabetic agent in a polymer matrix or gel.

Compositions of the invention suitable for parenteral administration can comprise a pharmaceutically acceptable sterile isotonic aqueous or nonaqueous solution, dispersion, suspension or emulsion, or sterile powder that can be reconstituted into sterile injectable solutions or dispersions prior to use, which may contain sugars, alcohols, antioxidants, buffers, bacteriostats, solutes which render the composition isotonic with the blood of the intended recipient or suspending or thickening agents.

Examples of suitable aqueous and nonaqueous carriers which may be employed in the compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions of the invention can also contain adjuvants such as preservatives, wetting agents, emulsifying agents and dispersing agents. Prevention or retardation of the action of microorganisms upon the compositions of the invention can be achieved by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of an injectable composition of the invention can be brought about by the inclusion of agents which delay absorption such as aluminum monostearate and gelatin.

In some cases, in order to prolong the effect of the CETP inhibitor or antidiabetic agent, it is desirable to slow the absorption of the CETP inhibitor or antidiabetic agent from subcutaneous or intramuscular injection. This can be accomplished by the use of a liquid suspension of crystalline or amorphous material having poor water solubility. The rate of absorption of the CETP inhibitor or antidiabetic agent might then depend upon its rate of dissolution which, in turn, might depend upon its crystal size or crystalline form. Alternatively, delayed absorption of a parenterally administered composition of the invention can be accomplished by dissolving or suspending the CETP inhibitor or antidiabetic agent in an oil vehicle.

Injectable depot compositions of the invention can be made by forming microencapsule matrices of the CETP inhibitor or antidiabetic agent in biodegradable polymers such as polylactide-polyglycolide. Depending on the ratio of CETP inhibitor or antidiabetic agent to polymer, and the nature of the particular polymer employed, the rate of CETP inhibitor or antidiabetic agent release can be controlled. Examples of other biodegradable polymers include poly(orthoesters) and poly(anhydrides). Depot injectable compositions of the invention can also be prepared by entrapping the CETP inhibitor or antidiabetic agent in liposomes or microemulsions that are compatible with body tissue.

In the methods of the invention the CETP inhibitor or antidiabetic agent can be administered per se or as a component of a pharmaceutical composition comprising, for example, 0.1 to 99% (in some embodiments, 10 to 30%) by weight of the composition.

The CETP inhibitor, antidiabetic agent and compositions of the invention can be administered orally, buccally, sublingually, parenterally, intraocularly, parenterally, topically, nasally, via inhalation, intracisternally, subcutaneously, systemically, vaginally or rectally.

For example, the CETP inhibitor, antidiabetic agent and compositions of the invention can be administered in tablets or capsule form, by injection, inhalation, eye lotion, ointment, suppository, etc. administration by injection, infusion or inhalation; topical by lotion or ointment; and rectal by suppositories. In some embodiments, the CETP inhibitor, antidiabetic agent and compositions of the invention are administered orally.

Parenteral administration includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Regardless of the route of administration selected, the CETP inhibitor or antidiabetic agent, which may be used in a suitable hydrated form, and/or the compositions of the invention can be formulated as pharmaceutically acceptable dosage forms using conventional methods known to those of skill in the art.

In some embodiments, a suitable daily dose of a CETP inhibitor or an antidiabetic agent is that amount of the CETP inhibitor or antidiabetic agent which is the lowest dose effective in the compositions or methods of the invention.

If desired, the effective daily dose of the CETP inhibitor or antidiabetic agent can be administered as two, three, four, five, six or more sub-doses administered separately at appropriate intervals throughout the day, optionally, in unit dosage forms, e.g., one administration per day.

The invention also provides kits useful for the methods of the invention. In some embodiments, the kits comprise a CETP inhibitor or an antidiabetic agent and instructions for its use. In some embodiments, each of the CETP inhibitor and antidiabetic agent is present in a separate composition. In some embodiments, the CETP inhibitor and antidiabetic agent are present in the same composition.

The present invention also provides compositions comprising (a) an effective amount of a CETP inhibitor, an ADCY inhibitor and an antidiabetic agent; and (b) a pharmaceutically acceptable carrier or vehicle. The compositions of the invention are useful for delaying occurrence of new-onset type 2 diabetes, slowing progression of type 2 diabetes, treating type 2 diabetes or slowing progression of a complication of type 2 diabetes.

In some embodiments, the CETP inhibitor is any one of the aforementioned CETP inhibitors. In some embodiments, the CETP inhibitor is dalcetrapib, torcetrapib, anacetrapib, evacetrapib, obicetrapib, BMS795311, CP-800,569, DLBS-1449, ATH-03, DRL-17822, JNJ-28545595, JNJ-28614872, BAY 19-4789, BAY 38-1315, or BAY 60-5521, or a pharmaceutically acceptable salt of any of the foregoing.

In some embodiments, the CETP inhibitor of the compositions of the invention is dalcetrapib or a pharmaceutically acceptable salt thereof.

In some embodiments, the ADCY inhibitor is an ADCY1, ADCY2, ADCY3, ADCY4, ADCY5, ADCY6, ADCY7, ADCY8, ADCY9 or ADCY10 inhibitor.

In some embodiments, the ADCY inhibitor is SQ22536 (9-(tetrahydro-2-furanyl)-adenine), 2′,5′-dideoxyadenosine, 9-cyclopentyladenine, 2′,5′-dideoxyadenosine 3′-diphosphate, 2′,5′-dideoxyadenosine 3′-monophosphate, MDL-12330A (cis-N-(2-phenylcyclopentyl)azacyclotridece-1-en-2-amine), compounds such as 7,8-dihydro-5(6H)-quinazolinone derivatives disclosed in JP Patent Application No. 2001-153954 (preferably, 2-amino-7-(4-chlorophenyl)-7,8-dihydro-5 (6H)-quinazolinone, 2-amino-7-(4-methoxyphenyl)-7,8-dihydro-5(6H)-quinazolinone, 2-amino-7-phenyl-7,8-dihydro-5(6H)-quinazolinone, 4.2-amino-7-(2-furanyl)-7,8-dihydro-5(6H)-quinazolinone, and 2-amino-7-(2-thienyl)-7,8-dihydro-5(6H)-quinazolinone), MANT-ATP; MANT-ITP; MANT-GTP; MANT-XTP; MANT-CTP; MANT-UTP; 2′-MANT-3′dATP; 3′-MANT-2′dATP; MANT-ATPTS; MANT-ITPTS; MANT-GTPTS; MANT-UTPTS; ANT-ATP; Cl-ANT-ATP; Cl-ANT-ITP; Br-ANT-ITP; Pr-ANT-ATP; Pr ANT-ITP; AcNH-ANT-ATP; AcNH-ANT-ITP; MANT-AppNHp; MANT-GppNHp; TNP-ATP; TNP-GTP; TNP-CTP; TNP-UTP; Bis-MANT-ATP; Bis-MANT-ITP; Bis-MANT-CTP; Bis-MANT-IDP; Bis-MANT-IMP; Bis-Cl-ANT-ATP; Bis-Cl-ANT-ITP; Bis-Br-ANT-ATP; Bis-Br-ANT-ITP; Bis-Pr-ANT-ATP; Bis-Pr-ANT-ITP; Bis-AcNH-ANT-ATP; Bis-AcNH-ANT-ITP; NKY80; vidarabine; 2′, 5′-dd-3′-ATP; AraAde; PMC6; NB001; BODIPY-FS; 1,9-dd-FS; 6A7DA-FS; Calmidazolium; Tyrphostin A25; 9-Cyclopentyladenine monomethanesulfonate; (E)-2-(1H-Benzo[d]imidazol-2-ylthio)-N′-(5-bromo-2-hydroxybenzylidene)propanehydrazide; SB-268262; LRE1; 2′,5′-Dideoxyadenosine; or 2′,5′-Dideoxyadenosine 3′-triphosphate tetrasodium salt; or a pharmaceutically acceptable salt of any of the foregoing.

In some embodiments, the antidiabetic agent is metformin or a pharmaceutically acceptable salt thereof.

In some embodiments, the antidiabetic agent is a sulfonylurea. In some embodiments, the sulfonylureasulfonylurea is acetohexamide, carbutamide, chlorpropamide, glycyclamide (tolhexamide), metahexamide, tolazamide, tolbutamide, glibenclamide (glyburide), glibornuride, gliclazide, glipizide, gliquidone, glisoxepide, glyclopyramide, or glimepiride, or a pharmaceutically acceptable salt of any of the foregoing.

In some embodiments, the antidiabetic agent is a thiazolidinedione. In some embodiments, the thiazolidinedione is pioglitazone, rosiglitazone, lobeglitazone, ciglitazone, darglitazone, englitazone, netoglitazone, rivoglitazone, troglitazone, or balaglitazone (DRF-2593), or a pharmaceutically acceptable salt of any of the foregoing.

In some embodiments, the antidiabetic agent is a glinide. In some embodiments, the glinide is repaglinide, nateglinide, or mitiglinide, or a pharmaceutically acceptable salt of any of the foregoing.

In some embodiments, the antidiabetic agent is an alpha-glucosidase blocker. In some embodiments, the alpha-glucosidase blocker is acarbose, miglitol, or voglibose, or a pharmaceutically acceptable salt of the foregoing.

In some embodiments, the antidiabetic agent is GLP-1.

In some embodiments, the antidiabetic agent is a GLP-1 analogue. In some embodiments, the GLP-1 analogue is exenatide, liraglutide, lixisenatide, albiglutide, dulaglutide, or semaglutide, or a pharmaceutically acceptable salt of any of the foregoing.

In some embodiments, the antidiabetic agent is insulin.

In some embodiments, the antidiabetic agent is an insulin analogue. In some embodiments, the insulin analogue is glulisine, lispro, aspart, insulin glargine, insulin detemir, or insulin degludec, or a pharmaceutically acceptable salt of any of the foregoing.

In some embodiments, the antidiabetic agent is a DPP-IV inhibitor. In some embodiments, the DPP-IV inhibitor is sitagliptin, vildagliptin, saxagliptin, linagliptin, gemigliptin, anagliptin, teneligliptin, alogliptin, trelagliptin, omarigliptin, evogliptin, gosogliptin, or dutogliptin, or a pharmaceutically acceptable salt of any of the foregoing.

In some embodiments, the pharmaceutical acceptable carrier or vehicle is a liquid, such as water and/or oil, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents are useful. In some embodiments, the pharmaceutically acceptable excipients are sterile. Water is a useful excipient, particularly for intravenous compositions of the invention. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The compositions of the invention, if desired, can also comprise minor amounts of wetting or emulsifying agents, or pH buffering agents.

The compositions of the invention can be formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained release formulation; (3) topical administration, for example, as a cream, ointment, or a controlled release patch or spray applied to the skin; (4) intravaginal or intrarectal administration, for example, as a pessary, cream or foam; (5) sublingual administration; (6) ocular administration; (7) transdermal administration; or (8) nasal administration.

Compositions of the invention include those suitable for oral, nasal, topical (including buccal and sublingual), rectal, vaginal and/or parenteral administration. The compositions can be in unit dosage form. The compositions of the invention can be prepared by any methods well known in the art. Generally, out of one hundred percent, the amount of CETP inhibitor or antidiabetic agent present in the compositions of the invention ranges from about 0.1 percent to about ninety-nine percent by weight of the composition, e.g., from about 5 percent to about 70 percent by weight of the composition, or from about 10 percent to about 30 percent by weight of the composition.

In some embodiments, the compositions of the invention comprise a cyclodextrin, cellulose, liposome, micelle-forming, e.g., a bile acid, polymeric carrier, e.g., a polyester or polyanhydride, excipient.

In some embodiments, the compositions of the invention can be made by bringing into association a CETP inhibitor or antidiabetic agent with a carrier and, optionally, one or more accessory ingredients.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like. A CETP inhibitor or ADCY inhibitor may also be administered as a bolus, electuary or paste.

Where a composition of the invention is a solid dosage form, (a capsule, tablet, pill, dragee, powder, granule, trouche and the like), the CETP inhibitor or ADCY inhibitor can be admixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, some silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the compositions of the invention can also comprise one or more buffering agents. The compositions of the invention can be soft- or hard-shelled gelatin capsules comprising fillers or excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the compositions of the invention, such as dragees, capsules, pills and granules, can optionally be scored or prepared with coatings and shells, such as enteric coatings and other coatings known in the art. The compositions of the invention can also be formulated so as to provide slow or controlled release of the CETP inhibitor or ADCY inhibitor therein using, for example, hydroxypropylmethyl cellulose in varying proportions to provide the desired release profile, other polymer matrices, liposomes and/or microspheres. The compositions of the invention can be formulated for rapid release, e.g., freeze-dried. The compositions of the invention can be sterilized by, for example, filtration through a bacteria-retaining filter, or by incorporating sterilizing agents in the form of sterile solid compositions which can be dissolved in sterile water, or some other sterile injectable medium immediately before use. The compositions of the invention can also optionally contain one or more opacifying agents or can release the CETP inhibitor or ADCY inhibitor only, or preferentially, in a certain portion of the gastrointestinal tract, optionally, in a delayed manner. Examples of embedding excipients that can be used include polymeric substances and waxes. The CETP inhibitor or ADCY inhibitor can also be in micro-encapsulated form, if appropriate, with one or more of the above-described excipients.

Liquid dosage forms for oral administration of the CETP inhibitor or ADCY inhibitor include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions of the invention for rectal or vaginal administration may be presented as a suppository, which may be prepared by admixing one or both of the CETP inhibitor and ADCY inhibitor with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release one or more active compounds.

Compositions of the invention which are suitable for vaginal administration also include pessaries, tampons, creams, gels, pastes, foams or spray compositions containing such carriers as are known in the art to be appropriate.

Compositions of the invention formulated for topical or transdermal administration include powders, sprays, ointments, pastes, creams, lotions, gels, solutions, patches and inhalants. The CETP inhibitor or ADCY inhibitor can be mixed under sterile conditions with a pharmaceutically acceptable carrier, and with any preservatives, buffers, or propellants which might be useful.

The ointments, pastes, creams and gels may contain, in addition to CETP inhibitor or ADCY inhibitor, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to CETP inhibitor or ADCY inhibitor, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a CETP inhibitor or ADCY inhibitor to a subject. Such dosage forms can be made by dissolving or dispersing the CETP inhibitor or ADCY inhibitor in a suitable medium. Absorption enhancers can also be used to increase the flux of the CETP inhibitor or ADCY inhibitor across the skin. The rate of such flux can be controlled by either providing a rate controlling membrane or dispersing the CETP inhibitor or ADCY inhibitor in a polymer matrix or gel.

Compositions of the invention suitable for parenteral administration can comprise a pharmaceutically acceptable sterile isotonic aqueous or nonaqueous solution, dispersion, suspension or emulsion, or sterile powder that can be reconstituted into sterile injectable solutions or dispersions prior to use, which may contain sugars, alcohols, antioxidants, buffers, bacteriostats, solutes which render the composition isotonic with the blood of the intended recipient or suspending or thickening agents.

Examples of suitable aqueous and nonaqueous carriers which may be employed in the compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions of the invention can also contain adjuvants such as preservatives, wetting agents, emulsifying agents and dispersing agents. Prevention or retardation of the action of microorganisms upon the compositions of the invention can be achieved by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of an injectable composition of the invention can be brought about by the inclusion of agents which delay absorption such as aluminum monostearate and gelatin.

In some cases, in order to prolong the effect of the CETP inhibitor or ADCY inhibitor, it is desirable to slow the absorption of the CETP inhibitor or ADCY inhibitor from subcutaneous or intramuscular injection. This can be accomplished by the use of a liquid suspension of crystalline or amorphous material having poor water solubility. The rate of absorption of the CETP inhibitor or ADCY inhibitor might then depend upon its rate of dissolution which, in turn, might depend upon its crystal size or crystalline form. Alternatively, delayed absorption of a parenterally administered composition of the invention can be accomplished by dissolving or suspending the CETP inhibitor or ADCY inhibitor in an oil vehicle.

Injectable depot compositions of the invention can be made by forming microencapsule matrices of the CETP inhibitor or ADCY inhibitor in biodegradable polymers such as polylactide-polyglycolide. Depending on the ratio of CETP inhibitor or ADCY inhibitor to polymer, and the nature of the particular polymer employed, the rate of CETP inhibitor or ADCY inhibitor release can be controlled. Examples of other biodegradable polymers include poly(orthoesters) and poly(anhydrides). Depot injectable compositions of the invention can also be prepared by entrapping the CETP inhibitor or ADCY inhibitor in liposomes or microemulsions that are compatible with body tissue.

In the methods of the invention the CETP inhibitor or ADCY inhibitor can be administered per se or as a component of a pharmaceutical composition comprising, for example, 0.1 to 99% (in some embodiments, 10 to 30%) by weight of the composition.

The CETP inhibitor, ADCY inhibitor and compositions of the invention can be administered orally, buccally, sublingually, parenterally, intraocularly, parenterally, topically, nasally, via inhalation, intracisternally, subcutaneously, systemically, vaginally or rectally.

For example, the CETP inhibitor, ADCY inhibitor and compositions of the invention can be administered in tablets or capsule form, by injection, inhalation, eye lotion, ointment, suppository, etc. administration by injection, infusion or inhalation; topical by lotion or ointment; and rectal by suppositories. In some embodiments, the CETP inhibitor, ADCY inhibitor and compositions of the invention are administered orally.

Parenteral administration includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Regardless of the route of administration selected, the CETP inhibitor or ADCY inhibitor, which may be used in a suitable hydrated form, and/or the pharmaceutical compositions of the present invention, can be formulated into pharmaceutically acceptable dosage forms by conventional methods known to those of skill in the art.

In some embodiments, a suitable daily dose of a CETP inhibitor or an ADCY inhibitor is that amount of the CETP inhibitor or ADCY inhibitor which is the lowest dose effective in the compositions or methods of the invention.

If desired, the effective daily dose of the active compound can be administered as two, three, four, five, six or more sub-doses administered separately at appropriate intervals throughout the day, optionally, in unit dosage forms, e.g., one administration per day.

The invention also provides kits useful for the methods of the invention. In some embodiments, the kits comprise a CETP inhibitor or an ADCY inhibitor and instructions for its use. In some embodiments, each of the CETP inhibitor and ADCY inhibitor is present in a separate composition. In some embodiments, the CETP inhibitor and ADCY inhibitor are present in the same composition.

### EXAMPLES

### Example 1: Effects of ADCY9 Genotypes on Change in Glycemia

The effect of ADCY9 rs1967309 genotypes on patient HbA1C and glucose levels was retrospectively assessed in patients enrolled in the dal-OUTCOMES trial.

Inclusion Criteria: Patients 45 years of age or older who provided written informed consent were eligible to participate if they had been hospitalized for an acute coronary syndrome characterized by elevated cardiac biomarkers, with symptoms of acute myocardial ischemia, ischemic electrocardiographic abnormalities that were new or presumed to be new, or loss of viable myocardium on imaging. Patients without elevated cardiac biomarkers were eligible to participate if symptoms of acute myocardial ischemia were accompanied by electrocardiographic changes that were new or presumed to be new and by additional evidence of obstructive coronary disease. Patients who had a myocardial infarction associated with percutaneous coronary intervention were also eligible. All patients had to be following individualized, evidence-based programs for lowering their LDL cholesterol levels by means of statin therapy (if they did not have unacceptable side effects) and diet, with a target LDL cholesterol level of 100 mg per deciliter (2.6 mmol per liter) or lower and preferably 70 mg per deciliter (1.8 mmol per liter) or lower. However, no specific statin agent or dose was specified, and patients were not excluded if their LDL cholesterol level remained above 100 mg per deciliter. There were no exclusions on the basis of patients' HDL cholesterol level. Exclusion Criteria: Patients with serum triglyceride levels of 400 mg per deciliter (4.5 mmol per liter) or higher were excluded; Females who are pregnant or breast-feeding; Women of childbearing potential (women who are not surgically sterile or postmenopausal defined as amenorrhea for >12 months) who are not using a highly effective contraceptive method (failure rate less than 1% per year) such as implants, injectables, combined oral contraceptives. The patients began a single-blind placebo-based run-in period of approximately 4 to 6 weeks to allow for patients to stabilize and for completion of any planned revascularization procedures. At the end of the run-in period, patients in stable condition were randomized in a 1:1 ratio to 600 mg of dalcetrapib or placebo on top of evidence-based medical care for acute cardiovascular syndrome (“ACS”). The descriptive statistics and analyses were performed using SAS 9.4 software.

Cox proportional hazards regression of single nucleotide polymorphism (“SNP”) rs1967309 was conducted for association with cardiovascular events in each treatment arm and in patients with a diagnosis of diabetic at baseline in the dal-OUTCOMES trial and in non-diabetic patients separately without controlling for any covariate, as shown in Table 4. Cox proportional hazards regression of SNP rs1967309 was conducted for association with cardiovascular events in each treatment arm and in diabetic and non-diabetic patients separately controlling for age and sex, as shown in Table 5. At a significance level of 5%, SNP rs1967309 was predictive of cardiovascular events (time to first occurrence of death from coronary heart disease, nonfatal myocardial infarction, ischemic stroke, unstable angina, cardiac arrest with resuscitation, or unscheduled coronary revascularization) in the dalcetrapib arm for diabetic and non-diabetic patients with and without controlling for the covariates (see Table 4 and Table 5).

TABLE 4

| | | | | Global | | \hat{β} | \hat{β} | | LCL | UCL |
|---|---|---|---|---|---|---|---|---|---|---|
| RNDGRP | Patients | Event | Censored | p-value | \hat{β}_{genetic} | StdErr | p-value | HR | HR | HR |
| Dalcetrapib | Non- | 255 | 1954 | 1.73E−06 | −0.4462 | 0.0955 | 2.98E−06 | 0.6400 | 0.5308 | 0.7718 |
| | diabetic | | | | | | | | | |
| | Diabetic | 134 | 494 | 0.0009 | −0.4159 | 0.1278 | 0.0011 | 0.6597 | 0.5136 | 0.8475 |
| Placebo | Non- | 269 | 1993 | 0.1701 | −0.1224 | 0.0896 | 0.1719 | 0.8848 | 0.7423 | 1.0547 |
| | diabetic | | | | | | | | | |
| | Diabetic | 128 | 501 | 0.7908 | −0.0332 | 0.1256 | 0.7916 | 0.9674 | 0.7563 | 1.2373 |

TABLE 5

| | | | | Global | | \hat{β} | \hat{β} | | LCL | UCL |
|---|---|---|---|---|---|---|---|---|---|---|
| RNDGRP | Patents | Event | Censored | p-value | \hat{β}_{genetic} | StdErr | p-value | HR | HR | HR |
| Dalcetrapib | Non- | 255 | 1954 | 8.44E−06 | −0.4516 | 0.0956 | 2.32E−06 | 0.6366 | 0.5279 | 0.7678 |
| | diabetic | | | | | | | | | |
| | Diabetic | 134 | 494 | 0.0011 | −0.4108 | 0.1281 | 0.0013 | 0.6631 | 0.5159 | 0.8523 |
| Placebo | Non- | 269 | 1993 | 0.2269 | −0.1232 | 0.0897 | 0.1696 | 0.8841 | 0.7416 | 1.0540 |
| | diabetic | | | | | | | | | |
| | Diabetic | 128 | 501 | 0.8464 | −0.0345 | 0.1254 | 0.7834 | 0.9661 | 0.7556 | 1.2353 |

Cox proportional hazards regression of diabetes was assessed for association with cardiovascular events in genotypes rs1967309/AA, rs1967309/AG and rs1967309/GG and in each treatment arm separately without controlling for any covariate, as shown in Table 3. Cox proportional hazards regression of diabetes was assessed for association with cardiovascular events in genotypes rs1967309/AA, rs1967309/AG and rs1967309/GG and in each treatment arm separately controlling for age and sex, as shown in Table 4. Diabetes was predictive of cardiovascular events for each genotype of the SNP rs1967309 with and without controlling for the covariates in both arms, except for the AA genotype in the group dalcetrapib (see Table 6 and Table 7), demonstrating a cardiovascular protective effect of dalcetrapib in AA patients with diabetes.

TABLE 6

| | Treatment | | | Global | | \hat{β} | \hat{β} | | | |
|---|---|---|---|---|---|---|---|---|---|---|
| rs1967309 | arm | Event | Censored | p-value | \hat{β}_{diabetes} | StdErr | p-value | HR | LCL HR | UCL HR |
| GG | Dalcetrapib | 176 | 801 | 0.0006 | 0.5750 | 0.1611 | 0.0004 | 1.7772 | 1.2959 | 2.4371 |
| | Placebo | 146 | 860 | 0.0169 | 0.4484 | 0.1816 | 0.0135 | 1.5659 | 1.0969 | 2.2353 |
| AG | Dalcetrapib | 176 | 1200 | 1.94E−07 | 0.8501 | 0.1558 | 4.87E−08 | 2.3398 | 1.7241 | 3.1754 |
| | Placebo | 192 | 1218 | 1.81E−05 | 0.6836 | 0.1526 | 7.49E−06 | 1.9810 | 1.4689 | 2.6717 |
| AA | Dalcetrapib | 37 | 447 | 0.3302 | 0.3597 | 0.3597 | 0.3172 | 1.4330 | 0.7081 | 2.8999 |
| | Placebo | 59 | 416 | 0.0515 | 0.5546 | 0.2751 | 0.0438 | 1.7413 | 1.0156 | 2.9857 |

TABLE 7

| | Treatment | | | Global | | \hat{β} | \hat{β} | | LCL | UCL |
|---|---|---|---|---|---|---|---|---|---|---|
| rs1967309 | arm | Event | Censored | p-value | \hat{β}_{diabetes} | StdErr | p-value | HR | HR | HR |
| GG | Dalcetrapib | 176 | 801 | 0.0003 | 0.5255 | 0.1634 | 0.0013 | 1.6913 | 1.2278 | 2.3298 |
| | Placebo | 146 | 860 | 0.0731 | 0.4219 | 0.1836 | 0.0215 | 1.5249 | 1.0641 | 2.1853 |
| AG | Dalcetrapib | 176 | 1200 | 3.75E−07 | 0.7983 | 0.1574 | 3.93E−07 | 2.2217 | 1.6320 | 3.0244 |
| | Placebo | 192 | 1218 | 0.0003 | 0.6732 | 0.1533 | 1.13E−05 | 1.9605 | 1.4516 | 2.6477 |
| AA | Dalcetrapib | 37 | 447 | 0.0282 | 0.5218 | 0.3642 | 0.1520 | 1.6850 | 0.8252 | 3.4406 |
| | Placebo | 59 | 416 | 0.1304 | 0.5105 | 0.2794 | 0.0677 | 1.6661 | 0.9635 | 2.8810 |

Descriptive statistics of reported and change from baseline of glucose and HbA1C according to the genotype of the SNP rs1967309 were analyzed for each treatment arm and for diabetic and non-diabetic patients separately with non-parametric statistics. There was a systematically lower whole-blood level of HbA1c with dalcetrapib treatment compared to placebo in non-diabetic patients irrespective of rs1967309 genotype, and this was confirmed with greater reductions in HbA1c in with measures of change from baseline.

Generalized linear model (“GLM”) results of SNP rs1967309 for fasting plasma glucose at month 1 and whole-blood HbA1C at month 6 were assessed in each treatment arm adjusted for the baseline measures, as shown in Table 5. GLM results of SNP rs1967309 for fasting plasma glucose at month 1 and whole-blood HbA1C at month 6 were assessed in each treatment arm, as shown in Table 6. For the outcome HbA1c at 6 months using GLM, the interaction between SNP rs1967309 and diabetes was significant with and without the additional adjustment for age and sex (see Table 8 and Table 9 respectively).

TABLE 8

| | | | | Std of \hat{β} | p-value | |
|---|---|---|---|---|---|---|
| | Treatment | | \hat{β} gene × | gene × | \hat{β} gene × | R² |
| Outcome | arm | N | diabetes | diabetes | diabetes | model |
| Glucose | Dalcetrapib | 2769 | 0.0025 | 0.0083 | 0.7676 | 0.6186 |
| | Placebo | 2805 | 0.0031 | 0.0085 | 0.7112 | 0.5947 |
| HbA1c | Dalcetrapib | 2702 | −0.0093 | 0.0044 | 0.0326 | 0.7401 |
| | Placebo | 2770 | −0.0094 | 0.0043 | 0.0266 | 0.7174 |

TABLE 9

| | | | | Std of \hat{β} | p-value | |
|---|---|---|---|---|---|---|
| | Treatment | | \hat{β} gene × | gene × | \hat{β} gene × | R² |
| Outcome | arm | N | diabetes | diabetes | diabetes | model |
| Glucose | Dalcetrapib | 2769 | 0.0024 | 0.0083 | 0.7745 | 0.6187 |
| | Placebo | 2805 | 0.0031 | 0.0085 | 0.7111 | 0.5947 |
| HbA1c | Dalcetrapib | 2702 | −0.0095 | 0.0043 | 0.0285 | 0.7407 |
| | Placebo | 2770 | −0.0095 | 0.0043 | 0.0260 | 0.7182 |

For the outcome HbA1c using the repeated measures with mixed regression models adjusted for baseline values and visit, the dalcetrapib treatment arm was significant for all the genotypes of the SNP rs1967309 in diabetic and non-diabetic patients with and without controlling for the additional covariates age and sex, except in the AG diabetic patients (see Table 10 and Table 11). Table 10 shows repeated measures analysis results, using mixed model regression, of dalcetrapib treatment arms for fasting plasma glucose (at month 1, 3, 6, 12, 20, 28) and whole-blood HbA1C (at month 6, 12, 24) for each genotype of SNP rs1967309 and in diabetic and non-diabetic patients separately controlling for baseline measures and visit. Table 8 shows repeated measures results, using mixed model regression, of treatment arms for fasting plasma glucose (at month 1, 3, 6, 12, 20, 28) and whole-blood HbA1C (at month 6, 12, 24) for each genotype of SNP rs1967309 and in diabetic and non-diabetic patients separately controlling for baseline measures, age, sex, and visit.

TABLE 10

| | | | type3 |
|---|---|---|---|
| Outcome | rs1967309 | Patients | p-value |
| Glucose | GG | Non-diabetic | 0.5569 |
| | | Diabetic | 0.4393 |
| | AG | Non-diabetic | 0.1377 |
| | | Diabetic | 0.7956 |
| | AA | Non-diabetic | 0.3982 |
| | | Diabetic | 0.0246 |
| HbA1c | GG | Non-diabetic | 5.47E−10 |
| | | Diabetic | 0.0262 |
| | AG | Non-diabetic | 1.07E−11 |
| | | Diabetic | 0.3509 |
| | AA | Non-diabetic | 4.88E−05 |
| | | Diabetic | 0.0258 |

### Example 2: Effect of Dalcetrapib on HbA1c

TABLE 11

| | | | type3 |
|---|---|---|---|
| Outcome | rs1967309 | Patients | p-value |
| Glucose | GG | Non-diabetic | 0.5643 |
| | | Diabetic | 0.4422 |
| | AG | Non-diabetic | 0.1465 |
| | | Diabetic | 0.8134 |
| | AA | Non-diabetic | 0.3868 |
| | | Diabetic | 0.0315 |
| HbA1c | GG | Non-diabetic | 5.64E−10 |
| | | Diabetic | 0.0257 |
| | AG | Non-diabetic | 1.16E−11 |
| | | Diabetic | 0.3737 |
| | AA | Non-diabetic | 4.41E−05 |
| | | Diabetic | 0.0287 |

The effect of dalcetrapib on whole-blood HbA1c levels irrespective of genotype and the impact of dalcetrapib on risk of new onset diabetes were retrospectively assessed in patients of the dal-OUTCOMES trial. The descriptive statistics and analyses were performed using SAS 9.4 software.

Diabetes at baseline was defined based on at least one of the following patient criteria: (1) diagnosis of diabetics at baseline in the dal-OUTCOMES trial; (2) whole-blood HbA1c level>=6.5% at baseline; (3) fasting glucose level>=7.0 mmol/L at baseline; and (4) use of diabetes medication at or before baseline.

New onset diabetes was defined in non-diabetic patients at baseline based on at least one of the following patient criteria: (1) adverse event (AE) preferred terms “type 2 diabetes mellitus” OR “diabetes mellitus” from the AE file that occurred after randomization; (2) use of diabetes medication that was initiated after randomization; (2) at least one whole-blood HbA1c measurement of >=6.5% after randomization; and (4) at least one fasting glucose measurement of >=126 mg/dl or >=7.0 mmol/L after randomization.

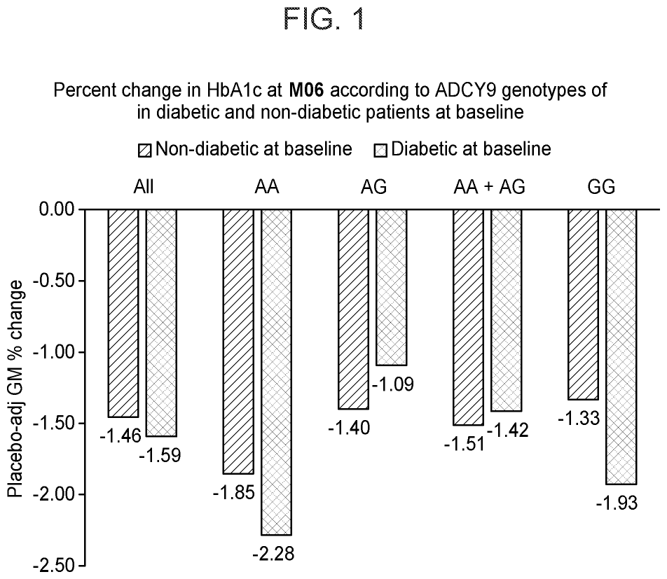

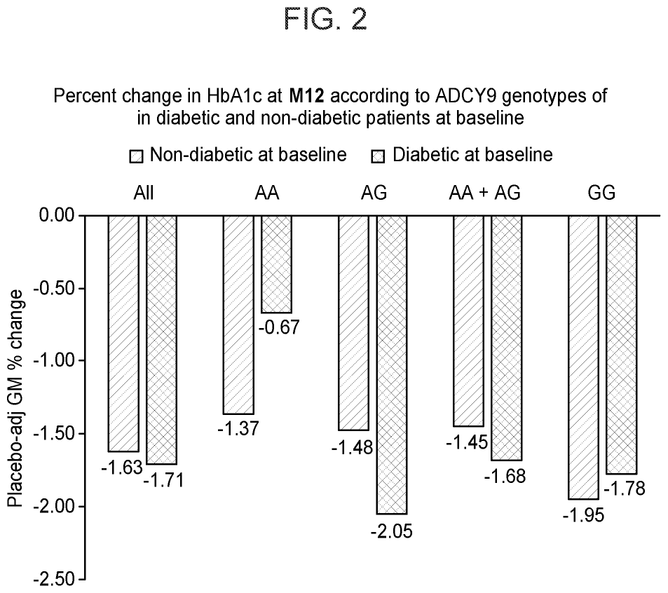

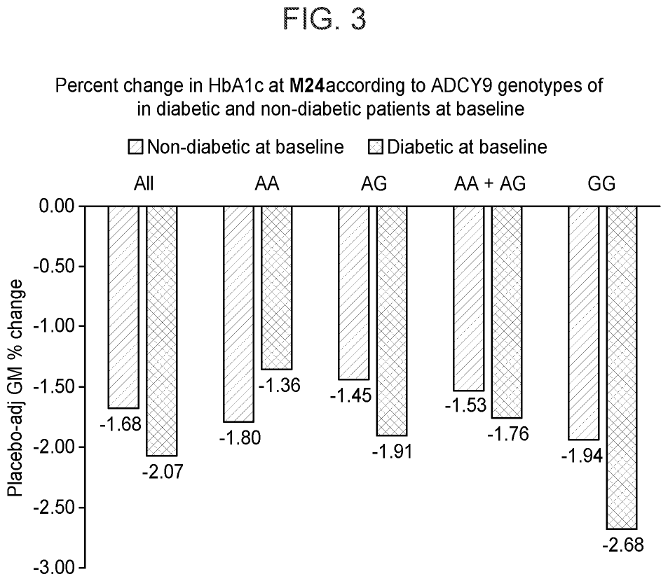

GLM results of the dalcetrapib treatment arm for whole-blood HbA1c at 6,12, and 24 months (“M06”, “M12” and “M24”, respectively) were obtained. At a significance level of the dalcetrapib treatment arm with adjustment for base line value was associated with decrease in whole-blood HbA1c levels at M06 (shown in FIG. 1), M12 (shown in FIG. 2), and M24 (shown in FIG. 3) for all patients combined and for each genotype of the SNP rs1967309 with and without the additional adjustment for the covariates age and sex. The mean ln(HbA1c) in the dalcetrapib treatment arm was lower than in the placebo arm (see Table 12 for results adjusting for baseline HbA1c value).

TABLE 12

| Group | Ln (Outcome) | N | \hat{β} dalcetrapib | Std of \hat{β} | \hat{β} p-value | R² model |
|---|---|---|---|---|---|---|
| All | HBA1C_M06 | 5490 | −0.0143 | 0.0018 | 1.47E−15 | 0.7185 |
| All | HBA1C_M12 | 5277 | −0.0160 | 0.0022 | 4.30E−13 | 0.6318 |
| All | HBA1C_M24 | 4915 | −0.0176 | 0.0025 | 1.51E−12 | 0.5835 |
| genotype = AA | HBA1C_M06 | 923 | −0.0196 | 0.0041 | 1.88E−06 | 0.7075 |
| genotype = AG | HBA1C_M06 | 2660 | −0.0123 | 0.0026 | 2.75E−06 | 0.7149 |
| genotype = GG | HBA1C_M06 | 1899 | −0.0145 | 0.0030 | 1.87E−06 | 0.7297 |
| genotype = AA | HBA1C_M12 | 895 | −0.0116 | 0.0052 | 0.0256 | 0.6176 |
| genotype = AG | HBA1C_M12 | 2564 | −0.0156 | 0.0033 | 2.03E−06 | 0.6130 |
| genotype = GG | HBA1C_M12 | 1810 | −0.0186 | 0.0036 | 3.04E−07 | 0.6654 |
| genotype = AA | HBA1C_M24 | 822 | −0.0171 | 0.0059 | 0.0040 | 0.5724 |
| genotype = AG | HBA1C_M24 | 2388 | −0.0153 | 0.0035 | 1.68E−05 | 0.5788 |
| genotype = GG | HBA1C_M24 | 1697 | −0.0210 | 0.0043 | 1.31E−06 | 0.5946 |
| genotype_group = AA + AG | HBA1C_M06 | 3583 | −0.0141 | 0.0022 | 2.00E−10 | 0.7123 |
| genotype_group = GG | HBA1C_M06 | 1899 | −0.0145 | 0.0030 | 1.87E−06 | 0.7297 |
| genotype_group = AA + AG | HBA1C_M12 | 3459 | −0.0146 | 0.0028 | 1.62E−07 | 0.6140 |
| genotype_group = GG | HBA1C_M12 | 1810 | −0.0186 | 0.0036 | 3.04E−07 | 0.6654 |
| genotype_group = AA + AG | HBA1C_M24 | 3210 | −0.0157 | 0.0030 | 2.49E−07 | 0.5773 |
| genotype_group = GG | HBA1C_M24 | 1697 | −0.0210 | 0.0043 | 1.31E−06 | 0.5946 |
| All | HBA1C_M06 | 5490 | −0.0137 | 0.0018 | 7.71E−15 | 0.7281 |
| All | HBA1C_M12 | 5277 | −0.0152 | 0.0022 | 2.18E−12 | 0.6452 |
| All | HBA1C_M24 | 4915 | −0.0170 | 0.0024 | 3.40E−12 | 0.6017 |
| genotype = AA | HBA1C_M06 | 923 | −0.0192 | 0.0040 | 2.40E−06 | 0.7137 |
| genotype = AG | HBA1C_M06 | 2660 | −0.0113 | 0.0026 | 1.28E−05 | 0.7247 |
| genotype = GG | HBA1C_M06 | 1899 | −0.0145 | 0.0030 | 1.16E−06 | 0.7414 |
| genotype = AA | HBA1C_M12 | 895 | −0.0112 | 0.0051 | 0.0299 | 0.6280 |
| genotype = AG | HBA1C_M12 | 2564 | −0.0143 | 0.0032 | 9.91E−06 | 0.6266 |
| genotype = GG | HBA1C_M12 | 1810 | −0.0185 | 0.0035 | 1.83E−07 | 0.6807 |
| genotype = AA | HBA1C_M24 | 822 | −0.0172 | 0.0059 | 0.0034 | 0.5860 |
| genotype = AG | HBA1C_M24 | 2388 | −0.0139 | 0.0035 | 0.0001 | 0.5966 |
| genotype = GG | HBA1C_M24 | 1697 | −0.0214 | 0.0042 | 4.03E−07 | 0.6168 |
| genotype_group = AA + AG | HBA1C_M06 | 3583 | −0.0133 | 0.0022 | 1.23E−09 | 0.7208 |
| genotype_group = GG | HBA1C_M06 | 1899 | −0.0145 | 0.0030 | 1.16E−06 | 0.7414 |
| genotype_group = AA + AG | HBA1C_M12 | 3459 | −0.0135 | 0.0027 | 7.97E−07 | 0.6266 |
| genotype_group = GG | HBA1C_M12 | 1810 | −0.0185 | 0.0035 | 1.83E−07 | 0.6807 |
| genotype_group = AA + AG | HBA1C_M24 | 3210 | −0.0147 | 0.0030 | 9.00E−07 | 0.5936 |
| genotype_group = GG | HBA1C_M24 | 1697 | −0.0214 | 0.0042 | 4.03E−07 | 0.6168 |

Results were similar for the outcome HbA1c using the repeated measures with mixed regression models, the dalcetrapib treatment arm was a significant predictor of reduced HbA1c for all patients combined and for each genotype of the SNP rs1967309 with and without adjusting for the covariates.

Cox proportional hazards regression of dalcetrapib treatment arm for association with new onset diabetes was assessed. There were 598 (14%) new onset diabetes cases in the 4173 non-diabetics at baseline. New onset diabetes was not found to be significantly associated at P<0.05 with dalcetrapib treatment arm in non-diabetic patients at baseline for all patients combined and for each genotype of the SNP rs1967309 with and without adjusting for the covariates. However, a trend for a protective effect of dalcetrapib on new onset diabetes was observed. There was a significant association with treatment arm in patients with diabetes at baseline and with AA genotype with and without adjustment for covariates; but the number of patients with events was small (n=27).

### Example 3: Effect of Dalcetrapib in Uncontrolled Diabetics

The effect of dalcetrapib in uncontrolled diabetics defined once as HbA1c>7% at baseline or once as >7.5% at baseline was retrospectively assessed in patients of the dal-OUTCOMES trial. The descriptive statistics and analyses were performed using SAS 9.4 software.

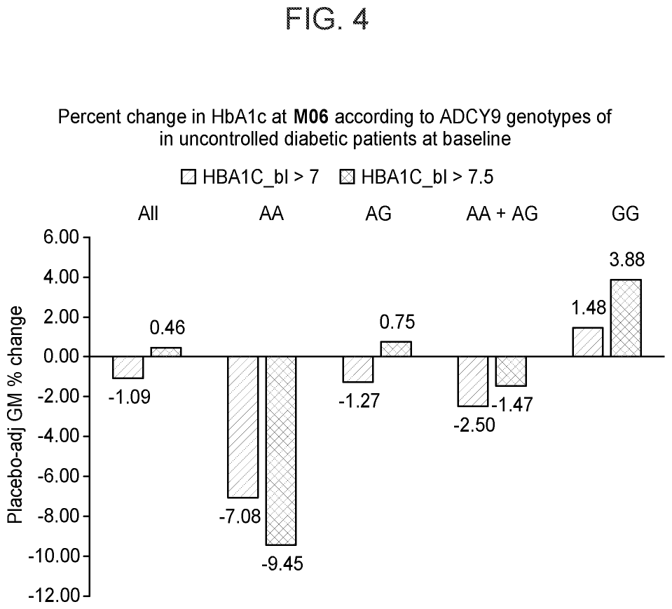

Two populations of uncontrolled diabetics at baseline were defined: patients having a whole-blood HbA1c level of >7 (n=437) and patients having a whole-blood HbA1c level of >7.5 (n=280). At a significance level of 5%, the treatment arm (dalcetrapib versus placebo) was associated with a decrease in whole-blood HbA1c at M06 for uncontrolled diabetic patients having a whole-blood HbA1c level of >7 at baseline and genotype rs1967309/AA without adjustment for the covariates; this association was also shown for uncontrolled diabetic patients having a whole-blood HbA1c level of >7.5 at baseline with genotype rs1967309/AA with and without adjustment for the covariates. The mean ln(HbA1c) in the dalcetrapib treatment arm was lower than in the placebo arm. See FIG. 4. This result was confirmed by repeated measures analysis using mixed regression models for the natural logarithm of HbA1C at 6, 12, and 24 months in uncontrolled diabetic patients.

1.-28. (canceled)

29. A method for treating type 2 diabetes, comprising administering an effective amount of a CETP inhibitor to a subject in need thereof and known to have genotype rs1967309/AA or rs1967309/AG.

30. The method of claim 29, wherein the CETP inhibitor is: dalcetrapib; torcetrapib; anacetrapib; evacetrapib; obicetrapib; BMS795311; CP-800,569; DLBS-1449; ATH-03; DRL-17822; JNJ-28545595; JNJ-28614872; BAY 19-4789; BAY 38-1315; BAY 60-5521; S-[2-(1-isopentylcyclohexanecarbonylamino)phenyl]2,2-dimethylthiopropionate; S-[2-(1-isopentylcyclohexanecarbonylamino)phenyl]2-acetylamino-3-phenylthiopropionate; S-[2-(1-isopentylcyclohexanecarbonylamino)phenyl]3-pyridinethiocarboxylate; S-[2-(1-isopentylcyclohexanecarbonylamino)phenyl]chlorothioacetate; S-[2-(1-isopentylcyclohexanecarbonylamino)phenyl]methoxythioacetate; S-[2-(1-isopentylcyclohexanecarbonylamino)phenyl]thiopropionate; S-[2-(1-isopentylcyclohexanecarbonylamino)phenyl]phenoxy-thioacetate; S-[2-(1-isopentylcyclohexanecarbonylamino)phenyl]2-methylthiopropionate; S-[2-(1-isopentylcyclohexanecarbonylamino)phenyl]4-chlorophenoxythioacetate; S-[2-(1-isopentylcyclohexanecarbonylamino)phenyl]cyclopropanethiocarboxylate; S-[2-(1-isopentylcyclohexanecarbonylamino)phenyl]2-acetylamino-4-carbamoylthiobutyrate; S-[2-(1-isopentylcyclohexanecarbonylamino)phenyl]2-hydroxy-2-methylthiopropionate; S-[2-(1-isopentylcyclopentanecarbonylamino)phenyl]2,2-dimethylthiopropionate; S-[2-(1-isopentylcyclopentanecarbonylamino)phenyl]thioacetate; S-[4,5-dichloro-2-(1-isopentylcyclohexanecarbonylamino)-phenyl]2,2-dimethylthiopropionate; S-[4,5-dichloro-2-(1-isopentylcyclopentanecarbonylamino)-phenyl]2,2-dimethylthiopropionate; S-[2-(1-isopentylcyclohexanecarbonylamino)-4-trifluoromethylphenyl]2,2-dimethylthiopropionate; O-methyl S-[2-(1-isopentylcyclohexanecarbonylaminophenyl monothiocarbonate; S-[2-(1-methylcyclohexanecarbonylamino)phenyl]S-phenyldithiocarbonate; S-[2-(1-isopentylcyclohexanecarbonylamino)phenyl]N-phenylthiocarbamate; S-[2-(pivaloylamino)-4-trifluoromethylphenyl]2,2-dimethylthiopropionate; S-[4,5-dichloro-2-(1-cyclopropylcyclohexanecarbonylamino)phenyl]2,2-dimethylthiopropionate; S-[4,5-dichloro-2-(2-cyclohexylpropionylamino)phenyl]2,2-dimethylthiopropionate; S-[4,5-dichloro-2-(1-pentylcyclohexanecarbonylamino)-phenyl]2,2-dimethylthiopropionate; S-[4,5-dichloro-2-(1-cyclopropylmethylcyclohexanecarbonylamino)phenyl]2,2-dimethylthiopropionate; S-[4,5-dichloro-2-(1-cyclohexylmethylcyclohexanecarbonylamino)phenyl]2,2-dimethylthiopropionate; S-[4,5-dichloro-2-(1-isopropylcyclohexanecarbonylamino)-phenyl]2,2-dimethylthiopropionate; S-[4,5-dichloro-2-(1-isopentylcycloheptanecarbonylamino)-phenyl]2,2-dimethylthiopropionate; S-[4,5-dichloro-2-(1-isopentylcyclobutanecarbonylamino)-phenyl]2,2-dimethylthiopropionate; S-[2-(1-isopentylcyclohexanecarbonylamino)-4-nitrophenyl]2,2-dimethylthiopropionate; S-[4-cyano-2-(1-isopentylcyclohexanecarbonylamino)phenyl]2,2-dimethylthiopropionate; S-[4-chloro-2-(1-isopentylcyclohexanecarbonylamino)phenyl]2,2-dimethylthiopropionate; S-[5-chloro-2-(1-isopentylcyclohexanecarbonylamino)phenyl]2,2-dimethylthiopropionate; S-[4-fluoro-2-(1-isopentylcyclohexanecarbonylamino)phenyl]2,2-dimethylthiopropionate; S-[4,5-difluoro-2-(1-isopentylcyclohexanecarbonylamino)-phenyl]2,2-dimethylthiopropionate; S-[5-fluoro-2-(1-isopentylcyclohexanecarbonylamino)phenyl]2,2-dimethylthiopropionate; bis-[4,5-dichloro-2-(1-isopentylcyclohexanecarbonylamino)-phenyl]disulfide; 2-tetrahydrofurylmethyl 2-(1-isopentylcyclohexanecarbonylamino)phenyl disulfide; N-(2-mercaptophenyl)-1-ethylcyclohexanecarboxamide; N-(2-mercaptophenyl)-1-propylcyclohexanecarboxamide; N-(2-mercaptophenyl)-1-butylcyclohexanecarboxamide; N-(2-mercaptophenyl)-1-isobutylcyclohexanecarboxamide; S-[2-(1-isopentylcyclohexanecarbonylamino)phenyl]cyclohexanethiocarboxylate; S-[2-(1-isopentylcyclohexanecarbonylamino)phenyl]thiobenzoate; S-[2-(1-isopentylcyclohexanecarbonylamino)phenyl]5-carboxythiopentanoate; S-[2-(1-isopentylcyclohexanecarbonylamino)-4-methylphenyl]thioacetate; bis-[2-[1-(2-ethylbutyl)cyclohexanecarbonylamino]phenyl]disulfide; N-(2-mercaptophenyl)-1-(2-ethylbutyl)cyclohexanecarboxamide; S-[2-[1-(2-ethylbutyl)cyclohexanecarbonylamino]phenyl]2-methylthiopropionate; S-[2-(1-isobutylcyclohexanecarbonylamino)phenyl]2-methylthiopropionate; S-[2-[1-(2-ethylbutyl)cyclohexanecarbonylamino]phenyl]1-acetylpiperidine-4-thiocarboxylate; S-[2-[1-(2-ethylbutyl)cyclohexanecarbonylamino]phenyl]thioacetate; S-[2-[1-(2-ethylbutyl)cyclohexanecarbonylamino]phenyl]2,2-dimethylthiopropionate; S-[2-[1-(2-ethylbutyl)cyclohexanecarbonylamino]phenyl]methoxythioacetate; S-[2-[1-(2-ethylbutyl)cyclohexanecarbonylamino]phenyl]2-hydroxy-2-methylthiopropionate; S-[2-[1-(2-ethylbutyl)cyclohexanecarbonylamino]phenyl]4-chlorophenoxythioacetate; S-[2-(1-isobutylcyclohexanecarbonylamino)phenyl]4-chlorophenoxythioacetate; or S-[2-(1-isobutylcyclohexanecarbonylamino)phenyl]-1-acetyl-piperidine-4-thiocarboxylate; or a pharmaceutically acceptable salt of any of the foregoing.

31. The method of claim 29, wherein the CETP inhibitor is administered to the subject in an amount ranging from 100 mg to 2400 mg per day.

32. The method of claim 31, wherein the CETP inhibitor is administered to the subject in an amount ranging from 100 mg to 1800 mg per day.

33. The method of claim 32, wherein the CETP inhibitor is administered to the subject in an amount ranging from 300 mg to 900 mg per day.

34. The method of claim 33, wherein the CETP is administered to the subject in an amount of 600 mg per day.

35. The method of claim 29, wherein the method further comprises administering to the subject an effective amount of an antidiabetic agent.

36. The method of claim 35, wherein the antidiabetic agent is metformin, a sulfonylurea, a thiazolidinedione, a glinide, an alpha-glucosidase blocker, GLP-1, a GLP-1 analogue, insulin, an insulin analogue, or a DPP-IV inhibitor, or a pharmaceutically acceptable salt thereof.

37. The method of claim 29, wherein the subject undergoes treatment with an antidiabetic agent.

38. The method of claim 37, wherein the antidiabetic agent is metformin, a sulfonylurea, a thiazolidinedione, a glinide, an alpha-glucosidase blocker, GLP-1, a GLP-1 analogue, insulin, an insulin analogue, or a DPP-IV inhibitor, or a pharmaceutically acceptable salt thereof.

39. The method of claim 29, wherein the subject has an HbA1c level that is equal to or greater than 6.5% of whole blood.

40. The method of claim 29, wherein the subject has an HbA1c level that is equal to or greater than 7.0% of whole blood.

41. The method of claim 29, wherein the subject has an HbA1c level that is equal to or greater than 7.5% of whole blood.

42. The method of claim 29, wherein the subject has a fasting plasma glucose level that is equal to or greater than 126 mg/dL.

43. The method of claim 29, wherein the subject is an adult human.

44. The method of claim 29, wherein the subject is a pediatric human.

45.-269. (canceled)
